SEQUENCE LISTING (8 sequences)

SEQ ID NO: 1 - DNA, 20 bp, Artificial Sequence
  source: /note=“Description of Artificial Sequence Synthetic primer”
gggtactgtggagggctttg

SEQ ID NO: 2 - DNA, 24 bp, Artificial Sequence
  source: /note=“Description of Artificial Sequence Synthetic primer”
aggaggtttgaataagccatctga

SEQ ID NO: 3 - DNA, 19 bp, Artificial Sequence
  source: /note=“Description of Artificial Sequence Synthetic primer”
gtatgcctgccgtgtgaac

SEQ ID NO: 4 - DNA, 22 bp, Artificial Sequence
  source: /note=“Description of Artificial Sequence Synthetic primer”
aaagcaagcaagcagaatttgg

SEQ ID NO: 5 - DNA, 18 bp, Artificial Sequence
  source: /note=“Description of Artificial Sequence Synthetic primer”
cggcatcgtcaccaactg

SEQ ID NO: 6 - DNA, 23 bp, Artificial Sequence
  source: /note=“Description of Artificial Sequence Synthetic primer”
aacatgatctgggtcatcttctc

SEQ ID NO: 7 - DNA, 23 bp, Artificial Sequence
  source: /note=“Description of Artificial Sequence Synthetic primer”
ggtggtctcctctgacttcaaca

SEQ ID NO: 8 - DNA, 23 bp, Artificial Sequence
  source: /note=“Description of Artificial Sequence Synthetic primer”
gttgctgtagccaaattcgttgt

(19) United States
(12) Patent Application Publication
(10) Pub. No.: US 2018/0066307 A1
(43) Pub. Date: Mar. 8, 2018

(54) EXOSOMES AND USES THEREOF

(71) Applicants: THE BROAD INSTITUTE INC., Cambridge, MA (US); MASSACHUSETTS INSTITUTE OF TECHNOLOGY, Cambridge, MA (US); PRESIDENT AND FELLOWS OF HARVARD COLLEGE, Cambridge, MA (US)

(72) Inventors: Dmitry Ter-Ovanesyan, Cambridge, MA (US); Emma Joanna Katharina Kowal, Boston, MA (US); George M. Church, Cambridge, MA (US); Aviv Regev, Cambridge, MA (US)

(21) Appl. No.: 15/790,830

(22) Filed: Oct. 23, 2017

Related U.S. Application Data
(63) Continuation-in-part of application No. PCT/US2016/029003, filed on Apr. 22, 2016.
(60) Provisional application No. 62/151,189, filed on Apr. 22, 2015.
(60) Provisional application No. 62/151,166, filed on Apr. 22, 2015.
(60) Provisional application No. 62/151,142, filed on Apr. 22, 2015.

(51) Int. Cl. C12Q 1/68

(52) U.S. Cl. CPC C12Q 1/6806; C12Q 1/686; C12Q 1/6851

(57) ABSTRACT

The present invention relates to the isolation and purification of exosomes from biological samples, and to methods for extracting RNA contained therein. The present invention provides methods and uses for the purification of exosomes, as well as compositions comprising same.

### RELATED APPLICATIONS AND INCORPORATION BY REFERENCE

This application is a continuation-in-part application of international patent application Serial No. PCT/US US2016/029003 filed Apr. 22, 2016, which published as PCT Publication No. WO 2016/172598 on Oct. 27, 2017, which claims benefit of and priority to U.S. Provisional Application Nos. 62/151,142, 62/151,166 and 62/151,189 all filed Apr. 22, 2015.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under grant numbers HG006193 and HG005550 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 16, 2016, is named 48009.99.2110_SL.txt and is 3 bytes in size.

### FIELD OF THE INVENTION

The present invention relates to the isolation and purification of exosomes from biological samples, and to methods for extracting RNA contained therein. The present invention provides methods and uses for the purification of exosomes, as well as compositions comprising same.

The present invention further relates to the use of exosomes for diagnosis and prognosis purposes. Provided are methods, uses and kits of parts useful in particular for RNA profiling, as well as for diagnostic and prognostic methods in a subject.

The present invention also relates for the use of exosomes in therapeutics. Provided are methods for treatment or prophylaxis of a disorder of interest.

### BACKGROUND OF THE INVENTION

Exosomes are small extracellular vesicles that have been shown to contain RNA.

Exosomes can be isolated using ultracentrifugation steps. However, purified exosomes have proven to be difficult to isolate. In particular, the presence of cellular debris amounts to ‘contaminant’ in a preparation, jeopardizing genetic and biochemical analysis of exosomes. While exosomes are isolated using ultracentrifugation as described herein, other methods such as filtration, chemical precipitation, size exclusion chromatography, microfluidics are known in the art.

Further, RNA content of exosomes was previously reported as uncorrelated to corresponding cellular RNA content (Skog J, Würdinger T, van Rijn S, Meijer D H, Gainche L, Sena-Esteves M, Curry W T Jr, Carter B S, Krichevsky A M, Breakefield X O. Nat Cell Biol. 2008 December; 10(12):1470-6. doi: 10.1038/ncb1800. Epub 2008 Nov. 16.).

Citation or identification of any document in this application is not an admission that such document is available as prior art to the present invention.

### SUMMARY OF THE INVENTION

It would be of interest to provide methods that allow to establish a relationship between exosomal RNA content and corresponding cellular RNA content. This would have broad diagnostic and prognostic applications.

Further, exosomes could prove useful in the therapeutics field.

The present invention proves a method for the isolation of exosomes from a biological sample. In some aspects, said method comprises:

(a) providing a biological sample comprising exosomes from a cell population,
(b) preparing an exosome-enriched fraction from the biological sample of step (a),
(c) subjecting the exosome-enriched fraction of step (b) to a treatment with a proteinase.

The present invention also provides a method for the purification of exosomes from a biological sample. In some aspects, said method comprises:

(a) providing a biological sample comprising exosomes from a cell population,
(b) preparing an exosome-enriched fraction from the biological sample of step (a),
(c) subjecting the exosome-enriched fraction of step (b) to a treatment with a proteinase.

In some aspects, the proteinase of step (c) may be one or more independently selected from serine proteases, threonine proteases, cysteine proteases, aspartate proteases, glutamic acid proteases and metalloproteases. In some aspects, step (c) may comprise a treatment with a proteinase and subsequent inactivation thereof. According to some embodiments, proteinase inactivation may be performed with one or more protease inhibitor(s). In some aspects, the proteinase of step (c) may be proteinase K.

In some aspects, step (b) may comprise one or more centrifugation steps, so as to remove live cells, dead cells and larger cellular debris from the biological sample of step (a). In some aspects, step (b) may comprise one or more filtration steps. In some embodiments, the filtration step may comprise filtration with a submicron filter, for example the submicron filter may be a 0.22 micron filter. In some aspects, wherein step (b) may comprise one or more centrifugation steps, so as to remove live cells, dead cells and larger cellular debris from the biological sample of step (a), followed by a filtration step with a submicron filter. In some aspects, step (b) may comprise one or more ultracentrifugation steps. In some aspects, step (b) may comprise:

(b-1) filtrating with a submicron filter,

(b-2) performing a first ultracentrifugation step, so as to provide a first exosome-enriched fraction,

(b-3) washing the exosome-enriched fraction of step (b-2), and

(b-4) performing a second ultracentrifugation step of the washed exosome-enriched fraction of step (b-3).

In some aspects, step (c) may be performed after the final ultracentrifugation step of step (b). In some aspects, step (c) may comprise a treatment with proteinase K and subsequent inactivation thereof. In some embodiments, the inhibitor may be diisopropyl fluorophosphate (DFP) or phenyl methane sulphonyl fluoride (PMSF).

In some aspects, the method may further comprise:

(d) subjecting the proteinase K-treated fraction of step (c) to a treatment with an RNase.

In some embodiments, the RNase may be one or more independently selected from RNase A, B, C, 1, and T1. In some embodiments, the RNase may be RNAse A/T1. In some aspects, step (d) may comprise a treatment with RNase and subsequent inactivation thereof. In some aspects, inactivation of RNase may comprise a treatment with one or more RNase inhibitor(s). In some embodiments, the RNase inhibitor may be selected from protein-based RNase inhibitors.

In some aspects, the method may provide exosomes which are essentially free of extra-exosomal material. In some aspects, the method may provide exosomes which are essentially free of extra-exosomal nucleic acid-protein complexes. In some aspects, the method may provide exosomes which are essentially free of extra-exosomal RNA-protein complexes.

In still further aspects, the method may further comprise after step (c) or (d) one or more purification steps based on the affinity of a bait molecule for a prey exosome biomarker. In an embodiment, the method comprises a cell population comprising one or more cell types, 2 or more cell types, preferably 3 or more cell types, 4 or more cell types or 5 or more cell types. In an embodiment, the method comprises isolating or purifying cell type-specific exosomes, or cell-subtype-specific exosomes. In an embodiment, the method wherein the one or more cell type comprises cells derived from the endoderm, cells derived from the mesoderm, or cells derived from the ectoderm.

In another aspect, the method comprises cells, wherein the cells derived from the endoderm comprise cells of the respiratory system, the intestine, the liver, the gallbladder, the pancreas, the islets of Langerhans, the thyroid or the hindgut. In an embodiment, the method comprises cells, wherein the cells derived from the mesoderm comprise osteochondroprogenitor cells, muscle cells, cells from the digestive systems, renal stem cells, cells from the reproductive system, bloods cells or cells from the circulatory system (such as endothelial cells). In another embodiment, the method comprises cells, wherein the cells derived from the ectoderm comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland. In an embodiment, the method comprises cells, wherein the cells from the central nervous system and the peripheral nervous system comprise neurons, Schwann cells, satellite glial cells, oligodendrocytes or astrocytes. In a further embodiment, the method comprises neurons wherein the neurons comprise interneurons, pyramidal neurons, gabaergic neurons, dopaminergic neurons, serotoninergic neurons, glutamatergic neurons, motor neurons from the spinal cord, or inhibitory spinal neurons.

In an aspect of the invention, the method provides cell types wherein the one or more cell-type is a cancer cell or a circulating tumor cell (CTC), such as cancer cell or CTC derived from any cell-types or cell subtypes. In an embodiment, the method provides a prey exosome biomarker, wherein the biomarker comprises a surface biomarker. In a further embodiment, the method wherein the prey exosome biomarker comprises a membrane protein. In another embodiment, the method comprises a prey exosome biomarker selected from the group consisting of proteins as per Table D, column G; or proteins as per Table D, column H; or proteins as per Table D, column I; or proteins as per Table D, column J; or proteins as per Table D, column K; or proteins as per Table D, column L; or proteins as per Table D, column M. In one embodiment, the prey exosome biomarker is FLRT3 and/or L1CAM.

In an aspect, the method provides a bait molecule comprising a protein and more preferentially an antibody, such as a monoclonal antibody. In an embodiment, the bait molecule is recognized by an affinity ligand. In an embodiment, the bait molecule can also be an RNA aptamer. In a further embodiment, the affinity ligand comprises a protein, a peptide, a divalent metal-based complex or an antibody. In an embodiment, the bait molecule or the affinity ligand is immobilized on a solid substrate. In another embodiment, the solid substrate is selected from a purification column, a microfluidic channel or beads, such as magnetic beads. In an embodiment, the method provides a purification, wherein the purification comprises a microfluidic affinity based purification, a magnetic based purification, a pull-down purification or a fluorescence activated sorting-based purification. In another embodiment, the method provides a biological sample, wherein the biological sample comprises a body fluid or is derived from a body fluid, wherein the body fluid was obtained from a mammal. In a further aspect, the body fluid is selected from the group consisting of amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

The present invention provides a method for the isolation of exosomes from a cell population, comprising steps of: (1) providing isolated exosomes from a biological sample comprising exosomes from said cell population, (2) performing on the isolated exosomes of step (1) one or more purification steps based on the affinity of a bait molecule for a prey exosome biomarker.

In another aspect, the present invention provides a method for the purification of exosomes from a cell population, comprising steps of: (1) providing purified exosomes from a biological sample comprising exosomes from said cell population, (2) performing on the purified exosomes of step (1) one or more purification steps based on the affinity of a bait molecule for a prey exosome biomarker.

In an embodiment, the invention provides a method for either isolation or purification of exosomes from a cell population, wherein the cell population comprises one or more cell types, 2 or more cell types, 3 or more cell types, 4 or more cell types or 5 or more cell types. In an embodiment, the method isolates or purifies cell type-specific exosomes, or cell-subtype-specific exosomes. the method comprises a cell population comprising one or more cell types, 2 or more cell types, preferably 3 or more cell types, 4 or more cell types or 5 or more cell types. In an embodiment, the method comprises isolating or purifying cell type-specific exosomes, or cell-subtype-specific exosomes. In an embodiment, the method provides for isolation or purification of exosomes from a cell population, wherein the one or more cell type comprises cells derived from the endoderm, cells derived from the mesoderm, or cells derived from the ectoderm. In another aspect, the method comprises cells, wherein the cells derived from the endoderm comprise cells of the respiratory system, the intestine, the liver, the gallbladder, the pancreas, the islets of Langerhans, the thyroid or the hindgut. In an embodiment, the method comprises cells, wherein the cells derived from the mesoderm comprise osteochondroprogenitor cells, muscle cells, cells from the digestive systems, renal stem cells, cells from the reproductive system, bloods cells or cells from the circulatory system (such as endothelial cells). In another embodiment, the method comprises cells, wherein the cells derived from the ectoderm comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland. In an embodiment, the method comprises cells, wherein the cells from the central nervous system and the peripheral nervous system comprise neurons, Schwann cells, satellite glial cells, oligodendrocytes or astrocytes. In a further embodiment, the method comprises neurons wherein the neurons comprise interneurons, pyramidal neurons, gabaergic neurons, dopaminergic neurons, serotoninergic neurons, glutamatergic neurons, motor neurons from the spinal cord, or inhibitory spinal neurons.

In an aspect of the invention, the method provides for isolation or purification of exosomes from a cell population, wherein the one or more cell-type is a cancer cell or a circulating tumor cell (CTC), such as cancer cell or CTC derived from any cell-types or cell subtypes. In an embodiment, the method provides a prey exosome biomarker, wherein the biomarker comprises a surface biomarker. In a further embodiment, the method wherein the prey exosome biomarker comprises a membrane protein. In another embodiment, the method comprises a prey biomarker selected from the group consisting of proteins as per Table D, column G; or proteins as per Table D, column H; or proteins as per Table D, column I; or proteins as per Table D, column J; or proteins as per Table D, column K; or proteins as per Table D, column L; or proteins as per Table D, column M. In one embodiment, the prey exosome biomarker is FLRT3 and/or L1CAM.

In an aspect, the method provides for isolation or purification of exosomes from a cell population, wherein the bait molecule comprises a protein and more preferentially an antibody, such as a monoclonal antibody. In an embodiment, the bait molecule is recognized by an affinity ligand. In an embodiment, the bait molecule can also be an RNA aptamer. In a further embodiment, the affinity ligand comprises a protein, a peptide, a divalent metal-based complex or an antibody. In an embodiment, the bait molecule or the affinity ligand is immobilized on a solid substrate. In another embodiment, the solid substrate is selected from a purification column, a microfluidic channel or beads, such as magnetic beads. In an embodiment, the method provides a purification, wherein the purification comprises a microfluidic affinity based purification, a magnetic based purification, a pull-down purification or a fluorescence activated sorting-based purification.

The present invention also provides a method for the preparation of exosomal RNA from a biological sample, said method comprising:

(i) providing a biological sample comprising exosomes from a cell population,
(ii) preparing purified exosomes from the biological sample of step (i),
(iii) extracting RNA from the purified exosomes of step (i).

In some aspects, step (ii) may comprise the method for the isolation/purification of exosomes as disclosed herein. In other aspects, the method for the preparation of exosomal RNA from a biological sample comprises a method wherein the purified exosomes prepared at step (ii) are exosomes from a single cell type or from a single cell subtype.

The present invention provides a method for the preparation of exosomal RNA of a cell population, comprising steps of: (1) providing purified exosomes from a biological sample comprising exosomes from said cell population; (2) performing on the purified exosomes of step (1) one or more purification steps based on the affinity of a bait molecule for a prey exosome biomarker, and (3) extracting RNA from the purified exosomes of step (2).

In some aspects, the exosomal RNA may be total exosomal RNA. In some aspects, the exosomal RNA may comprise exosomal messenger RNA. In some aspects, the exosomal RNA may be total exosomal messenger RNA. In some aspects, the exosomal RNA is exosomal RNA from single cell type exosomes or single cell subtype exosomes.

The present invention also provides for a use of a proteinase in the purification of exosomes from a biological sample. The present invention also provides for a use of a proteinase and of an RNase in the purification of exosomes from a biological sample. The present invention also provides for a use of a proteinase in the purification of an ultracentrifugated exosome-containing sample. The present invention also provides for a use of a proteinase and of an RNase in the purification of an ultracentrifugated exosome-containing sample.

In the uses of the invention, in some aspects, the proteinase may be proteinase K. In the uses of the invention, in some aspects, the ultracentrifugated exosome-containing sample may be a washed ultracentrifugated exosome-containing sample.

In the uses of the invention, in some aspects, the ultracentrifugated exosome-containing sample may be a washed ultracentrifugated exosome-containing sample.

In the methods and uses of the invention as disclosed herein, in some aspects, the biological sample may be a bodily fluid or is derived from a bodily fluid, wherein the bodily fluid was obtained from a mammal. In some embodiments, the bodily fluid may be selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In the methods and uses of the invention as disclosed herein, in some aspects, the cell population may be a population of cells of the same cell type. In the methods and uses of the invention as disclosed herein, in some aspects, the cell population is a population of cells of different cell types. In another embodiment, the cell population comprises one or more cell types, 2 or more cell types, 3 or more cell types, 4 or more cell types, or 5 or more cell types.

In the methods and uses of the invention as disclosed herein, in some aspects, the biological sample comprises cultured cells. In some embodiments, the biological sample may comprise cells cultured in vitro. In some embodiments, the biological sample may comprise cells cultured ex vivo. In some embodiments, the biological sample may be a sample obtained by liquid biopsy. In some embodiments, the biological sample may comprise a cell type selected from cells types present in amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit.

The present invention also provides exosome preparations and compositions comprising exosomes. The present invention provides an exosome preparation obtainable with the method or the use as disclosed herein. The present invention also provides a composition comprising exosomes, wherein the composition is essentially free of extra-exosomal material. The present invention also provides a composition comprising exosomes, wherein the composition is essentially free of extra-exosomal nucleic acid-protein complexes. The present invention also provides a composition comprising exosomes, wherein the composition is essentially free of extra-exosomal RNA-protein complexes. In another aspect, the invention provides a composition comprising cell type specific exosomes or cell subtype specific exosomes. In an embodiment, the composition comprises exosomes, wherein the exosomes are specific for one or more cell types or cell subtypes. In another embodiment, the composition comprises purified exosomes, wherein said purified exosomes are exosomes from a single cell-type or of a single cell subtype.

The present invention provides a method for the determination of cellular RNA content in a cell population. In some aspects, said method comprises:

(a) providing a biological sample comprising exosomes from said cell population,
(b) preparing purified exosomes from the sample of step (a),
(c) extracting RNA from the purified exosomes of step (b), so as to provide exosomal RNA,
(d) analyzing the exosomal RNA extracted at step (c),
(e) estimating, as a function of the result from step (d), the cellular RNA content in the cell population.

In some aspects, step (b) may comprise the method for the purification of exosomes as disclosed herein.

In some aspects, the invention provides a method for the determination of cellular RNA content of a cell population, said method comprising (a) providing a biological sample comprising exosomes from said cell population; (b) preparing purified exosomes from the sample of step (a); (c) extracting RNA from the purified exosomes of step (b), so as to provide exosomal RNA; (d) analyzing the exosomal RNA extracted at step (c); (e) estimating, as a function of the result from step (d), the cellular RNA content in the cell population; wherein step (b) further comprises performing on the purified exosomes one or more purification steps based on the affinity of a bait molecule for a prey exosome biomarker.

In an embodiment, the method comprises the method of step (b) wherein the method comprises the isolation or the purification of exosomes from a biological sample. In an embodiment, the invention provides a method for either isolation or purification of exosomes from a cell population, wherein the cell population comprises one or more cell types, 2 or more cell types, 3 or more cell types, 4 or more cell types or 5 or more cell types. In an embodiment, the method isolates or purifies cell type-specific exosomes, or cell-subtype-specific exosomes. the method comprises a cell population comprising one or more cell types, 2 or more cell types, preferably 3 or more cell types, 4 or more cell types or 5 or more cell types. In an embodiment, the method comprises isolating or purifying cell type-specific exosomes, or cell-subtype-specific exosomes. In an embodiment, the method provides for isolation or purification of exosomes from a cell population, wherein the one or more cell type comprises cells derived from the endoderm, cells derived from the mesoderm, or cells derived from the ectoderm. In another aspect, the method comprises cells, wherein the cells derived from the endoderm comprise cells of the respiratory system, the intestine, the liver, the gallbladder, the pancreas, the islets of Langerhans, the thyroid or the hindgut. In an embodiment, the method comprises cells, wherein the cells derived from the mesoderm comprise osteochondroprogenitor cells, muscle cells, cells from the digestive systems, renal stem cells, cells from the reproductive system, bloods cells or cells from the circulatory system (such as endothelial cells). In another embodiment, the method comprises cells, wherein the cells derived from the ectoderm comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland. In an embodiment, the method comprises cells, wherein the cells from the central nervous system and the peripheral nervous system comprise neurons, Schwann cells, satellite glial cells, oligodendrocytes or astrocytes. In a further embodiment, the method comprises neurons wherein the neurons comprise interneurons, pyramidal neurons, gabaergic neurons, dopaminergic neurons, serotoninergic neurons, glutamatergic neurons, motor neurons from the spinal cord, or inhibitory spinal neurons.

In an aspect of the invention, the method provides for isolation or purification of exosomes from a cell population, wherein the one or more cell-type is a cancer cell or a circulating tumor cell (CTC), such as cancer cell or CTC derived from any cell-types or cell subtypes. In an embodiment, the method provides a prey exosome biomarker, wherein the biomarker comprises a surface biomarker. In a further embodiment, the method wherein the prey exosome biomarker comprises a membrane protein. In another embodiment, the method comprises a prey exosome biomarker selected from the group consisting of proteins as per Table D, column G; or proteins as per Table D, column H; or proteins as per Table D, column I; or proteins as per Table D, column J; or proteins as per Table D, column K; or proteins as per Table D, column L; or proteins as per Table D, column M. In one embodiment, the prey exosome biomarker is FLRT3 and/or L1CAM.

In an aspect, the method provides for isolation or purification of exosomes from a cell population, wherein the bait molecule comprises a protein and more preferentially an antibody, such as a monoclonal antibody. In an embodiment, the bait molecule is recognized by an affinity ligand. In an embodiment, the bait molecule can also be an RNA aptamer. In a further embodiment, the affinity ligand comprises a protein, a peptide, a divalent metal-based complex or an antibody. In an embodiment, the bait molecule or the affinity ligand is immobilized on a solid substrate. In another embodiment, the solid substrate is selected from a purification column, a microfluidic channel or beads, such as magnetic beads. In an embodiment, the method provides a purification, wherein the purification comprises a microfluidic affinity based purification, a magnetic based purification, a pull-down purification or a fluorescence activated sorting-based purification.

In some aspects, step (e) may be performed based on a predicted correlation between exosomal RNA content and cellular RNA content.

In some aspects, said determination may comprise a qualitative determination. In some aspects, said determination may comprise a quantitative determination. In some embodiments, said quantitative determination may comprise determination of relative abundance of two RNAs. In some aspects, said determination may comprise determination of mRNA profiles.

In some aspects, said RNA may comprise messenger RNA (mRNA). In some aspects, said RNA may comprise micro RNA (miRNA). In some aspects, said RNA may comprise long non-coding RNA (IncRNA).

In some aspects, step (D) may comprise a qualitative determination. In some aspects, step (D) may comprise a quantitative determination. In some aspects, step (D) may comprise RNA sequencing (RNA seq). In some aspects, step (D) may comprise array analysis. In some aspects, step (D) may comprise reverse transcription polymerase chain reaction (RT-PCR). In some aspects, step (D) may comprise quantitative reverse transcription polymerase chain reaction (qRT-PCR). In some aspects, step (D) may comprise analyzing one or more sequence/s of interest.

In some aspects, the method of the invention comprises testing for the presence or absence of said sequence/s of interest. In some embodiments, step (D) may comprise analyzing for one or more allelic variants of a sequence of interest.

In some aspects, step (D) may comprise testing for presence or absence of said allelic variants. In some aspects, step (D) may comprise genome-wide analysis. In some aspects, step (D) may comprise transcriptome profiling.

In some aspects, the determination may be time-lapse.

In some aspects, the cell population may be a population of cells of the same cell type. In some aspects, the cell population may be a population of cells of different cell types.

In some aspects, the biological sample may comprise cultured cells. In some aspects, the biological sample may comprise cells cultured in vitro. In some aspects, the biological sample may comprise cells cultured ex vivo. In some aspects, the biological sample may be a sample obtained by liquid biopsy. In some aspects, the biological sample may comprise a cell type selected from blood, epithelia, muscle and neural cell types.

In some aspects, the biological samples is obtained from a body fluid, selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

In some aspects, the cell population of step (a) may be isolated as a subpart of a larger initial cell population. In some aspects, the cell population of step (a) may be obtained from a body fluid and isolated by immuno-magnetic separation.

In some aspects, the method of the invention may be for use in diagnosis. In some aspects, the method of the invention may be for use in prognosis. In some aspects, the method of the invention may be for use in identifying markers. In some aspects, the method of the invention may be for use in a screening process. In another aspect, the method determines the cellular RNA content of a single cell type or of a single cell subtype.

The present invention also provides a method for the diagnostic or prognostic of a disorder of interest in a subject. In some aspects, the method may comprise:

(I) selecting a marker, wherein said marker is associated with said disorder and wherein said marker may be determined in a cell type that is found in the subject to be in contact with a body fluid,
(II) providing a biological sample from said body fluid from said subject,
(III) estimating the cellular RNA content of said marker in the biological sample of step (II) by performing the method for the determination of cellular RNA content in a cell population as disclosed herein.

In an embodiment, the invention provides a method for the diagnostic or prognostic of a disorder of interest in a subject, wherein the cellular RNA content is the cellular content of a single cell type or of a single cell subtype.

In some aspects, the method further comprises:

(IV) determining, from the results of step (III), the status of the marker selected at step (I).

In some aspects, the marker may be selected from expression of a given open reading frame (ORF), overexpression of a given open reading frame (ORF), repression of a given open reading frame (ORF), over-repression of a given open reading frame (ORF), expression of a given allelic variant, relative level of expression of a given open reading frame (ORF), presence of a mutation in a given open reading frame (ORF).

In some aspects, said disorder may be a blood disorder and said marker is a marker that may be determined in one or more cell type/s that is/are found in the subject to be in contact with blood.

In some aspects, said disorder may be a brain or spine disorder and said marker may be a marker that may be determined in one or more cell type/s that is/are found in the subject to be in contact with cerebrospinal fluid.

In some aspects, said disorder may be a heart disorder and said marker may be a marker that may be determined in one or more cell type/s that is/are found in the subject to be in contact with blood or pericardial fluid.

In some aspects, said disorder may be said disorder is a prostate or bladder disorder and said marker may be a marker that may be determined in one or more cell type/s that is/are found in the subject to be in contact with urine.

In some aspects, said disorder may be an eye disorder and said marker may be a marker that may be determined in one or more cell type/s that is/are found in the subject to be in contact with tears.

In some aspects, said disorder may be a lung disorder and said marker may be a marker that may be determined in one or more cell type/s that is/are found in the subject to be in contact with pleural fluid.

The present invention also provides compositions comprising exosomes. In some aspects, the composition may be essentially free of extra-exosomal material, for use in diagnostics. In some aspects, the composition may be essentially free of extra-exosomal nucleic acid-protein complexes. In some aspects, the composition may be essentially free of extra-exosomal RNA-protein complexes.

The present invention provides a method for the treatment or prophylaxis of a disorder in a patient. In some aspects, said method may comprise exosome-mediated delivery of a therapeutic RNA to a cell.

In some aspects, said exosome-mediated delivery may occur from one donor cell to a recipient cell, and wherein the therapeutic RNA may result from transcription in the donor cell.

In some aspects, transcription in the donor cell may be inducible.

In some aspects, the delivery may be performed ex vivo. In some aspects, the delivery may be performed in vivo.

The present invention also an exosome for use in therapy. In some aspects, the present invention provides an exosome for use in delivering a therapeutic RNA to a cell. In some aspects, the exosome may be produced in vitro. In some aspects, the exosome may be produced in vivo.

The present invention also provides a therapeutic RNA for use in exosome-mediated delivery to a cell. In some aspects, the exosome may be produced in vitro. In some aspects, the exosome may be produced in vivo.

The present invention also provides a pharmaceutical composition comprising an exosome. In some aspects, said exosome may comprise a therapeutic RNA for delivery into a cell. In some aspects, the delivery may be performed ex vivo. In some aspects, the delivery may be performed in vivo. In some aspects, the cell may be capable of producing exosomes comprising a therapeutic RNA. In some aspects, the pharmaceutical composition is in a form suitable for injection.

The present invention also provides a use of a therapeutic RNA in the manufacture of a medicament for the treatment or prophylaxis of a disorder in a patient. In some aspects, the RNA may be delivered to a cell in an exosome-packaged form. In some aspects, the exosome may comprise a therapeutic RNA or delivery into a cell.

In the method, composition or use as disclosed herein, the therapeutic RNA may be translated in the recipient cell.

In the method, composition or use as disclosed herein, the therapeutic RNA may be a small interfering RNA (siRNA).

In the method, composition or use as disclosed herein, the therapeutic RNA may be a short hairpin RNA (shRNA).

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows graph of RNA fluorescence unit (FU) plotted against RNA size (nt) for various exosome purification methods.

FIGS. 2A-2D show electron microscopy (EM) photographs of exosome preparations for various exosome purification methods; (2A) Electron microscopy of exosomes with no treatment, (2B) Electron microscopy of exosomes with proteinase treated after spins: (2C) Side-by-side comparison of EM of untreated versus proteinase-treated; (2D) Electron microscopy of exosomes with proteinase treated between spins.

FIG. 3 shows results of a qRT-PCR experiment for various exosome purification methods.

FIG. 4A-4C show RNA-Seq data, showing that the RNA profile of mRNAs in exosomes reflects that of the donor cells; (4A) illustrates mRNA profile in exosomes: PTMS; (4B) illustrates mRNA profile in exosomes: MT2A; (4C) illustrates mRNA profile in exosomes: Rab13.

FIGS. 5A-5K show principle and results for fluorescence imaging of cells using EU click chemistry, to assess possible exosome-mediated RNA transfer between cells; (5A) shows intercellular communication (5B) shows click-chemistry with 5-ethynyl uridine (5C) shows control HEK 293 cells grown in presence of 5-ethynyl uridine, (5D) shows negative control of HEK 293 cells with no 5-ethynyl uridine; (5E) illustrates RNA transfer experiment; (5F) shows negative control of HEK 293/K562 cells with no 5-ethynyl uridine (5G) shows negative control of HEK 293/K562 cells with no 5-ethynyl uridine with 640× magnification zoomed in; (5H) shows experimental #1 of HEK 293/K562 cells with 5-ethynyl uridine (5I) shows experimental #1 of HEK 293/K562 cells with 5-ethynyl uridine (6J) shows experimental #1 of HEK 293/K562 cells with 5-ethynyl uridine (zoomed in); (5K) shows experimental #2 of HEK 293/K562 cells with 5-ethynyl uridine.

FIGS. 6A-6D show principle and results of an experiment to assess possible exosome mediated RNA transfer between co-cultured cell lines; (6A) illustrates an alternative experiment of mouse-human co-culture; (6B) shows the experimental design; (6C) percentage of mouse genes with TMM >2; (6D) shows mouse gene expression in human cells.

FIGS. 7A-7D illustrates Poly A selected from mRNA from two replicates of K562 cells and their exosomes was compared using RNA-Seq; (7A) compares cell 1 versus cell 2; (7B) compares exosome 1 versus exosome 2; (7C) compares cell 1 versus exosome 1 (7D) compares cell 2 versus exosome 2.

FIG. 8 illustrates that mRNA is inside the exosomes.

FIG. 9 illustrates Poly A enriched mRNA from untreated exosomes and proteinase/Rnase treated exosomes was compared using RNA-Seq.

FIG. 10 illustrates targeted pull down exosome subpopulations based on their protein marker using antibody conjugated magnetic beads.

FIG. 11 illustrates exosomes which were isolated from human CSF and mRNA for four genes (detected by qRT-PCR.) Cell RNA is used as a comparison.

### DETAILED DESCRIPTION OF THE INVENTION

The terms “exosomes”, “micro-vesicles” and “extracellular vesicles” are herein used interchangeably. They refer to extracellular vesicles, which are generally of between 30 and 200 nm, for example in the range of 50-100 nm in size. In some aspects, the extracellular vesicles can be in the range of 20-300 nm in size, for example 30-250 nm in size, for example 50-200 nm in size. In some aspects, the extracellular vesicles are defined by a lipidic bilayer membrane.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R.I. Freshney, ed. (1987)).

In aspects of the invention functional genomics screens allow for discovery of novel human and mammalian therapeutic applications, including the discovery of novel drugs, for, e.g., treatment of genetic diseases, cancer, fungal, protozoal, bacterial, and viral infection, ischemia, vascular disease, arthritis, immunological disorders, etc. As used herein assay systems may be used for a readout of cell state or changes in phenotype include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmittor release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs.

In the purification methods of the invention, it was found advantageous to perform a proteinase treatment, especially after the final ultracentrifugation step carried out for exosome preparation. Without being bound by theory, it is hypothesized that such treatment allows the removal of non exosomal nucleic acid-protein complexes, such as RNA-protein complexes. The proteinase treatment (and inactivation thereof), may then be followed by an RNAse treatment.

The exosome purification methods of the invention allows one to prepare compositions comprising exosomes, wherein the composition is essentially free of extra-exosomal material, and/or essentially free of extra-exosomal nucleic acid-protein complexes, and/or essentially free of extra-exosomal RNA-protein complexes. Such compositions may be used for exosomal RNA preparation.

The purification method of the invention may include the following: removal of live cells, dead cells and larger cell debris, which may be performed by centrifugation steps and collection of the corresponding supernatants; filtration using a submicron filter such as a 0.22 micron filter; collection of exosomes by ultracentrifugation (typically at 100 g-130,000 g, for example 120,000 g); washing exosomes before an additional ultracentrifugation step; proteinase treatment and inactivation; RNase treatment and inactivation.

According to one aspect of the invention, a strong correlation can advantageously be established between the RNA profile, and notably the mRNA profile, of isolated or purified exosomes and the RNA profile of the corresponding donor cells. In particular, a correlation has been shown between the mRNA profile of exosomes from K562 cells which have been isolated or purified as per the purification method or the invention, notably after treatment with protease and then RNAse, and the RNA profile of donor K562 cells. Such a correlation has been shown for the first time and is advantageous for diagnostic applications, as the transcriptome profile from exosomes of a cell population very faithfully reflects the corresponding cellular transcriptome.

Furthermore, a correlation can also be established between the RNA content (notably the mRNA content) of purified or isolated exosomes treated with protease and RNase and the RNA content of protease/RNAse untreated exosomes. These results illustrate that the analyzed RNA content of exosomes isolated or purified as per the purification method of the invention is actually inside said exosomes and not simply externally associated with exosomes. Analyses of the RNA exosomal content can be performed using any transcriptomics method (see notably Wang et. al, Nature Review Genetics (10) 57-63), such as RNA seq (for which a princeps protocol is notably described in Macosko E Z et al., 2015, Cell 161, 1202-1214), RT-PCT (notably qRP-PCR), small RNA sequencing (Li et. al, NAR 41(6) 3619-3634) or microarray. RNA analysis can also be performed as described in “Chip-based analysis of exosomal mRNA mediating drug resistance in glioblastoma”. Shao H, Chung J, Lee K, Balaj L, Min C, Carter B S, Hochberg F H, Breakefield X O, Lee H, Weissleder R. Nat Commun. 2015 May 11; 6:6999. doi: 10.1038/ncomms7999. PMID: 25959588; “Microfluidic isolation and transcriptome analysis of serum microvesicles”. Chen C, Skog J, Hsu C H, Lessard R T, Balaj L, Wurdinger T, Carter B S, Breakefield X O, Toner M, Irimia D. Lab Chip. 2010 Feb. 21; 10(4):505-11. doi: 10.1039/b916199f. Epub 2009 Dec. 8. PMID: 20126692.

In some aspects, the purification method of the invention may further comprise a step of separating one or more sub-populations of exosomes from a purified pool of exosomes. Indeed in some aspects of the invention, a sub-population of exosomes from a mixed exosome population, found for example in a biological sample obtained from a body fluid, can be further purified or isolated, for example according to one or more specific donor cell types or donor cell subtypes. In some aspects, the purification method of the invention allows to isolate or purify subpopulations of exosomes from one or more cell types or cell subtypes, preferentially from a single cell type, or from a single cell subtype.

In some aspects, a cell population can comprise one or more cell types, notably 2 or more cell types, 3 or more cell types, 4 or more cell types, or 5 or more cell types. In some aspects, a cell population comprises at least 1 to 40 cell types, notably at least 1 to 30, at least 5 to 20, at least 5 to 10, at least 2 to 8 or at least 2 to 5 cell types. Therefore, cell type or cell subtype exosomes can be purified from a mixed exosome population obtained from a cell population.

In some aspects, cell types according to the invention comprises cell types derived from the endoderm, cell types derived from the mesoderm, or cell types derived from the ectoderm. Cell types derived from the endoderm can comprise cell types of the respiratory system, the intestine, the liver, the gallbladder, the pancreas, the islets of Langerhans, the thyroid or the hindgut. Cell types derived from the mesoderm can comprise osteochondroprogenitor cells, muscle cells, cell types from the digestive system, renal stem cells, cell types from the reproductive system, bloods cell types or cell types from the circulatory system (such as endothelial cells). Cell types derived from the ectoderm can comprise epithelial cells, cell types of the anterior pituitary, cell types of the peripheral nervous system, cell types of the neuroendocrine system, cell types of the teeth, cell types of the eyes, cell types of the central nervous system, cell types of the ependymal or cell types of the pineal gland. For example, a cell population from the central and peripheral nervous system can comprise cell types such as neurons, Schwann cells, satellite glial cells, oligodendrocytes or astrocytes. In some aspects of the invention, the one or more cell types comprise cancer cells or circulating tumor cells. Preferentially, said cancer cells or CTCs derive from the cell types as listed above. A cell type can also encompass one or more cell subtypes, notably 2 or more, 3 or more, 4 or more, 5 or more and up to 10 or more cell subtypes. For example neurons encompass various cell subtypes such as for example interneurons, pyramidal neurons, gabaergic neurons, dopaminergic neurons, serotoninergic neurons, glutamatergic neurons, motor neurons from the spinal cord, or inhibitory spinal neurons. Different cell types or cell subtypes can also be discriminated according to their respective transcriptome profile.

In some aspects, purification or isolation or exosomes according to a specific cell type or a cell subtype is achieved through one or more purification steps. In some aspects the one or more purification steps are based on the affinity of a bait molecule for a prey exosome biomarker.

In some aspects, bait molecules may be an antibody that binds exosome transmembrane protein. In some aspects, a bait molecule may be an RNA aptamer.

Prey exosome biomarkers according to the invention can be specific for one or more cell types or cell subtypes. Preferentially, prey exosomes biomarkers are membrane proteins. In this context, analysis of exosomal RNA content is highly relevant for diagnostic applications, as compared to the analysis of circulating DNA or RNA because it allows identification of the donor cell type or cell subtype though specific trans-membrane protein affinity purification, such as protein pull-up.

Exosome biomarkers can be typically identified through mass spectrometry analyses of exosomes obtained from specific cell types or cell subtypes, and if required confirmed through western blotting or qRT-PCR analysis in said exosomes. For example exosomes from induced pluripotent stem cells (IPS cells) or IPS-derived-neurons can be used, but exosomes from any cell types or cell subtypes as defined above can be subjected to mass spectrometry analysis for identification of specific trans-membrane protein biomarkers. For example, mass spectrometry analysis can also be performed on total exosomes from a body fluid, such as CSF. Analysis of the transcriptome of CSF exosomes is of high interest because such exosome population is specific of the brain cell population.

Data obtained from such mass spectrometry analysis can be combined with genome or transcriptome analysis of corresponding donor cells in order to identify relevant biomarkers. This facilitates the identification of relevant exosome biomarkers useful for the present invention. For example, regarding CNS genetic information, lists of genes are available from e.g. “Establishing the Proteome of Normal Human Cerebrospinal Fluid” Schutzer S E et al., PLoS One, 2010; 5(6): e10980. “An RNA-Sequencing Transcriptome and Splicing Database of Glia, Neurons, and Vascular Cells of the Cerebral Cortex” Zhang Y et al., The Journal of Neuroscience, 2014, 34(36):11929-11947. “Purification and Characterization of Progenitor and Mature Human Astrocytes Reveals Transcriptional and Functional Differences with Mouse” Zhang et al., 2016, Neuron 89, 37-53.

In some aspects of the invention, prey exosome biomarkers from neurons comprise one or more selected from proteins as per Table D, column G; or proteins as per Table D, column H; or proteins as per Table D, column I; or proteins as per Table D, column J; or proteins as per Table D, column K; or proteins as per Table D, column L; or proteins as per Table D, column M. In one embodiment, the prey exosome biomarker is FLRT3 and/or L1CAM. The presence of the at least one of these trans-membrane protein biomarkers in neuron exosomes can be confirmed through western blotting or RT-PCT analysis or neuron exosomes.

“Dysfunctionally phosphorylated type 1 insulin receptor substrate in neural-derived blood exosomes of preclinical Alzheimer's disease”. Kapogiannis D, Boxer A, Schwartz J B, Abner E L, Biragyn A, Masharani U, Frassetto L, Petersen R C, Miller B L, Goetzl E J. FASEB J. 2015 February; 29(2):589-96. doi: 10.1096/fj.14-262048. Epub 2014 Oct. 23. PMID: 25342129 and “Plasma exosomal α-synuclein is likely CNS-derived and increased in Parkinson's disease”. Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J. Acta Neuropathol. 2014 November; 128(5):639-50. doi: 10.1007/s00401-014-1314-y. Epub 2014 Jul. 6. PMID: 24997849 describe analysis of exosomes obtained from plasma, but as such do not provide informative or conclusive evidence establishing a relationship with a specific organ of origin (such as brain) or specific tissue of origin or a fortiori specific cell types of origin such as neurons. This is because of the circulating nature of plasma that comes into contact with a number of various organs, tissues, etc., and thus may comprise exosomes stemming from a plurality of different cell types altogether. Further, it is unclear whether some exosomes are capable of corrsing the blood brain barrier. As a consequence, the data reported in these papers do not allow to identify the exact origin of the exosomes, and in particular cannot relate to exosomes from a specific cell type (such as neurons). Further, these papers do not disclose any RNA profiling, in particular, no RNA-seq analysis.

By contrast, the present invention provides methods for accessing information on tissue- or cell-type-specific exosomes, in particular tissue- or cell-type-specific transcription profiles. The present invention also provides very-high resolution diagnostic methods, wherein a subtle change in transcription profiles (e.g. a small up- or down-regulation in the transcription of a given gene in a given cell type or a given cell sub-type) can advantageously be efficiently detected, while it could not be in a total RNA or total exosome analysis.

In some aspects the one or more purification steps can comprise a microfluidic affinity based purification (see for example “Chip-based analysis of exosomal mRNA mediating drug resistance in glioblastoma”. Shao H, Chung J. Lee K, Balaj L, Min C, Carter B S, Hochberg F H, Breakefield X O, Lee H, Weissleder R. Nat Commun. 2015 May 11; 6:6999. doi: 10.1038/ncomms7999. PMID: 25959588; “Microfluidic isolation and transcriptome analysis of serum microvesicles”. Chen C, Skog J, Hsu C H, Lessard R T, Balaj L, Wurdinger T, Carter B S, Breakefield X O, Toner M, Irimia D. Lab Chip. 2010 Feb. 21; 10(4):505-11. doi: 10.1039/b916199f. Epub 2009 Dec. 8. PMID: 20126692.), a magnetic based purification, a pull-down purification or a fluorescence activated vesicle sorting-based purification (FAVS, see for example Van der Pol E et al., J Thromb Haemost., 2013 June; 11 Suppl 1:36-45 “Innovation in detection of microparticles and exosomes” and Van des Pol E. et al., J Thromb Haemost. 2012 May; 10(5):919-30), “Single vs. swarm detection of microparticles and exosomes by flow cytometry”; “Glypican-1 identifies cancer exosomes and detects early pancreatic cancer”. Melo S A, Luecke L B, Kahlert C, Fernandez A F, Gammon S T, Kaye J, LeBleu V S, Mittendorf E A, Weitz J, Rahbari N, Reissfelder C, Pilarsky C, Fraga M F, Piwnica-Worms D, Kalluri R. Nature. 2015 Jul. 9; 523(7559):177-82. doi: 10.1038/nature14581. Epub 2015 Jun. 24. PMID: 26106858). Commercial precipitation kits like ExoQuick™ and Total Exosome Isolation™ precipitation solutions are also available. Such kits are easy to use with only 1 or 2 steps and do not require any expensive equipment or advanced technical know-how.

In some aspects, the bait molecule can be a bait protein, such as an antibody and in some aspects is preferentially a monoclonal antibody directed against a prey exosome biomarker. In some aspects, the bait molecule can also be an RNA aptamer. If several prey exosomes are to be combined for purification, a mix of corresponding monoclonal antibodies directed against each of the said prey exosomes biomarkers to be pull-up can be used.

In some aspects, the bait molecule is recognized by an affinity ligand. Said affinity ligand can be a divalent metal-based complex, a protein, a peptide such as fusion protein tag or more preferentially an antibody.

In some aspects, the bait molecule or the affinity ligand is immobilized or “coupled” directly, or indirectly to a solid substrate material such as by formation of covalent chemical bonds between particular functional groups on the ligand (for example primary amines, thiols, carboxylic acids, aldehydes) and reactive groups on the substrate. A substrate, or a matrix, in the affinity purification steps of the method of the invention can be any material to which a biospecific ligand (i.e., the bait molecule or the affinity ligand) is coupled. Useful affinity supports may be those with a high surface-area to volume ratio, chemical groups that are easily modified for covalent attachment of ligands, minimal nonspecific binding properties, good flow characteristics and/or mechanical and chemical stability. Several substrates may be utilized as solid substrate, including for example agarose, cellulose, dextran, polyacrylamide, latex or controlled pore glass. Magnetic particles may also be used as a substrate instead of beaded agarose or other porous resins. Their small size provides the sufficient surface area-to-volume ratio needed for effective ligand immobilization and affinity purification. Magnetic beads may be produced as superparamagnetic iron oxide particles that may be covalently coated with silane derivatives. The coating makes the beads inert (i.e., to minimize nonspecific binding) and provides the particular chemical groups needed for attaching any affinity ligands of interest. Affinity purification with magnetic particles is generally not performed in-column. Instead, a few microliters of beads may be mixed with several hundred microliters of sample as a loose slurry. During mixing, the beads remain suspended in the sample solution, allowing affinity interactions to occur with the immobilized ligand. After sufficient time for binding has been given, the beads are collected and separated from the sample using a powerful magnet. An exemplary bead purification method can be found in “Proteomic comparison defines novel markers to characterize heterogeneous populations of extracellular vesicle subtypes”. Kowal J, Arras G, Colombo M, Jouve M, Morath J P, Primdal-Bengtson B, Dingli F, Loew D, Tkach M, Théry C. Proc Natl Acad Sci USA. 2016 Feb. 23; 113(8):E968-77. doi: 10.1073/pnas. 1521230113. Epub 2016 Feb. 8. PMID: 26858453.

In some aspects of the invention, a pull down assay can be performed for the purification or isolation of a subpopulation of exosomes by pulling-down of one or more specific prey exosome biomarkers (preferentially a membrane protein as described below). Said prey exosome biomarkers may be specific of a at least one cell type or cell subtype and advantageously lead to enriching in exosomes from said selected cell type or cell subtype.

In some aspects the at least one or more purification steps for the purification of an exosome subpopulation comprise a pull down purification. In such pull-down purification, the prey exosome biomarker is generally a (trans)membrane protein, which has been found to be expressed in a cell type or a cell subtype. The bait protein is preferentially a monoclonal antibody directed against any of the prey exosome biomarker(s) which is to be pulled-up. Magnetic beads (for example Dynabeads® from Thermo Fisher Scientific) coated with an affinity ligand for the bait protein can be used to isolate said bait protein bound to said prey exosome biomarker(s). The affinity ligand is preferentially a class specific or a species specific antibody. As a matter of example, magnetic beads coated with anti-mouse antibodies can be used together with monoclonal mouse antibodies directed against a specific surface protein of a cell type or cell subtype subpopulation of exosomes (such as for example CD63 or CD81). Generally, a control antibody, such as a mouse mcherry monoclonal antibody, can be used.

A pull down assay can therefore be used to illustrate and validate the purification, or isolation of at least two exosome subpopulations expressing each at least one specific membrane protein, such as the canonical exosomes markers CD63 and CD81, which have previously been pooled. As shown in the results examples, said at least two exosomes subpopulations can be re-separated based on the selected protein biomarker. The purification or isolation of exosome subpopulations by at least one specific prey exosome biomarker (preferentially a membrane protein) can be further confirmed using western blot or qRT-PCT.

Several control experiments can also be envisioned to compare the transcriptome of subpopulation of exosomes, purified or isolated by pull-up of at least one specific exosome biomarker, according to the method of the invention.
It is advantageously possible to compare the transcriptome profile of at least two subpopulations of exosomes, purified from a mixed exosome population (e.g.: obtained from a cell population comprising one or more cell types, such as the K562 cells) using specific exosome biomarkers (such as CD63 or CD81) as described above (e.g.: using magnetic beads pull-down purification). The transcriptome profile of said exosomes subpopulations can also be further compared to the transcriptome profile of the total exosome population. Typically RNA seq analysis of exosomes is particularly well suited for such transcriptome comparisons.
It is advantageously possible to compare the RNA seq analysis of total RNA, mRNA, micro RNA (miRNA), or long non coding RNA (IncRNA) of (i) at least one cell type and (ii) exosomes obtained from said at least one cell type. As a matter of example, it is possible to perform RNA seq analysis of mRNA from (i) IPS cells and IPS-derived neurons, and (ii) exosomes obtained respectively from said IPS cells and IPS-derived neurons and then compare the obtained results.
It is advantageously possible to compare (i) transcriptome profile analysis (notably the RNA seq analysis) of exosomes from the said different cell types or subtypes, isolated according through the purification method of the invention (notably using antibody-conjugated magnetic beads as described above) in order to enrich for exosomes expressing at least one cell type or cell subtype specific biomarker, with (ii) the transcriptome profile of total exosomes. For example the RNA seq results of exosomes from IPS cells and neuron exosomes isolated according to the pull down assay as described above can be compared to the RNA profile of total exosomes from both cell types.
In vitro experiments for the control of the purification of exosome subpopulations can also comprise experiments, wherein exosomes subpopulations are purified or isolated from a complex biological sample obtained from at least two cell populations, cell types, or cell subtypes. For example, from a mix of media obtained from cell culture of different cell types such as IPS cells and neurons. Exosomes of the specific cells types are then purified as described above and their transcriptome is analysed. Such an experiment allows reconstructing, ex post facto, the transcriptome of the original cell type.

Isolation or purification of total exosomes from biological samples derived from any body fluid such as CSF, urine, or blood etc. and transcriptome analysis of the obtained exosome population can also be envisioned. Using cell-type specific biomarkers, exosome subpopulations can be further purified through any of the purification steps as described above, and enrichment in expression of specific cell type biomarkers can be searched through transcriptome analysis of this subpopulation as compared to the total exosome population. Said analysis is of particular interest for CSF analysis and identification of exosomes from specific neuronal subtypes

According to the present invention, the RNA content of exosomes is found to correlate the RNA content of the corresponding cell. In other terms, in particular when exosomes are purified in accordance with purification method of the present invention, a correlation was found between said exosomal RNA content and corresponding cellular RNA content. Therefore, analyzing exosomal RNA provides both qualitative and quantitative information about the cellular RNA content of the corresponding cells. Advantageously, this makes it possible to provide non-invasive diagnostic methods. Indeed, the analysis (whether by RNA seq, transcriptome profiling, qRT-PCR or array) is performed on a biological sample derived from body fluids, such as derived from urine, blood or cerebrospinal fluid. Such fluids are more easily and readily available than corresponding organs (bladder, heart or brain). Correspondingly, the present invention provides diagnostic methods that are non-invasive and yet reliable. In some aspects, it is envisioned to use a subpopulation of exosomes as starting material to extract RNA. This may allow the analysis of exosome subpools/subpopulations.

If reasoning that exosomes contribute to RNA transport, then exosomes could provide a delivery system in therapeutics. This would allow the delivery of a therapeutic RNA to a cell, wherein said therapeutic RNA may silence or express a gene in a cell. The present invention contemplates delivery of the exosome itself, or of an exosome-shedding cell. The delivery may occur in vivo or ex vivo. Delivery may rely on a targeting ligang. Said targeting ligand may be one or more prey exosome biomarker as described herein. For example, the prey exosome biomarker may be selected from proteins as per Table D, column G; or proteins as per Table D, column H; or proteins as per Table D, column I; or proteins as per Table D, column J; or proteins as per Table D, column K; or proteins as per Table D, column L; or proteins as per Table D, column M; the prey exosome biomarker may be FLRT3 and/or L1CAM; or efficient fragments thereof.

The term “library” as used herein generally means a multiplicity of member components constituting the library which member components individually differ with respect to at least one property, for example, a scFv library. Particularly, as will be apparent to the skilled artisan, “library” means a plurality of nucleic acids/polynucleotides, preferably in the form of vectors comprising functional elements (promoter, transcription factor binding sites, enhancer, etc.) necessary for expression of polypeptides or RNA molecules, either in vitro or in vivo, which are functionally linked to coding sequences for polypeptides or RNA molecules. The vector can be a plasmid or a viral-based vector suitable for expression in prokaryotes or eukaryotes or both, preferably for expression in mammalian cells. There should also be at least one, preferably multiple pairs of cloning sites for insertion of coding sequences into the library, and for subsequent recovery or cloning of those coding sequences. The cloning sites can be restriction endonuclease recognition sequences, or other recombination based recognition sequences such as loxP sequences for Cre recombinase, or the Gateway system (ThermoFisher, Inc.) as described in U.S. Pat. No. 5,888,732, the contents of which is incorporated by reference herein. Coding sequences for polypeptides can be cDNA, genomic DNA fragments, or random/semi-random polynucleotides. The methods for cDNA or genomic DNA library construction are well-known in the art, which can be found in a number of commonly used laboratory molecular biology manuals described herein.

In an aspect, the present invention provides for libraries of polynucleotide sequences encoding for interacting protein or RNA molecules. Methods of making libraries are well known in the art, in which the methods may use any of a variety of reverse transcriptases and optionally other DNA polymerases, vectors for cloning cDNAs, as well as adapters, linkers, restriction enzymes, and ligases or recombination enzymes for combining synthesized cDNA molecules with vectors. In some preferred embodiments of the invention, recombinational cloning is employed to insert cDNA molecules into expression vectors, and in these embodiments, adapters comprise recognition sites for recombination enzymes.

Members of a library may include any protein or RNA molecule chosen from any protein or RNA molecule of interest and includes protein or RNA molecules of unknown, known, or suspected diagnostic, therapeutic, or pharmacological importance. For example, the protein of interest can be a protein or RNA molecule suspected of being involved in a cellular process, for example, receptor signaling, apoptosis, cell proliferation, cell differentiation, immune responses or import or export of toxins and nutrients. The present invention can allow for genome wide interaction studies of key proteins expressed during these different immune cell states. As such, protein or RNA molecules of interest may be protein or RNA molecules expressed from an entire genome. Protein or RNA molecules expressed from a single cell type or from cells having a specific cell state may also be chosen.

The protein molecules of the present invention can be derived from all or a portion of a known protein or a mutant thereof, all or a portion of an unknown protein (e.g., encoded by a gene cloned from a cDNA library), or a random polypeptide sequence. Members of a DNA expression library, such as a cDNA or synthetic DNA library may be used. The full length of the protein or RNA molecule of interest, or a portion thereof, can be used. In the instance when the protein of interest is of a large size, e.g., has a molecular weight of over 20 kDa, it may be more convenient to use a portion of the protein.

Polynucleotide sequences which encode the protein or RNA molecule of interest may be inserted into a vector such that the desired protein or RNA molecule is produced in a host mammalian cell. The vectors may include a proximity detection molecule. The proximity detection molecules may be encoded in-frame with a polynucleotide sequence encoding for a protein library member. In the case of RNA molecules, the vector encoding an RNA molecule or a separate vector may encode for a fusion protein that recognizes a loop structure within the RNA molecule. In preferred embodiments, the fusion protein is encoded by a polynucleotide sequence on the same vector as the RNA molecule, such that if a cell expresses the RNA molecule it will also express the fusion protein. The fusion protein includes a proximity detection molecule, thereby allowing the RNA molecule to be bound by a proximity detection molecule after expression. Preferably, the recombinant expression vector includes one or more regulatory sequences operably linked to the polynucleotide sequence to be expressed. The term “regulatory sequence” includes promoters, enhancers and other expression control elements (e.g., polyadenylation signals).

In an exemplary embodiment, a cDNA library may be constructed from an mRNA population and inserted into an expression vector. Such a library of choice may be constructed de novo using commercially available kits or using well established preparative procedures (see, for example, Current Protocols in Molecular Biology, Eds. Ausubel et al. John Wiley & Sons: 1992). Alternatively, a number of cDNA libraries (from a number of different organisms) are publicly and commercially available. In the instance where it is preferable to replicate and store the polynucleotide sequences using a bacterial host cell, the DNA sequences are inserted into a vector which contains an appropriate origin of replication. It is also noted that protein or RNA molecules need not be naturally occurring full-length protein or RNA molecules. In certain embodiments, protein or RNA molecules can be encoded by synthetic DNA sequences.

The polynucleotide sequences encoding the desired protein or RNA molecule are typically operably linked to suitable transcriptional or translational regulatory elements. The regulatory elements typically include a transcriptional promoter, a sequence encoding suitable mRNA ribosomal binding sites, and sequences that control the termination of transcription and translation. The ability to replicate in a host, usually conferred by an origin of replication, and a selection gene to facilitate recognition of transformants may additionally be incorporated.

The nucleic acid sequences encoding the proteins or RNA molecules may be expressed in a variety of host cells, including E. coli and other bacterial hosts, and preferably eukaryotic host cells including but not limited to yeast, insect cells, and mammalian cells. The polynucleotide sequences will be operably linked to appropriate expression control sequences for each host. In a most preferred embodiment, the host cells comprise mammalian cells.

Many different mammalian cell types may be used in the practice of the invention. Cells suitable for use include primary cultures, cultures of immortalized cells or genetically manipulated strains of cells.

One of the main criteria for selection of a particular cell type may be the nature of post translational modification of target proteins expressed where the binding of such modified target proteins to a protein, RNA or small molecule may more accurately mimic the natural state. Cells that are associated with a particular disease state, or that originate from a particular tissue type may be chosen. Another criteria is the selection of a suitable cellular background to mimic the activity of a small molecule in its target tissue or cell type. If studying toxicity it may be appropriate to select a cell type associated with that toxicity, e.g. liver. Cell lines recognized in the art as easy to transfect are particularly preferred. Different mammalian cell types may also be selected according to their permeability.

Cells may also be selected on the basis of their adherence to the chosen substrate, their rate of growth, and the ease with which they can be maintained in culture. Preferably the cells are human cells.

Any cultured mammalian cell can be used in the present invention, e.g., a primary, secondary, or immortalized cell. Exemplary mammalian cells are those of mouse, hamster, rat, rabbit, dog, cow, and primate including human. They may be of a wide variety of tissue types, including mast cells, endothelial cells, hepatic cells, kidney cells, or other cell types.

As used herein, the term primary cell means cells isolated from a mammal (e.g., from a tissue source), which are grown in culture for the first time before subdivision and transfer to a subculture. The term secondary cell means cells at all subsequent steps in culturing. That is, the first time a primary cell is removed from the culture substrate and passaged, it is referred to as a secondary cell, as are all cells in subsequent passages. Examples of mammalian primary and secondary cells which can be transfected include fibro-blasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells and precursors of these somatic cell types.

Immortalized cells are cell lines that exhibit an apparently unlimited lifespan in culture. Examples of immortalized human cell lines useful for the present invention include, but are not limited to, HEK 293 cells and derivatives of HEK 293 cells (ATCC CRL 1573), HT1080 cells (ATCC CCL 121), HeLa cells and derivatives of HeLa cells (ATCC CCL 2, 2.1 and 2.2), MCF-7 breast cancer cells (ATCC HTB 22), K-562 leukemia cells (ATCC CCL 243), KB carcinoma cells (ATCC CCL 17), Raji cells (ATCC CCL 86), Jurkat cells (ATCC TIB 152), Namalwa cells (ATCC CRL 1432), HL-60 cells (ATCC CCL 240), Daudi cells (ATCC CCL 213), RPMI 8226 cells (ATCC CCL 155), U-937 cells (ATCC CRL 1593), Bowes Melanoma cells (ATCC CRL 9607), WI-38 cells (ATCC CLL 75), and MOLT-4 cells (ATCC CRL 1582).

The exosomes of the present invention may be loaded with exogenous cargoes, such as a therapeutic RNA, using electroporation protocols adapted for nanoscale applications (see, e.g., Alvarez-Erviti et al. 2011, Nat Biotechnol 29: 341). As electroporation for membrane particles at the nanometer scale is not well-characterized, nonspecific Cy5-labeled siRNA was used for the empirical optimization of the electroporation protocol. The amount of encapsulated siRNA was assayed after ultracentrifugation and lysis of exosomes. Electroporation at 400 V and 125 μF resulted in the greatest retention of siRNA and was used for all subsequent experiments.

Alvarez-Erviti et al. administered 150 μg of each BACE1 siRNA encapsulated in 150 μg of RVG exosomes to normal C57BL/6 mice and compared the knockdown efficiency to four controls: untreated mice, mice injected with RVG exosomes only, mice injected with BACE1 siRNA complexed to an in vivo cationic liposome reagent and mice injected with BACE1 siRNA complexed to RVG-9R, the RVG pep tide conjugated to 9 D-arginines that electrostatically binds to the siRNA. Cortical tissue samples were analyzed 3 d after administration and a significant protein knockdown (45%, P<0.05, versus 62%, P<0.01) in both siRNA-RVG-9R-treated and siRNARVG exosome-treated mice was observed, resulting from a significant decrease in BACE1 mRNA levels (66% [+ or −] 15%, P<0.001 and 61% [+ or −] 13% respectively, P<0.01). Moreover, Applicants demonstrated a significant decrease (55%, P<0.05) in the total [beta]-amyloid 1-42 levels, a main component of the amyloid plaques in Alzheimer's pathology, in the RVG-exosome-treated animals. The decrease observed was greater than the 3-amyloid 1-40 decrease demonstrated in normal mice after intraventricular injection of BACE1 inhibitors. Alvarez-Erviti et al. carried out 5′-rapid amplification of cDNA ends (RACE) on BACE1 cleavage product, which provided evidence of RNAi-mediated knockdown by the siRNA.

Finally, Alvarez-Erviti et al. investigated whether siRNA-RVG exosomes induced immune responses in vivo by assessing IL-6, IP-10, TNFα and IFN-α serum concentrations. Following siRNA-RVG exosome treatment, nonsignificant changes in all cytokines were registered similar to siRNA-transfection reagent treatment in contrast to siRNA-RVG-9R, which potently stimulated IL-6 secretion, confirming the immunologically inert profile of the exosome treatment. Given that exosomes encapsulate only 20% of siRNA, delivery with RVG-exosome appears to be more efficient than RVG-9R delivery as comparable mRNA knockdown and greater protein knockdown was achieved with fivefold less siRNA without the corresponding level of immune stimulation. This experiment demonstrated the therapeutic potential of RVG-exosome technology, which is potentially suited for long-term silencing of genes related to neurodegenerative diseases. The exosome delivery system of Alvarez-Erviti et al. may be applied to deliver the exosome of the present invention to therapeutic targets, especially neurodegenerative diseases. A dosage of about 100 to 1000 mg of a target RNA encapsulated in about 100 to 1000 mg of exosomes may be contemplated for the present invention.

El-Andaloussi et al. (Nature Protocols 7, 2112-2126 (2012)) discloses how exosomes derived from cultured cells can be harnessed for delivery of siRNA in vitro and in vivo. This protocol first describes the generation of targeted exosomes through transfection of an expression vector, comprising an exosomal protein fused with a peptide ligand. Next, El-Andaloussi et al. explain how to purify and characterize exosomes from transfected cell supernatant. Next, El-Andaloussi et al. detail crucial steps for loading siRNA into exosomes. Finally, El-Andaloussi et al. outline how to use exosomes to efficiently deliver siRNA in vitro and in vivo in mouse brain. Examples of anticipated results in which exosome-mediated siRNA delivery is evaluated by functional assays and imaging are also provided. The entire protocol takes ˜3 weeks. Delivery or administration according to the invention may be performed using exosomes produced from self-derived dendritic cells.

In another embodiment, the plasma exosomes of Wahlgren et al. (Nucleic Acids Research, 2012, Vol. 40, No. 17 e130) are contemplated. Exosomes are nano-sized vesicles (30-90 nm in size) produced by many cell types, including dendritic cells (DC), B cells, T cells, mast cells, epithelial cells and tumor cells. These vesicles are formed by inward budding of late endosomes and are then released to the extracellular environment upon fusion with the plasma membrane. Because exosomes naturally carry RNA between cells, this property might be useful in gene therapy.

The chemical transfection of a target RNA into exosomes may be conducted similarly to siRNA (see, e.g., Wahlgren et al. Nucleic Acids Research, 2012, Vol. 40, No. 17 e130). The exosomes may be co-cultured with monocytes and lymphocytes isolated from the peripheral blood of healthy donors. Therefore, it may be contemplated that exosomes containing a target RNA may be introduced to monocytes and lymphocytes of and autologously reintroduced into a human.

Markers are identified for a number of disorders. Such markers are useful in the diagnostic, prognostic and/or therapy of respective disorders. Such markers include disease-associated genes and polynucleotides.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.).

Examples of disease-associated genes and polynucleotides are listed in Tables A and B. Disease specific information is available from McKusick-Nathans Institute of Genetic Medicine, Johns Hopkins University (Baltimore, Md.) and National Center for Biotechnology Information, National Library of Medicine (Bethesda, Md.), available on the World Wide Web. Examples of signaling biochemical pathway-associated genes and polynucleotides are listed in Table C. Mutations in these genes and pathways can result in production of improper proteins or proteins in improper amounts which affect function.

TABLE A

| DISEASE/DISORDERS | GENE(S) |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat | HTT (Huntington's Dx); SBMA/SMAX1/AR (Kennedy's |
| Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE B

| | |
|---|---|
| Blood and | Anemia (CDAN1, CDA1, RPS19, DBA, PKLR, PK1, NT5C3, UMPH1, |
| coagulation diseases | PSN1, RHAG, RH50A, NRAMP2, SPTB, ALAS2, ANH1, ASB, |
| and disorders | ABCB7, ABC7, ASAT); Bare lymphocyte syndrome (TAPBP, TPSN, |
| | TAP2, ABCB3, PSF2, RING11, MHC2TA, C2TA, RFX5, RFXAP, |
| | RFX5), Bleeding disorders (TBXA2R, P2RX1, P2X1); Factor H and |
| | factor H-like 1 (HF1, CFH, HUS); Factor V and factor VIII (MCFD2); |
| | Factor VII deficiency (F7); Factor X deficiency (F10); Factor XI |
| | deficiency (F11); Factor XII deficiency (F12, HAF); Factor XIIIA |
| | deficiency (F13A1, F13A); Factor XIIIB deficiency (F13B); Fanconi |
| | anemia (FANCA, FACA, FA1, FA, FAA, FAAP95, FAAP90, FLJ34064, |
| | FANCB, FANCC, FACC, BRCA2, FANCD1, FANCD2, FANCD, |
| | FACD, FAD, FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A (F8, F8C, |
| | HEMA); Hemophilia B (F9, HEMB), Hemorrhagic disorders (PI, ATT, |
| | F5); Leukocyde deficiencies and disorders (ITGB2, CD18, LCAMB, |
| | LAD, EIF2B1, EIF2BA, EIF2B2, EIF2B3, EIF2B5, LVWM, CACH, |
| | CLE, EIF2B4); Sickle cell anemia (HBB); Thalassemia (HBA2, HBB, |
| | HBD, LCRB, HBA1). |
| Cell dysregulation | B-cell non-Hodgkin lymphoma (BCL7A, BCL7); Leukemia (TAL1, |
| and oncology | TCL5, SCL, TAL2, FLT3, NBS1, NBS, ZNFN1A1, IK1, LYF1, |
| diseases and disorders | HOXD4, HOX4B, BCR, CML, PHL, ALL, ARNT, KRAS2, RASK2, |
| | GMPS, AF10, ARHGEF12, LARG, KIAA0382, CALM, CLTH, |
| | CEBPA, CEBP, CHIC2, BTL, FLT3, KIT, PBT, LPP, NPM1, NUP214, |
| | D9S46E, CAN, CAIN, RUNX1, CBFA2, AML1, WHSC1L1, NSD3, |
| | FLT3, AF1Q, NPM1, NUMA1, ZNF145, PLZF, PML, MYL, STAT5B, |
| | AF10, CALM, CLTH, ARL11, ARLTS1, P2RX7, P2X7, BCR, CML, |
| | PHL, ALL, GRAF, NF1, VRNF, WSS, NFNS, PTPN11, PTP2C, SHP2, |
| | NS1, BCL2, CCND1, PRAD1, BCL1, TCRA, GATA1, GF1, ERYF1, |
| | NFE1, ABL1, NQO1, DIA4, NMOR1, NUP214, D9S46E, CAN, CAIN). |
| Inflammation and | AIDS (KIR3DL1, NKAT3, NKB1, AMB11, KIR3DS1, IFNG, CXCL12, |
| immune related | SDF1); Autoimmune lymphoproliferative syndrome (TNFRSF6, APT1, |
| diseases and disorders | FAS, CD95, ALPS1A); Combined immunodeficiency, (IL2RG, |
| | SCIDX1, SCIDX, IMD4); HIV-1 (CCL5, SCYA5, D17S136E, TCP228), |
| | HIV susceptibility or infection (IL10, CSIF, CMKBR2, CCR2, |
| | CMKBR5, CCCKR5 (CCR5)); Immunodeficiencies (CD3E, CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, PIDX, |
| | TNFRSF14B, TACI); Inflammation (IL-10, IL-1 (IL-1a, IL-1b), IL-13, |
| | IL-17 (IL-17a (CTLA8), IL-17b, IL-17c, IL-17d, IL-17f), II-23, Cx3cr1, |
| | ptpn22, TNFa, NOD2/CARD15 for IBD, IL-6, IL-12 (IL-12a, IL-12b), |
| | CTLA4, Cx3cl1); Severe combined immunodeficiencies (SCIDs)(JAK3, |
| | JAKL, DCLRE1C, ARTEMIS, SCIDA, RAG1, RAG2, ADA, PTPRC, |
| | CD45, LCA, IL7R, CD3D, T3D, IL2RG, SCIDX1, SCIDX, IMD4). |
| Metabolic, liver, | Amyloid neuropathy (TTR, PALB); Amyloidosis (APOA1, APP, AAA, |
| kidney and protein | CVAP, AD1, GSN, FGA, LYZ, TTR, PALB); Cirrhosis (KRT18, KRT8, |
| diseases and disorders | CIRH1A, NAIC, TEX292, KIAA1988); Cystic fibrosis (CFTR, ABCC7, |
| | CF, MRP7); Glycogen storage diseases (SLC2A2, GLUT2, G6PC, |
| | G6PT, G6PT1, GAA, LAMP2, LAMPB, AGL, GDE, GBE1, GYS2, |
| | PYGL, PFKM); Hepatic adenoma, 142330 (TCF1, HNF1A, MODY3), |
| | Hepatic failure, early onset, and neurologic disorder (SCOD1, SCO1), |
| | Hepatic lipase deficiency (LIPC), Hepatoblastoma, cancer and |
| | carcinomas (CTNNB1, PDGFRL, PDGRL, PRLTS, AXIN1, AXIN, |
| | CTNNB1, TP53, P53, LFS1, IGF2R, MPRI, MET, CASP8, MCH5; |
| | Medullary cystic kidney disease (UMOD, HNFJ, FJHN, MCKD2, |
| | ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/Skeletal | Becker muscular dystrophy (DMD, BMD, MYF6), Duchenne Muscular |
| diseases and disorders | Dystrophy (DMD, BMD); Emery-Dreifuss muscular dystrophy (LMNA, |
| | LMN1, EMD2, FPLD, CMD1A, HGPS, LGMD1B, LMNA, LMN1, |
| | EMD2, FPLD, CMD1A); Facioscapulohumeral muscular dystrophy |
| | (FSHMD1A, FSHD1A); Muscular dystrophy (FKRP, MDC1C, |
| | LGMD2I, LAMA2, LAMM, LARGE, KIAA0609, MDC1D, FCMD, |
| | TTID, MYOT, CAPN3, CANP3, DYSF, LGMD2B, SGCG, LGMD2C, |
| | DMDA1, SCG3, SGCA, ADL, DAG2, LGMD2D, DMDA2, SGCB, |
| | LGMD2E, SGCD, SGD, LGMD2F, CMD1L, TCAP, LGMD2G, |
| | CMD1N, TRIM32, HT2A, LGMD2H, FKRP, MDC1C, LGMD2I, TTN, |
| | CMD1G, TMD, LGMD2J, POMT1, CAV3, LGMD1C, SEPN1, SELN, |
| | RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, |
| | TIRC7, OC116, OPTB1); Muscular atrophy (VAPB, VAPC, ALS8, |
| | SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological and | ALS (SOD1, ALS2, STEX, FUS, TARDBP, VEGF (VEGF-a, VEGF-b, |
| neuronal diseases | VEGF-c); Alzheimer disease (APP, AAA, CVAP, AD1, APOE, AD2, |
| and disorders | PSEN2, AD4, STM2, APBB2, FE65L1, NOS3, PLAU, URK, ACE, |
| | DCP1, ACE1, MPO, PACIP1, PAXIP1L, PTIP, A2M, BLMH, BMH, |
| | PSEN1, AD3); Autism (Mecp2, BZRAP1, MDGA2, Sema5A, Neurexin |
| | 1, GLO1, MECP2, RTT, PPMX, MRX16, MRX79, NLGN3, NLGN4, |
| | KIAA1260, AUTSX2); Fragile X Syndrome (FMR2, FXR1, FXR2, |
| | mGLUR5); Huntington's disease and disease like disorders (HD, IT15, |
| | PRNP, PRIP, JPH3, JP3, HDL2, TBP, SCA17); Parkinson disease |
| | (NR4A2, NURR1, NOT, TINUR, SNCAIP, TBP, SCA17, SNCA, |
| | NACP, PARK1, PARK4, DJ1, PARK7, LRRK2, PARK8, PINK1, |
| | PARK6, UCHL1, PARK5, SNCA, NACP, PARK1, PARK4, PRKN, |
| | PARK2, PDJ, DBH, NDUFV2); Rett syndrome (MECP2, RTT, PPMX, |
| | MRX16, MRX79, CDKL5, STK9, MECP2, RTT, PPMX, MRX16, |
| | MRX79, x-Synuclein, DJ-1); Schizophrenia (Neuregulin1 (Nrg1), Erb4 |
| | (receptor for Neuregulin), Complexin1 (Cplx1), Tph1 Tryptophan |
| | hydroxylase, Tph2, Tryptophan hydroxylase 2, Neurexin 1, GSK3, |
| | GSK3a, GSK3b, 5-HTT (Slc6a4), COMT, DRD (Drd1a), SLC6A3, |
| | DAOA, DTNBP1, Dao (Dao1)); Secretase Related Disorders (APH-1 |
| | (alpha and beta), Presenilin (Psen1), nicastrin, (Ncstn), PEN-2, Nos1, |
| | Parp1, Nat1, Nat2); Trinucleotide Repeat Disorders (HTT (Huntington's |
| | Dx), SBMA/SMAX1/AR (Kennedy's Dx), FXN/X25 (Friedrich's |
| | Ataxia), ATX3 (Machado- Joseph's Dx), ATXN1 and ATXN2 |
| | (spinocerebellar ataxias), DMPK (myotonic dystrophy), Atrophin-1 and |
| | Atn1 (DRPLA Dx), CBP (Creb-BP - global instability), VLDLR |
| | (Alzheimer's), Atxn7, Atxn10). |
| Occular diseases | Age-related macular degeneration (Abcr, Ccl2, Cc2, cp (ceruloplasmin), |
| and disorders | Timp3, cathepsinD, Vldlr, Ccr2); Cataract (CRYAA, CRYA1, CRYBB2, |
| | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, |
| | CRYGD, CRYG4, BFSP2, CP49, CP47, HSF4, CTM, HSF4, CTM, |
| | MIP, AQP0, CRYAB, CRYA2, CTPP2, CRYBB1, CRYGD, CRYG4, |
| | CRYBB2, CRYB2, CRYGC, CRYG3, CCL, CRYAA, CRYA1, GJA8, |
| | CX50, CAE1, GJA3, CX46, CZP3, CAE3, CCM1, CAM, KRIT1); |
| | Corneal clouding and dystrophy (APOA1, TGFBI, CSD2, CDGG1, |
| | CSD, BIGH3, CDG2, TACSTD2, TROP2, M1S1, VSX1, RINX, PPCD, |
| | PPD, KTCN, COL8A2, FECD, PPCD2, PIP5K3, CFD); Cornea plana |
| | congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, |
| | NTG, NPG, CYP1B1, GLC3A); Leber congenital amaurosis (CRB1, |
| | RP12, CRX, CORD2, CRD, RPGRIP1, LCA6, CORD9, RPE65, RP20, |
| | AIPL1, LCA4, GUCY2D, GUC2D, LCA1, CORD6, RDH12, LCA3); |
| | Macular dystrophy (ELOVL4, ADMD, STGD2, STGD3, RDS, RP7, |
| | PRPH2, PRPH, AVMD, AOFMD, VMD2). |

TABLE C

| CELLULAR | |
|---|---|
| FUNCTION | GENES |
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance | PRKCE; ITGAM; ROCK1; ITGA5; CXCR4; ADAM12; |
| Signaling | IGF1; RAC1; RAP1A; E1F4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor | PRKCE; ITGAM; ROCK1; ITGA5; CXCR4; IRAK1; |
| Signaling | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKC1; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor | RAC1; PTEN; LYN; ELK1; MAPK1; RAC2; PTPN11; |
| Signaling | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon | HSPB1; EP300; FASN; TGM2; RXRA; MAPK1; NQO1; |
| Receptor | NCOR2; SP1; ARNT; CDKN1B; FOS; CHEK1; |
| Signaling | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ: TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4: PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5: PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2: ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| Signaling | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2: MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated | PRKCE; EP300; SOD2; PRKCZ; MAPK1; SQSTM1; |
| Oxidative | NQO1; PIK3CA; PRKCI; FOS; PIK3CB; PIK3C3; MAPK8; |
| Stress Response | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell | PRKCE; RAC1; PRKCZ; MAPK1; RAC2; PTPN11; |
| Signaling | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; |
| Signaling | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; |
| Nicotinamide | PLK1; AKT2; CDK8; MAPK8; MAPK3; PRKCD; PRKAA1; |
| Metabolism | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKC1; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor | IRAK1; EIF2AK2; MYD88; TRAF6; PPARA; ELK1; |
| Signaling | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK | NTRK2; MAPK1; PTPN11; PIK3CA; CREB1; FOS; |
| Signaling | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated | IRAK1; MYD88; TRAF6; PPARA; RXRA; ABCA1; |
| Inhibition | MAPK8; ALDH1A1; GSTP1; MAPK9; ABCB1; TRAF2; |
| of RXR Function | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in | KDR; FLT1; PGF; AKT2; PIK3CA; PIK3CB; PIK3C3; |
| the Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated | RAP1A; MAPK1; GNAS; CREB1; CAMK2A; MAPK3; |
| Signaling | SRC; RAF1; MAP2K2; STAT3; MAP2K1; BRAF; ATF4 |
| Mitochondrial | SOD2; MAPK8; CASP8; MAPK10; MAPK9; CASP9; |
| Dysfunction | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta | GNAS; GNAQ; PPP2R1A; GNB2L1; PPP2CA; PPP1CC; |
| Adrenergic | PPP2R5C |
| Signaling | |
| Glycolysis/ | HK2; GCK; GPI; ALDH1A1; PKM2; LDHA; HK1 |
| Gluconeogenesis | |
| Interferon Signaling | IRF1; SOCS1; JAK1; JAK2; IFITM1; STAT1; IFIT3 |
| Sonic Hedgehog | ARRB2; SMO; GLI2; DYRK1A; GLI1; GSK3B; DYRK1B |
| Signaling | |
| Glycerophospholipid | PLD1; GRN; GPAM; YWHAZ; SPHK1; SPHK2 |
| Metabolism | |
| Phospholipid | PRDX6; PLD1; GRN; YWHAZ; SPHK1; SPHK2 |
| Degradation | |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision | ERCC5; ERCC4; XPA; XPC; ERCC1 |
| Repair Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm | CSNK1E; CREB1; ATF4; NR1D1 |
| Signaling | |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of | GSTP1; CYP1B1 |
| Xenobiotics | |
| by Cytochrome p450 | |
| Methane Metabolism | PRDX6; PRDX1 |
| Phenylalanine | PRDX6; PRDX1 |
| Metabolism | |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated | PRDX1 |
| Oxidative | |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental | BMP-4; Chordin (Chrd); Noggin (Nog); WNT (Wnt2; |
| Neurology | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4fl or Brn3a); Numb; Reln |

TABLE D

| | | | | | F | |
|---|---|---|---|---|---|---|
| | | | | | CSF | |
| | | | D | E | transmembrane | |
| A | | | CSF | CSF | proteins | |
| CSF mass | | | transmembrane | transmembrane | that are | |
| spec | B | C | proteins | proteins | neuron | G |
| transmembrane | neuron | neuron | that are | that are | specific in | Proteins |
| protein | specific | specific | neuron | neuron | mouse | found in |
| gene | genes in | genes in | specific in | specific in | and | neuron |
| names | mouse | human | mouse | human | human | exosomes |
| APP | HTR7 | GAD2 | CACNA2D2 | PCDHGC4 | KIT | ABCA3 |
| A4 | NT5C1A | PCDHGC4 | L1CAM | CNR1 | LINGO2 | ABCD2 |
| AD1 | YWHAG | SYNPR | NDST4 | KIT | EPHA7 | ACVR1B |
| SLC3A2 | CHRNB2 | CNR1 | AJAP1 | SYT1 | GPR158 | ACVR2A |
| MDU1 | ACBD7 | DLX6-AS1 | EPHA8 | CDH9 | RTN1 | ANKAR |
| ABCA2 | HTR1B | RELN | WSCD2 | LINGO2 | CDH7 | APLP1 |
| ABC2 | HTR1DB | GABRA1 | KCNS2 | ROBO2 | WSCD2 | APLP2 |
| KIAA1062 | HTR3A | KCNC2 | CDH4 | EPHA7 | CDH18 | APMAP |
| ACE | 5HT3R | VIP | CACNA2D1 | PCDH8 | NDST4 | ARL6IP5 |
| DCP | HTR3 | DLX1 | CACNA2D3 | PARM1 | KCNS2 | ASIC1 |
| DCP1 | CHRM3 | CCK | CDH7 | GPR158 | PLXNA4 | ATP11C |
| ADAM23 | CHRM4 | PTHLH | EPHA7 | PTPRR | SCN3B | ATP2B1 |
| MDC3 | ABCC8 | TAC3 | D130043K22RIK | RTN1 | FLRT3 | ATP2B2 |
| ABCA1 | HRINS | TAC1 | CNTNAP5A | ST8SIA3 | L1CAM | ATP2B3 |
| ABC1 | SUR | ZMAT4 | NPTXR | C11orf87 | PLD5 | ATP2B4 |
| CERP | SUR1 | CALB2 | HCN3 | HCN1 | HS6ST2 | ATP2C1 |
| ADAM10 | ABCG4 | PENK | CNTNAP5C | PTPRT | CACNA2D3 | ATP6AP1 |
| KUZ | WHITE2 | GABRG2 | SCN3B | CDH7 | HCN3 | ATP6V0A1 |
| MADM | AFF3 | NXPH2 | AI593442 | WBSCR17 | CACNA2D1 | ATP8A1 |
| ADAM15 | LAF4 | GAD1 | KIT | C9orf4 | CLSTN3 | ATP8A2 |
| MDC15 | HTR1D | RAB3C | CALY | NETO1 | AJAP1 | ATP9A |
| ADAM17 | HTR1DA | CRH | CLSTN3 | SLITRK4 | CALY | ATRN |
| CSVP | HTRL | KIT | LINGO2 | EPHA5 | SCN2B | BMPR2 |
| TACE | HTR1F | PCP4L1 | CDH18 | NRXN3 | MET | C5orf42 |
| ADGRL2 | HTR1EL | OPRK1 | CXADR | WSCD2 | | CACNA2D1 |
| KIAA0786 | HTR2C | SERTM1 | FLRT3 | SLC12A5 | | CADM1 |
| LEC1 | HTR1C | GABRB2 | MET | CDH18 | | CADM3 |
| LPHH1 | ACHE | CHRNA6 | NPCD | D4S234E | | CANX |
| LPHN2 | FAM132A | GRIN3A | HS6ST2 | NDST4 | | CD151 |
| ACVR1B | C1QDC2 | SYT4 | SCN2B | TRHDE | | CD47 |
| ACVRLK4 | CTRP12 | ENTPD3 | STX1B | KCNS2 | | CDH2 |
| ALK4 | CHRM5 | ELAVL2 | SLC24A2 | SV2A | | CDH4 |
| ADAM22 | MLLT11 | SYT1 | RTN1 | EFNB3 | | CELSR2 |
| MDC2 | AF1Q | C8orf34 | GPR158 | NOV | | CELSR3 |
| ADGRB2 | ACTN2 | GRPR | RAMP3 | CNTNAP2 | | CISD2 |
| BAI2 | ADRA2C | ZNF385D | PODXL2 | ATP1B1 | | CLCN3 |
| ADGRL1 | ADRA2L2 | CBLN4 | PRRT3 | PLXNA4 | | CLCN6 |
| KIAA0821 | ADRA2RL2 | NDNF | EFNB2 | CLSTN2 | | CLDND1 |
| LEC2 | NPPC | GRIK1-AS2 | CHL1 | CDH8 | | CPD |
| LPBN1 | CNP2 | DLX6 | D430041D05RIK | SCN3B | | CXCR4 |
| ADAM8 | AFF2 | PNOC | PLD5 | ST8SIA5 | | CYB5A |
| MS2 | FMR2 | SCG2 | CELSR2 | SEZ6 | | CYB5B |
| ADAM11 | OX19 | SLC32A1 | PGRMC1 | RYR2 | | DCHS1 |
| MDC | AJAP1 | PLCXD3 | PLXNA4 | HS6ST3 | | DISP2 |
| ADAM28 | MOT8 | CKMT1B | | FLRT3 | | EEF1E1 |
| ADAM23 | SHREW1 | CALB1 | | SCN3A | | EGFR |
| MDCL | AMY1A | CDH9 | | C11orf41 | | EPHA3 |
| ADAM9 | AMY1; | CCNA1 | | SORCS3 | | EPHA4 |
| KIAA0021 | AMY1B | RGS8 | | HMP19 | | EPHX1 |
| MCMP | AMY1; | SV2C | | L1CAM | | ESYT1 |
| MDC9 | AMY1C | RBM24 | | LPHN2 | | FAM171A1 |
| MLTNG | AMY1 | PCDHA1 | | CD200 | | FAM171A2 |
| ADGRB3 | PRMT8 | VWC2L | | PLD5 | | FDFT1 |
| BAI3 | HRMT1L3 | GLRA2 | | SORCS1 | | FER1L5 |
| KIAA0550 | HRMT1L4 | TACR1 | | GRIA4 | | FLRT3 |
| ADGRL3 | ANKRD24 | LINGO2 | | DPP6 | | FLT1 |
| KIAA0768 | KIAA1981 | NPY | | HS6ST2 | | FOCAD |
| LEC3 | AMIGO1 | CKMT1A | | ODZ1 | | FXYD6 |
| LPHN3 | ALI2 | ZCCHC12 | | CNTNAP5 | | GRIK2 |
| AJAP1 | AMIGO | LNP1 | | SLITRK3 | | GRIK3 |
| MOT8 | KIAA1163 | SLC10A4 | | LRFN5 | | GUCY2D |
| SHREW1 | ANKRD45 | NPR3 | | LRRTM2 | | HECTD4 |
| PAM | AP3B2 | CAMK1G | | EPHA10 | | HECTD4 |
| ANO4 | ARG2 | ROBO2 | | FXYD6 | | HSD17B12 |
| TMEM16D | ARMC2 | KCNA3 | | PLXNC1 | | IGSF3 |
| ANTXR1 | ATP2B3 | LOC147670 | | CACNA2D3 | | ISLR2 |
| ATR | ARX | SYNGR3 | | ADAM22 | | ITFG1 |
| TEM8 | ASGR1 | GABRG3 | | PAM | | ITGA7 |
| ANPEP | CLEC4H1 | KCNJ3 | | SLITRK5 | | ITGA9 |
| APN | ASPHD1 | MAP7D2 | | C10orf35 | | ITGAM |
| CD13 | ATG9B | GRIN2B | | ATRNL1 | | ITM2B |
| PEPN | APG9L2 | SRRM4 | | ATP1A3 | | ITM2B |
| APLP2 | NOS3AS | KCNQ5 | | ST8SIA2 | | ITM2C |
| APPL2 | ATP2B2 | OLFM3 | | HCN3 | | ITPR1 |
| APMAP | PMCA2 | KRT222 | | CCDC136 | | JAM3 |
| C20orf3 | DNAJC6 | CAMKV | | CACNA2D1 | | KIAA1109 |
| UNQ1869/PRO4305 | KIAA0473 | EPHA7 | | SPINT2 | | KIAA1324L |
| AOC3 | B3GALT1 | GRIN2A | | FNDC9 | | KIDINS220 |
| VAP1 | BAIAP3 | STMN2 | | SLC8A2 | | KTN1 |
| APLP1 | KIAA0734 | SNAP25 | | NPTN | | L1CAM |
| ATF6B | BASP1 | DLX5 | | FAR2 | | LDLR |
| CREBL1 | NAP22 | INA | | TPBG | | LINGO1 |
| G13 | ADAMTS15 | SST | | SEZ6L | | LPHN2 |
| ATP11B | BARHL2 | SCN2A | | UNC5D | | LPHN3 |
| ATPIF | ADAMTS14 | CHRM2 | | TMEM132D | | LPPR3 |
| ATPIR | B3GALT2 | NPAS4 | | B4GALNT1 | | LRP1 |
| KIAA0956 | BEX2 | PCSK2 | | CLSTN3 | | LRP1B |
| ATP2B1 | BHLHE22 | COCH | | ATP2B1 | | LRRC59 |
| PMCA1 | BHLHB5 | PCDH8 | | AJAP1 | | LRRC8A |
| ASTN2 | TNRC20 | BTBD11 | | B7H6 | | LRRN1 |
| KIAA0634 | BFSP1 | PARM1 | | ADAM23 | | MMP15 |
| ASTN1 | BEX4 | GPR158 | | LARGE | | NALCN |
| ASTN | BEXL1 | PVALB | | ERBB4 | | NCAM1 |
| KIAA0289 | NADE3 | MEG3 | | CALY | | NCKAP1 |
| ATF6 | BAIAP2L2 | PDYN | | PTPRN2 | | NEO1 |
| ASPH | UNQ9336/PRO34007 | SVOP | | LRP11 | | NFASC |
| BAH | BMP5 | HSPB3 | | TRPC3 | | NGFR |
| ATP1A3 | BMP6 | PTPRR | | SLC9A7 | | NPDC1 |
| ATP1A1 | VGR | CHRNB3 | | DNER | | NPR2 |
| ATP1A2 | CACNA2D1 | UCHL1 | | EPHA6 | | NRP1 |
| KIAA0778 | CACNL2A | SIAH3 | | PVR | | NRXN1 |
| ATP1B2 | CCHL2A | SLC4A10 | | SCN2B | | NRXN2 |
| B3GNT2 | MHS3 | NELL2 | | ATP1A1 | | NSDHL |
| B3GALT7 | CACNA2D3 | RTN1 | | B3GALNT1 | | NSG1 |
| B3GNT1 | CACNA1I | LGI2 | | GALNT13 | | NTRK2 |
| BACE1 | KIAA1120 | PGM2L1 | | CHST15 | | P2RX3 |
| BACE | CDH4 | SCGB2A1 | | FLT3 | | PCDHB10 |
| KIAA1149 | CDH7 | ST8SIA3 | | DCC | | PCDHB2 |
| ATP1B1 | CDH7L1 | C11orf87 | | SLITRK1 | | PCDHB5 |
| ATP1B | BEGAIN | GALNTL6 | | NAGPA | | PCDHGA11 |
| ATRN | KIAA1446 | LOC157627 | | SCAMP1 | | PCDHGB2 |
| KIAA0548 | BEND6 | GLS2 | | FAM20B | | PCDHGB6 |
| MGCA | C6orf65 | MYT1L | | RNF150 | | PCLO |
| B4GALT1 | BSN | GRIP1 | | MET | | PGRMC1 |
| GGTB2 | KIAA0434 | SYT10 | | IL1RAPL1 | | PIEZO2 |
| B3GNT4 | ZNF231 | SYTL5 | | EPHA4 | | PLD3 |
| UNQ1898/PRO4344 | BTBD11 | CCDC85A | | GRIA2 | | PLXNA1 |
| B4GALNT4 | C1QTNF1 | SYT13 | | DCBLD2 | | PLXNA2 |
| B3GAT3 | CTRP1 | HCN1 | | | | PLXNA3 |
| B4GAT1 | UNQ310/PRO353 | PTPRT | | | | PLXNA4 |
| B3GNT1 | CDH12 | CNTN5 | | | | PMEL |
| B3GNT6 | C2CD4C | SPHKAP | | | | PRAF2 |
| BSG | FAM148C | MRAP2 | | | | PSEN1 |
| UNQ6505/PRO21383 | KIAA1957 | NRIP3 | | | | PTPLAD1 |
| BCAM | NLF3 | CDH7 | | | | PTPN1 |
| LU | CABP7 | BEX5 | | | | PTPRA |
| MSK19 | CALN2 | CBLN2 | | | | PTPRD |
| BET1L | CALY | NSF | | | | PTPRF |
| GS15 | DRD1IP | DCLK1 | | | | PTPRG |
| B3GNT8 | BPIFA1 | VSTM2A | | | | PTPRO |
| B3GALT7 | LUNX | WBSCR17 | | | | PTPRS |
| BGALT15 | NASG | MTUS2 | | | | RET |
| B4GALNT1 | PLUNC | GABBR2 | | | | ROBO1 |
| GALGT | SPLUNC1 | CRHBP | | | | ROBO2 |
| SIAT2 | SPURT | LOC285878 | | | | RTN1 |
| BAI1 | UNQ787/PRO1606 | STAT4 | | | | RTN3 |
| BAMBI | CACNA2D2 | C9orf4 | | | | RTN4 |
| NMA | KIAA0558 | CHGB | | | | RTN4 |
| ATRNL1 | CBLN4 | CLVS1 | | | | SCAMP1 |
| KIAA0534 | CBLNL1 | ARL4C | | | | SCAMP3 |
| B3GALNT1 | UNQ718/PRO1382 | NPY1R | | | | SCARB2 |
| B3GALT3 | CACNA1G | HTR2C | | | | SCFD1 |
| UNQ531/PRO1074 | KIAA1123 | DIRAS2 | | | | SCN3A |
| BTN2A1 | CDH18 | RSPO3 | | | | SCN9A |
| BT2.1 | CDH14 | NETO1 | | | | SDK1 |
| BTF1 | CALCA | EGFL6 | | | | SEMA3D |
| BST2 | CALC1 | HTR5A | | | | SEMA4C |
| CACNA2D1 | CCDC152 | FSTL5 | | | | SEMA6D |
| CACNL2A | Chr5_400 | PCDH11Y | | | | SERINC1 |
| CCHL2A | CACNG5 | HOOK1 | | | | SEZ6L2 |
| MHS3 | CCK | PRR16 | | | | SLC12A2 |
| CACNA2D3 | CAMKV | LOC100144602 | | | | SLC12A7 |
| CDH20 | CASZ1 | SLITRK4 | | | | SLC22A17 |
| CDH7L3 | CST | VSNL1 | | | | SLC38A1 |
| CDH4 | SRG | FRMPD4 | | | | SLC4A10 |
| CDH6 | ZNF693 | ATP8A2 | | | | SLC4A7 |
| CDH7 | CCDC148 | EPHA5 | | | | SLC4A8 |
| CDH7L1 | CCDC6 | CAPSL | | | | SNRNP40 |
| CADM2 | D10S170 | RIMBP2 | | | | SORT1 |
| IGSF4D | TST1 | FGF13 | | | | ST6GAL1 |
| NECL3 | C4orf48 | LAMP5 | | | | STARD3NL |
| BOC | Chr4_55 | SLC2A13 | | | | STX12 |
| UNQ604/PRO1190 | CELSR2 | GRIK1 | | | | STX1B |
| CACHD1 | CDHF10 | HBG2 | | | | STX7 |
| KIAA1573 | EGFL2 | RASD2 | | | | SUSD2 |
| VWCD1 | KIAA0279 | FGF14 | | | | SYNGR1 |
| BMPR2 | MEGF3 | RASGRF1 | | | | SYP |
| PPH1 | CACNG3 | RBFOX1 | | | | SYT1 |
| CD93 | C6orf222 | MYO16 | | | | SYT11 |
| C1QR1 | CGREF1 | CPLX2 | | | | SYT2 |
| MXRA4 | CGR11 | SYN2 | | | | SYT5 |
| CDH11 | CBLN1 | GRIA1 | | | | TENM2 |
| CDH15 | CCDC116 | SCN1A | | | | TENM3 |
| CDH14 | CEACAM16 | NRXN3 | | | | TENM4 |
| CDH3 | CEAL2 | KCNJ6 | | | | TEX10 |
| CDH2 | CELF4 | GRIK2 | | | | TGFBR1 |
| CDHN | BRUNOL4 | PTCHD4 | | | | TGOLN2 |
| NCAD | CACNG2 | FABP3 | | | | THSD7A |
| CANX | CCL27 | RAB3B | | | | TM9SF2 |
| CALY | ILC | RGS4 | | | | TM9SF3 |
| DRD1IP | SCYA27 | CDH13 | | | | TM9SF4 |
| CACNA2D2 | CELF6 | IL1RAPL2 | | | | TMEFF1 |
| KIAA0558 | BRUNOL6 | WSCD2 | | | | TMEFF2 |
| C3AR1 | C8orf34 | LINC00290 | | | | TMEM132A |
| AZ3B | CDK5R2 | ZNF804A | | | | TMEM178B |
| C3R1 | NCK5AI | SLC7A14 | | | | TMEM55B |
| HNFAG09 | CELSR3 | CYP4X1 | | | | TP53I11 |
| CADM3 | CDHF11 | NMNAT2 | | | | TSPAN15 |
| IGSF4B | EGFL1 | SCGN | | | | UBR4 |
| NECL1 | FMI1 | UBE2QL1 | | | | UNC5C |
| SYNCAM3 | KIAA0812 | GPR83 | | | | VAMP2 |
| TSLL1 | MEGF2 | SCN7A | | | | VANGL2 |
| UNQ225/PRO258 | CDR1 | GPR12 | | | | VAPB |
| CD163 | C11orf87 | RXFP1 | | | | VSIG8 |
| M130 | CEND1 | FAM19A2 | | | | WDR11 |
| CDH10 | BM88 | CACNA1E | | | | XPR1 |
| CADM1 | CHAC1 | REEP1 | | | | YIPF6 |
| IGSF4 | BOTCH | SLC12A5 | | | | |
| IGSF4A | C11orf42 | TRPC4 | | | | |
| NECL2 | CLVS2 | COL25A1 | | | | |
| SYNCAM | C6orf212 | WNT10B | | | | |
| TSLC1 | C6orf213 | SLC35F4 | | | | |
| CADM4 | RLBP1L2 | GABRA5 | | | | |
| IGSF4C | CNGA3 | MAL2 | | | | |
| NECL4 | CNCG3 | GREM2 | | | | |
| TSLL2 | CHSY3 | RGS7BP | | | | |
| CA12 | CHSY2 | CDH18 | | | | |

| | | | | | M |
|---|---|---|---|---|---|
| | | | | | CSF |
| | | | K | L | transmembrane |
| | | | CSF | CSF | proteins |
| | | | transmembrane | transmembrane | that are |
| | I | J | proteins | proteins | neuron |
| | neuron | neuron | that are | that are | specific in |
| H | specific | specific | neuron | neuron | mouse |
| CSF MS | genes in | genes in | specific in | specific in | and |
| transmembrane | mouse | human | mouse | human | human |
| proteins | AND in | AND in | AND in | AND in | AND in |
| in neuron | neuron | neuron | neuron | neuron | neuron |
| exosomes | exosome | exosome | exosome | exosome | exosome |
| ACVR1B | ATP2B2 | ATP2B1 | CACNA2D1 | SYT1 | FLRT3 |
| APLP1 | ATP2B3 | ATP2B2 | CDH4 | ROBO2 | L1CAM |
| APLP2 | CACNA2D1 | ATP2B3 | CELSR2 | RTN1 | |
| APMAP | CADM3 | ATP8A2 | FLRT3 | PLXNA4 | |
| ATP2B1 | CDH4 | CACNA2D1 | L1CAM | FLRT3 | |
| ATP6AP1 | CELSR2 | CELSR3 | PGRMC1 | SCN3A | |
| ATRN | CELSR3 | DISP2 | PLXNA4 | L1CAM | |
| BMPR2 | DISP2 | EEF1E1 | RTN1 | LPHN2 | |
| CACNA2D1 | EPHA3 | EPHA3 | STX1B | FXYD6 | |
| CADM1 | FAM171A2 | EPHA4 | | CACNA2D1 | |
| CADM3 | FLRT3 | FLRT3 | | ATP2B1 | |
| CANX | IGSF3 | FXYD6 | | SCAMP1 | |
| CDH2 | ISLR2 | GRIK2 | | EPHA4 | |
| CDH4 | L1CAM | GRIK3 | | | |
| CELSR2 | NGFR | IGSF3 | | | |
| CPD | PCLO | ITFG1 | | | |
| EGFR | PGRMC1 | ITPR1 | | | |
| EPHA4 | PLXNA4 | L1CAM | | | |
| FAM171A1 | ROBO2 | LPHN2 | | | |
| FLRT3 | RTN1 | PCLO | | | |
| FXYD6 | SCN9A | PLXNA4 | | | |
| ITGA7 | SEZ6L2 | PTPRO | | | |
| ITM2B | SLC38A1 | RET | | | |
| ITM2B | STX1B | ROBO2 | | | |
| ITM2C | SUSD2 | RTN1 | | | |
| JAM3 | SYP | RTN3 | | | |
| L1CAM | SYT1 | SCAMP1 | | | |
| LDLR | SYT2 | SCN3A | | | |
| LINGO1 | SYT5 | SCN9A | | | |
| LPHN2 | THSD7A | SERINC1 | | | |
| LPHN3 | | SLC38A1 | | | |
| LRP1 | | SLC4A10 | | | |
| LRP1B | | SLC4A8 | | | |
| LRRN1 | | SYP | | | |
| MMP15 | | SYT1 | | | |
| NCAM1 | | SYT2 | | | |
| NEO1 | | THSD7A | | | |
| NFASC | | TMEFF2 | | | |
| NPDC1 | | | | | |
| NRP1 | | | | | |
| NRXN1 | | | | | |
| NRXN2 | | | | | |
| NSG1 | | | | | |
| NTRK2 | | | | | |
| PGRMC1 | | | | | |
| PLD3 | | | | | |
| PLXNA1 | | | | | |
| PLXNA3 | | | | | |
| PLXNA4 | | | | | |
| PTPLAD1 | | | | | |
| PTPRD | | | | | |
| PTPRF | | | | | |
| PTPRG | | | | | |
| PTPRS | | | | | |
| ROBO1 | | | | | |
| ROBO2 | | | | | |
| RTN1 | | | | | |
| RTN4 | | | | | |
| RTN4 | | | | | |
| SCAMP1 | | | | | |
| SCN3A | | | | | |
| SDK1 | | | | | |
| SEMA4C | | | | | |
| SEMA6D | | | | | |
| SEZ6L2 | | | | | |
| SLC12A2 | | | | | |
| SORT1 | | | | | |
| STX12 | | | | | |
| STX1B | | | | | |
| STX7 | | | | | |
| SYT1 | | | | | |
| SYT11 | | | | | |
| TENM2 | | | | | |
| TENM4 | | | | | |
| TGOLN2 | | | | | |
| TM9SF3 | | | | | |
| TM9SF4 | | | | | |
| TMEM132A | | | | | |
| UNC5C | | | | | |
| VAMP2 | | | | | |

CSF mass spec transmembrane protein gene names No. 209 onwards

| 209-243 | 244-278 | 279-314 | 315-349 | 350-384 | 385-419 |
|---|---|---|---|---|---|
| CA14 | CDHF10 | MIC2X | COLEC12 | NGC | DBH |
| UNQ690/PRO1335 | EGFL2 | MIC2Y | CLP1 | CSPG4 | DLK2 |
| CDH18 | KIAA0279 | CELSR1 | NSR2 | MCSP | EGFL9 |
| CDH14 | MEGF3 | CDHF9 | SCARA4 | GJA9 | UNQ2903/PRO28633 |
| CDH5 | CASC4 | FMI2 | SRCL | GJA10 | DPP10 |
| CDH8 | UNQ2573/PRO6308 | CHST1 | CNR1 | CXADR | DPRP3 |
| CCR1 | CD276 | CHST8 | CNR | CAR | KIAA1492 |
| CMKBR1 | B7H3 | CLIC6 | CRB2 | CLSTN2 | DSG3 |
| CMKR1 | PSEC0249 | CLIC1L | CRELD1 | CS2 | CDHF6 |
| SCYAR1 | UNQ309/PRO352 | CLMP | CIRRIN | GJA1 | DPP6 |
| CDH9 | CD81 | ACAM | UNQ188/PRO214 | GJAL | DNER |
| CANT1 | TAPA1 | ASAM | CSF1R | CXCL16 | BET |
| SHAPY | TSPAN28 | UNQ318/PRO363 | FMS | SCYB16 | UNQ262/PRO299 |
| CD44 | CD320 | C11orf87 | CSF2RA | SRPSOX | SLC1A2 |
| LHR | 8D6A | CHST10 | CSF2R | UNQ2759/PRO6714 | EAAT2 |
| MDU2 | UNQ198/PRO224 | CMTM1 | CSF2RY | DAG1 | GLT1 |
| MDU3 | CCDC136 | CKLFSF1 | COL23A1 | DCC | DRD3 |
| MIC4 | KIAA1793 | CLCC1 | CSF1 | IGDCC1 | DSG2 |
| CDHR1 | NAG6 | KIAA0761 | CCSMST1 | DCBLD2 | CDHF5 |
| KIAA1775 | CD302 | MCLC | C16orf91 | CLCP1 | DUOX2 |
| PCDH21 | CLEC13A | CHST14 | CLSTN1 | ESDN | LNOX2 |
| PRCAD | DCL1 | D4ST1 | CS1 | DDR1 | THOX2 |
| CPD | KIAA0022 | UNQ1925/PRO4400 | KIAA0911 | CAK | DSC2 |
| CCR10 | ALCAM | CHST3 | CYP26C1 | EDDR1 | CDHF2 |
| GPR2 | MEMD | CLIC1 | CRIM1 | NEP | DSC3 |
| CD248 | CHST12 | G6 | S52 | NTRK4 | DSC3 |
| CD164L1 | UNQ500/PRO1017 | NCC27 | UNQ1886/PRO4330 | PTK3A | CDHF3 |
| TEM1 | CHST15 | C9 | CSMD2 | RTK6 | DSC4 |
| CD6 | BRAG | C14orf37 | KIAA1884 | TRKE | DSCAM |
| CD9 | GALNAC4S6ST | CNTNAP2 | CLSTN3 | DDR2 | ECE1 |
| MIC3 | KIAA0598 | CASPR2 | CS3 | NTRKR3 | EFNB1 |
| TSPAN29 | C10orf35 | KIAA0868 | KIAA0726 | TKT | EFL3 |
| GIG2 | CD99 | CNTNAP5 | CSPG5 | TYRO10 | EPLG2 |
| CELSR2 | MIC2 | CASPR5 | CALEB | DCBLD1 | LERK2 |

CSF mass spec transmembrane protein gene names No. 209 onwards

| 420-454 | 455-489 | 490-524 | 525-559 | 560-594 | 595-629 |
|---|---|---|---|---|---|
| EFNB2 | ERAP1 | EPHB2 | C10orf38 | FGFBR | GGT7 |
| EPLG5 | APPILS | DRT | FAM198B | FLG | GGTL3 |
| HTKL | ARTS1 | EPHT3 | C4orf18 | FLT2 | GGTL5 |
| LERK5 | KIAA0525 | EPTH3 | ENED | HBGFR | FZD3 |
| EDNRB | UNQ584/PRO1154 | ERK | AD021 | FKRP | GAL3ST3 |
| ETRB | ERAP2 | HEK5 | UNQ2512/PRO6001 | FLT3 | GINM1 |
| EMC10 | LRAP | TYRO5 | FAM174A | CD135 | C6orf72 |
| C19orf63 | ERBB3 | EPHB3 | NS5ATP6 | FLK2 | UNQ710/PRO1361 |
| HSM1 | HER3 | ETK2 | TMEM157 | STK1 | GALNT2 |
| INM02 | ESAM | HEK2 | UNQ1912/PRO4371 | FGFR3 | GALNT11 |
| UNQ764/PRO1556 | UNQ220/PRO246 | TYRO6 | FAR2 | JTK4 | GALNT16 |
| EFNB3 | EGFR | GPR37L1 | MLSTD1 | FLRT2 | GALNTL1 |
| EPLG8 | ERBB | ETBRLP2 | FCGR2A | KIAA0405 | KIAA1130 |
| LERK8 | ERBB1 | EXT1 | CD32 | UNQ232/PRO265 | GLT8D1 |
| EPHA5 | HER1 | FAM134A | FCG2 | FLRT1 | GALA4A |
| BSK | ENPP5 | C2orf17 | FCGR2A1 | UNQ752/PRO1483 | AD-017 |
| EHK1 | UNQ550/PRO1107 | EPHA4 | IGFR2 | FNDC9 | MSTP137 |
| HEK7 | PROCR | HEK8 | FGFR2 | C5orf40 | UNQ572/PRO1134 |
| TYRO4 | EPCR | SEK | BEK | FUT11 | GLDN |
| EPHA8 | EPHA7 | TYRO1 | KGFR | FXYD6 | COLM |
| EEK | EHK3 | FAM173A | KSAM | UNQ521/PRO1056 | UNQ9339/PRO34011 |
| HEK3 | HEK11 | C16orf24 | FLRT3 | GALNT7 | GYPC |
| KIAA1459 | EPHB6 | RJD7 | KIAA1469 | FURIN | GLPC |
| EPHB1 | ENPP4 | FAM69C | UNQ856/PRO1865 | FUR | GPC |
| ELK | KIAA0879 | C18orf51 | FAT1 | PACE | GALNT18 |
| EPHT2 | NPP4 | FCGR3A | CDHF7 | PCSK3 | GALNTL4 |
| HEK6 | ENTPD4 | CD16A | FAT | GALNT6 | GPR158 |
| NET | KIAA0392 | FCG3 | FAT2 | FREM2 | KIAA1136 |
| EPHB4 | LALP70 | FCGR3 | CDHF8 | FRRS1L | GALNT10 |
| HTK | LYSAL1 | IGFR3 | KIAA0811 | C9orf4 | GPAA1 |
| MYK1 | EPHA6 | EXT2 | MEGF1 | FZD7 | GAA1 |
| TYRO11 | EHK2 | EXTL2 | FGFR1 | GALNT1 | GALNT13 |
| ENG | HEK12 | EXTR2 | BFGFR | GGT5 | KIAA1918 |
| END | EPHA10 | FAM171A1 | CEK | GGTLA1 | WBSCR17 |

CSF mass spec transmembrane protein gene names No. 209 onwards

| 630-664 | 665-699 | 700-734 | 735-769 | 770-804 | 805-839 |
|---|---|---|---|---|---|
| GALNTL3 | GXYLT1 | HLAF | IL3RB | A2MR | A2MR |
| GOLIM4 | GLT8D3 | ICAM2 | IL5RB | APR | APR |
| GIMPC | HS6ST2 | HS2ST1 | IGSF5 | LRP8 | LRP8 |
| GOLPH4 | PSEC0092 | HS2ST | JAM4 | APOER2 | APOER2 |
| GPP130 | GRIA4 | KIAA0448 | IFNAR1 | LRRC4 | LRRC4 |
| GOLM1 | GLUR4 | DGCR2 | IFNAR | BAG | BAG |
| C9orf155 | GRID2 | IDD | IFNGR1 | NAG14 | NAG14 |
| GOLPH2 | GLURD2 | KIAA0163 | ITGB8 | UNQ554/PRO1111 | UNQ554/PRO1111 |
| PSEC0242 | HCN1 | IL1RAP | IGSF8 | LSR | LSR |
| UNQ686/PRO1326 | BCNG1 | C3orf13 | CD81P3 | LISCH | LISCH |
| GPNMB | HAVCR2 | IL1R3 | EWI2 | LY75 | LY75 |
| HGFIN | TIM3 | ICAM1 | KCT4 | CD205 | CD205 |
| NMB | TIMD3 | ICOSLG | IL6ST | CLEC13B | CLEC13B |
| UNQ1725/PRO9925 | HCN2 | B7H2 | IMPAD1 | MAN1C1 | MAN1C1 |
| GPR37 | BCNG2 | B7RP1 | IMPA3 | MAN1A3 | MAN1A3 |
| GPR56 | HEPACAM | ICOSL | KIAA0319L | MANIC | MAN1C |
| TM7LN4 | HACD3 | KIAA0653 | KIAA1837 | LMAN1 | LMAN1 |
| TM7XN1 | BIND1 | IGSF11 | PP791 | ERGIC53 | ERGIC53 |
| UNQ540/PRO1083 | PTPLAD1 | BTIGSF | IFNLR1 | F5F8D | F5F8D |
| GLG1 | HS6ST3 | CXADRL1 | IL28RA | LRRC4C | LRRC4C |
| CFR1 | HEG1 | VSIG3 | LICR2 | KIAA1580 | KIAA1580 |
| ESL1 | KIAA1237 | IGSF1 | ITM2B | NGL1 | NGL1 |
| MG160 | HBEGF | IGDC1 | BRI | UNQ292/PRO331 | UNQ292/PRO331 |
| SLC2A11 | DTR | KIAA0364 | BRI2 | SELL | SELL |
| GLUT10 | DTS | PGSF2 | JAM2 | LNHR | LNHR |
| GLUT11 | HEGFL | IMPG2 | C21orf43 | LYAM1 | LYAM1 |
| GPR179 | HCN3 | IPM200 | VEJAM | MAN2A2 | MAN2A2 |
| GPR158L | KIAA1535 | IFNAR2 | UNQ219/PRO245 | MANA2X | MANA2X |
| GPR158L1 | HLA-G | IFNABR | JAM3 | LRP1B | LRP1B |
| GPR180 | HLA-6.0 | IFNARB | UNQ859/PRO1868 | LRPDIT | LRPDIT |
| ITR | HLAG | ICAM5 | KIAA1467 | LRP5 | LRP5 |
| GP1BA | HMGCR | TLCN | ITGA2B | LR3 | LR3 |
| GR1A2 | HLA-F | TLN | GP2B | LRP7 | LRP7 |
| GLUR2 | HLA-5.4 | CSF2RB | ITGAB | MANEAL | MANEAL |

CSF mass spec transmembrane protein gene names No. 209 onwards

| 840-874 | 875-909 | 910-944 | 945-979 | 980-1,014 | 1,015-1,049 |
|---|---|---|---|---|---|
| LRFN5 | UNQ671/PRO1305 | CLEC13D | NFASC | KIAA0343 | NRP1 |
| C14orf146 | MERTK | CLEC13DL | KIAA0756 | NOTCH3 | NRP |
| SALM5 | MER | MRC1L1 | CHL1 | NRG3 | VEGF165R |
| LRIG2 | MET | MRC2 | CALL | NRXN1 | OR52A1 |
| KIAA0806 | MAG | CLEC13E | NEO1 | NRXN2 | CD200 |
| LIG2 | GMA | ENDO180 | IGDCC2 | KIAA0921 | MOX1 |
| LRRN1 | MEGF10 | KIAA0709 | NGN | NOTCH2 | MOX2 |
| KIAA1497 | KIAA1780 | UPARAP | NCAM2 | NPTXR | My033 |
| Nbla10449 | MGAT1 | MXRA7 | NCAM21 | NRXN3 | OR13C3 |
| UNQ693/PRO1338 | GGNT1 | TMAP1 | NINJ1 | C14orf60 | OGFOD3 |
| MAN2A1 | GLCT1 | MUC16 | NLGN4X | KIAA0743 | C17orf101 |
| MANA2 | GLYT1 | CA125 | KIAA1260 | NLGN3 | OSMR |
| MBOAT2 | MGAT | SLC8A2 | NLGN4 | KIAA1480 | OSMRB |
| OACT2 | MEGF8 | KIAA1087 | UNQ365/PRO701 | NL3 | P2RY14 |
| LRRTM2 | C19orf49 | NCX2 | NLGN2 | NOMO3 | GPR105 |
| KIAA0416 | EGFL4 | MXRA8 | KIAA1366 | NPTN | KIAA0001 |
| LRRN2 | KIAA0817 | NAALADL1 | NOMO1 | SDFR1 | PIK3IP1 |
| LYVE1 | MMP14 | NAALADASEL | PM5 | SDR1 | HGFL |
| CRSBP1 | MGAT5 | NAALADL | NOMO2 | NSG1 | PARM1 |
| HAR | GGNT5 | NCKAP1L | NDST1 | D4S234 | UNQ1879/PRO4322 |
| XLKD1 | MMP15 | HEM1 | HSST | NRXN2 | PCDHB15 |
| UNQ230/PRO263 | MGAT2 | SLC8A1 | HSST1 | NRP2 | PCDHGA12 |
| MAN1A2 | MOG | CNC | NDST2 | VEGF165R2 | CDH21 |
| MAN1B | MLEC | NCX1 | HSST2 | NRXN1 | FIB3 |
| MAN1B1 | KIAA0152 | MYOF | NCSTN | KIAA0578 | KIAA0588 |
| UNQ747/PRO1477 | MYRF | FER1L3 | KIAA0253 | NRXN3 | UNQ371/PRO707 |
| MANEA | C11orf9 | KIAA1207 | UNQ1874/PRO4317 | KIAA0743 | PCDH9 |
| MANSC1 | KIAA0954 | SLC24A2 | NPC1 | NSG2 | PCDHGC4 |
| LOH12CR3 | MRF | NCKX2 | NCR3LG1 | NTRK2 | PCDH8 |
| UNQ316/PRO361 | MCAM | NAGPA | B7H6 | TRKB | PCDHGC3 |
| MAN1A1 | MUC18 | NCAM1 | NDST4 | OR2AK2 | PCDH2 |
| MEGF9 | IGF2R | NCAM | HSST4 | OR2AK1P | PCNX |
| EGFL5 | MPR1 | NETO1 | NPDC1 | NTRK3 | KIAA0805 |
| KIAA0818 | MRC1 | BTCL1 | NRCAM | TRKC | KIAA0995 |

CSF mass spec transmembrane protein gene names No. 209 onwards

| 1,050-1,084 | 1,085-1,119 | 1,120-1,154 | 1,155-1,189 | 1,190-1,224 | 1,225-1,259 |
|---|---|---|---|---|---|
| PCNXL1 | PSEC0164 | PIGA | DEP1 | HVEC | RNF13 |
| PCDH10 | UNQ289/PRO328 | PKD1L3 | PTPRR | PRR1 | RZF |
| KIAA1400 | PKD1 | PLD3 | ECPTP | PVRL3 | RNF150 |
| PAPPA-AS1 | PLXNA1 | PLXDC1 | PTPRQ | PRR3 | KIAA1214 |
| DIPAS | NOV | TEM3 | PTPRS | QSOX2 | RPN2 |
| PAPPAS | PLXN1 | TEM7 | PTPRF | QSCN6L1 | ROR1 |
| PCDH17 | PLXNA3 | PLXNB3 | LAR | SOXN | NTRKR1 |
| PCDH68 | PLXN4 | KIAA1206 | PTPRZ1 | EBAG9 | ROBO2 |
| PCH68 | SEX | PLXN6 | HTPZP2 | RCAS1 | KIAA1568 |
| PCDHB14 | PLXNA4 | PLXNC1 | PTPRZ | QSOX1 | RTN1 |
| PCDHGC5 | KIAA1550 | VESPR | PTPRZ2 | QSCN6 | NSP |
| PCDH7 | PLXNA4A | PLXND1 | PTPZ | UNQ2520/PRO6013 | SLC12A2 |
| BHPCDH | PLXNA4B | KIAA0620 | PTCHD2 | RAMP3 | NKCC1 |
| PDGFRB | UNQ2820/PRO34003 | PODXL | DISP3 | REEP2 | S1PR3 |
| PDGFR | PLXNB2 | PCLP | KIAA1337 | C5orf19 | EDG3 |
| PDGFR1 | KIAA0315 | PCLP1 | PVRL2 | SGC32445 | SLC22A23 |
| PODXL2 | POMGNT1 | PRLR | HVEB | RHBDF1 | C6orf85 |
| UNQ1861/PRO3742 | MGAT1.2 | ACP2 | PRR2 | C16orf8 | SLC12A5 |
| PEAR1 | UNQ746/PRO1475 | PRRT2 | PVR | DIST1 | KCC2 |
| MEGF12 | PKD1L2 | PRRT3 | PVS | IRHOM1 | KIAA1176 |
| PILRA | KIAA1879 | UNQ5823/PRO19642 | PLXDC2 | ATP6AP2 | SLC4A4 |
| PGRMC1 | PC1L2 | PRSS8 | TEM7R | ATP6IP2 | NBC |
| HPR6.6 | PLD4 | PTPRG | UNQ2514/PRO6003 | CAPER | NBC1 |
| PGRMC | C14orf175 | PTPG | PTK7 | ELDF10 | NBCE1 |
| PIGR | UNQ2488/PRO5775 | PTPRK | CCK4 | HT028 | SCIMP |
| PROKR1 | PLD5 | PTPK | PTPRD | MSTP009 | C17orf87 |
| GPR73 | PNPLA6 | PRRT1 | PTPRM | PSEC0072 | UNQ5783/PRO16090 |
| PKR1 | NTE | C6orf31 | PTPRL1 | RIC3 | RTN4 |
| PDGFRA | FXYD1 | NG5 | PTPRN | UNQ720/PRO1385 | KIAA0886 |
| PDGFR2 | PLM | PTPRN2 | ICA3 | RFNG | NOGO |
| RHEPDGFRA | PLXNB1 | KIAA0387 | ICA512 | RHAG | My043 |
| PI16 | KIAA0407 | PTPRB | PTPRT | RH50 | SP1507 |
| CRISP9 | PLXN5 | PTPB | KIAA0283 | ROBO1 | RXFP2 |
| PSPBP | SEP | PTPRJ | PVRL1 | DUTT1 | GPR106 |

CSF mass spec transmembrane protein gene names No. 209 onwards

| 1,260-1,294 | 1,295-1,329 | 1,330-1,364 | 1,365-1,399 | 1,400-1,434 | 1,435-1,469 |
|---|---|---|---|---|---|
| GREAT | UNQ1967/PRO4499 | UNQ783/PRO1317 | SLAMF7 | KIAA1854 | C1orf9 |
| LGR8 | SDK1 | SEMA6C | CS1 | SLITL1 | CH1 |
| SLC39A6 | SLC5A5 | KIAA1869 | UNQ576/PRO1138 | UNQ9197/PRO34756 | OPT |
| LIV1 | NIS | SEMAY | SEMA4B | ST3GAL6 | SLP1 |
| ZIP6 | SDC3 | POMK | KIAA1745 | SIAT10 | STAB1 |
| RYR2 | KIAA0468 | SGK196 | SEMAC | SLITRK3 | FEEL1 |
| SEZ6L2 | SEMA4D | ST3GAL4 | UNQ749/PRO1480 | KIAA0848 | KIAA0246 |
| PSK | C9orf164 | CGS23 | SEMA4C | SGMS2 | STX1B |
| UNQ1903/PRO4349 | CD100 | NANTA3 | KIAA1739 | SMS2 | STX1B1 |
| SLC38A10 | SEMAJ | SIAT4C | SEMAI | SLITRK6 | STX1B2 |
| PP1744 | SEMA6A | STZ | UNQ5855/PRO34487 | SNPH | STX12 |
| SLC39A12 | KIAA1368 | ST8SIA3 | SLITRK1 | KIAA0374 | SUSD6 |
| ZIP12 | SEMAQ | SIAT8C | KIAA1910 | SORCS1 | DRAGO |
| SCN1B | SCAMP1 | SIRPB1 | LRRC12 | SORCS | KIAA0247 |
| SCN3B | SCAMP | SLITRK4 | UNQ233/PRO266 | SORCS3 | TMEM132A |
| KIAA1158 | SCN2B | SEL1L | SLITRK5 | KIAA1059 | HSPA5BP1 |
| SCN4A | UNQ326/PRO386 | TSA305 | KIAA0918 | SORL1 | KIAA1583 |
| SDC2 | SCN3A | UNQ128/PRO1063 | LRRC11 | C11orf32 | TMEM132B |
| HSPG1 | KIAA1356 | SELPLG | SEMA6B | SORT1 | KIAA1786 |
| SDC4 | NAC3 | SEZ6 | SEMAN | SPINT2 | KIAA1906 |
| SEZ6L | ST8SIA2 | ST6GALNAC1 | SEMAZ | HAI2 | TMEM132D |
| KIAA0927 | SIAT8B | SIAT7A | UNQ1907/PRO4353 | KOP | HBE120 |
| UNQ2542/PRO6094 | STX | UNQ543/PRO848 | SGCE | SRPRB | KIAA1944 |
| SEMA6D | ST8SIA5 | ST8SIA4 | ESG | PSEC0230 | MOLT |
| KIAA1479 | SIAT8E | PST | UNQ433/PRO840 | SORCS2 | SYT1 |
| SLC39A10 | ST6GAL2 | PST1 | SIGLEC8 | KIAA1329 | SVP65 |
| KIAA1265 | KIAA1877 | SIAT8D | SAF2 | SV2A | SYT |
| ZIP10 | SIAT2 | SIGLEC5 | SLC9A7 | KIAA0736 | TMEM132C |
| SARAF | SCN4B | CD33L2 | NHE7 | PSEC0174 | TENM2 |
| TMEM66 | SECTM1 | OBBP2 | SHISA5 | STX7 | KIAA1127 |
| XTP3 | K12 | SIGLEC9 | SCOTIN | SUSD5 | ODZ2 |
| HSPC035 | SEMA4A | UNQ668/PRO1302 | PSEC0133 | KIAA0527 | TNM2 |
| NPD003 | SEMAB | CD84 | SLITRK2 | SVOPL | TENM4 |
| PSEC0019 | SEMB | SLAMF5 | CXorf2 | SUCO | KIAA1302 |

CSF mass spec transmembrane protein gene names No. 209 onwards

| 1,470-1,504 | 1,505-1,539 | 1,540-1,574 | 1,575-1,609 | 1,610-1,644 | 1,645-1,679 |
|---|---|---|---|---|---|
| ODZ4 | TLR9 | TRPC3 | SLC14A1 | XXYLT1 | hCG_37088 |
| TNM4 | UNQ5798/PRO19605 | TRP3 | HUT11 | C3orf21 | CA14 |
| SYT11 | TMED4 | TYRO3 | JK | PSEC0251 | hCG_39384 |
| KIAA0080 | ERS25 | BYK | RACH1 | FAM20B | TNFSF13 |
| SYT9 | TMED9 | DTK | UT1 | KIAA0475 | ENTPD4 |
| TGOLN2 | GP25L2 | RSE | UTE | YIPF3 | SEMA4A |
| TGN46 | TMEM5 | SKY | VASN | C6orf109 | C3AR1 |
| TGN51 | TNFRSF18 | TIF | SLITL2 | KLIP1 | hCG_22220 |
| TMEM132E | AITR | UGT2B15 | UNQ314/ | ZP2 | EPHB4 |
| | | | PRO357/ | | |
| | | | PRO1282 | | |
| TFRC | GITR | UGT2B8 | ATP6AP1 | ZPA | hCG_20448 |
| TIE1 | UNQ319/PRO364 | TYRP1 | ATP6IP1 | ZFPL1 | SCN3A |
| TIE | TMEM25 | CAS2 | ATP6S1 | PTPRN | CCR1 |
| TGFBR3 | UNQ2531/PRO6030 | TYRP | VATPS1 | hCG_15565 | hCG_15324 |
| TENM1 | TPBG | TYRRP | XAP3 | TGFBR2 | ADAM22 |
| ODZ1 | 5T4 | AXL | VLDLR | hCG_1997782 | MERTK |
| TNM1 | TM9SF3 | UFO | WSCD1 | PTPRK | HLA-G |
| PRRG1 | SMBP | UNC5B | KIAA0523 | CDH11 | MOG |
| PRGP1 | UNQ245/PRO282 | P53RDL1 | CX3CL1 | hCG_26636 | hCG_25629 |
| TMG1 | TM9SF4 | UNC5H2 | FKN | EXTL2 | HLA-G |
| TGFBR2 | KIAA0255 | UNQ1883/PRO4326 | NTT | hCG_32848 | hCG_1999524 |
| TLR1 | TMED3 | UNC5C | SCYD1 | SLC12A2 | HLA-G2.2 |
| KIAA0012 | C15orf22 | UNC5H3 | A-152E5.2 | hCG_27034 | HLA-G |
| TMEM108 | UNQ5357/PRO1078 | VCAM1 | VSTM2B | ICOSLG | ENG |
| KIAA1690 | THBD | L1CAM | WSCD2 | hCG_401312 | hCG_18549 |
| UNQ1875/PRO4318 | THRM | UNC5D | KIAA0789 | NTRK2 | PCDH9 |
| TNFSF12 | RELT | KIAA1777 | XYLT1 | hCG_1985371 | hCG_2026614 |
| APO3L | TNFRSF19L | UNC5H4 | XT1 | RTN1 | ICAM2 |
| DR3LG | TRPV5 | UNQ6012/PRO34692 | XYLT2 | NRP2 | hCG_41817 |
| UNQ181/PRO207 | ECAC1 | VAMP2 | XT2 | hCG_15204 | VLDLR |
| TOR1AIP1 | TRHDE | SYB2 | UNQ3058/PRO9878 | SCN2B | hCG_27927 |
| LAP1 | UNQ2507/PRO5995 | VAPA | VSIG4 | hCG_41149 | CX3CL1 |
| TNFRSF21 | TXNDC15 | VAP33 | CRIg | PRNP | hCG_15105 |
| DR6 | C5orf14 | UST | Z39IG | hCG_1785425 | ENG |
| UNQ437/PRO868 | UNQ335/PRO534 | DS2ST | UNQ317/PRO362 | SELL | TNFSF13 |

CSF mass spec transmembrane protein gene names No. 209 onwards

| 1,680-1,714 | 1,715-1,749 | 1,750-1,784 | 1,785-1,819 | 1,820-1,854 | 1,855-1,869 |
|---|---|---|---|---|---|
| hCG_2045906 | GALNT16 | hCG_39124 | SLITRK5 | SEL1L | protein |
| B4GALT1 | GALNTL1 | NGL1 | DKFZp686L0872 | GRIA4 | DKFZp686P18250 |
| SCN4B | hCG_21969 | MST065 | TM9SF3 | DKFZp779G2333 | BTCC-1 |
| hCG_1646677 | XLKD1 | hCG_2010808 | DSC3 | BDNF | PVRL1 |
| FGFR3 | hCG_23149 | SLC2A11-a | GANAB | DKFZp686A04130 | FLJ00329 |
| TLR9 | LST3 | SLC2A11 | DKFZp686D1354 | UNC5C | DKFZp686J1169 |
| GalNAc-T18 | DIPLA1 | hCG_41106 | PTPsigma | SEMA4D | DKFZp779F0871 |
| GALNT18 | EPHB4 | SMBP | DKFZp686D04248 | variant | FLJ00385 |
| GALNTL4 | hCG_20448 | hCG_25781 | HUT11 | protein | PVRL2 |
| hCG_1991780 | tcag7.1248 | ERBB4 | HMP19 | DKFZp686I11137 | DKFZp434F011 |
| HLA-G3 | RHAG | EPHB2 | DKFZp779D0769 | ABC1 | Nbla00271 |
| HLA-G | COX7A2 | variant | NBC | ADAM17 | FLJ00095 |
| CSF2RB | NEO1 | protein | SORCS3 | DST | DKFZp686B1310 |
| ADAM17 | UMOD | PCDH10 | IL28RA | DKFZp761O2023 | DKFZp686F1789 |
| ADAM18 | MRDS1 | ICAM5 | CLSTN3 | Tbeta | KIAA1149 |
| hCG_23065 | OFCC1 | NTRK2 | ANTXR1/NNG1 | RIIC | |
| ADAM8 | DKFZp761B182 | EPHA4 | fusion | FLJ00383 | |
| EGFR | POMGNT1 | DKFZp451E1911 | ATP11B | SLC4A4 | |
| PGRMC1 | SLC2A11-c | STK-1 | AAT | KIAA0921 | |
| hCG_23188 | SLC2A11 | JAG1 | CLSTN1 | DKFZp761D171 | |
| hEMMPRIN | hCG_41106 | L1CAM | PTPRN | nma | |
| BSG | PIGA | Fc-gamma | SEMA4C | RH50 | |
| hCG_20562 | hCG_1783055 | receptor | HCN1 | kit | |
| SLITRK2 | PTPRG | IIIB | TYRO3 | ATP1B1 | |
| hCG_1646164 | SLC2A11 | WUGSC: H_DJ1137M13.1 | DLK2 | KIAA0811 | |
| PRNP | hCG_41106 | CD93 | tmp_locus_20 | DKFZp686I0613 | |
| hCG_2045906 | ATP1B1 | LAR | IFNLR1 | P3.58 | |
| RAMP3 | hCG_37798 | ITGA7 | Nbla00445 | DKFZp313P2036 | |
| hCG_17148 | SRPRB | variant | CRIM1 | HCN3 | |
| tcag7.792 | hCG_2023561 | protein | CD58 | ODZ1 | |
| RHAG | P2RY14 | SCCA1 | UT-B1 | DKFZp686P14120 | |
| hCG_20861 | hCG_20914 | DKFZp564A026 | DKFZp761N1221 | SEMA6B | |
| NEU1 | FAM3A | DKFZp781F1414 | DKFZp686C2268 | CSPG3 | |
| hCG_43692 | D4S234E | LRIG1 | MGAT5 | variant | |

neuron specific genes in mouse, gene numbers 209 onwards

| 209-243 | 244-278 | 279-314 | 315-349 | 350-384 | 385-419 |
|---|---|---|---|---|---|
| CHAT | COE2 | KIAA0949 | DUSP14 | HEK3 | FBXO41 |
| CNIH3 | CRH | STK21 | MKP6 | KIAA1459 | FBX41 |
| CILP | CPLX2 | SDR39U1 | DUSP4 | EMX2 | KIAA1940 |
| UNQ602/PRO1188 | COL11A1 | C14orf124 | MKP2 | ELAVL2 | FAM183A |
| CITED2 | COLL6 | HCDI | VH2 | HUB | FAM131C |
| MRG1 | CSRNP3 | GJD2 | ECEL1 | ELAVL4 | C1orf117 |
| CAMK2N2 | FAM130A2 | GJA9 | XCE | HUD | FAM184B |
| CLDN3 | TAIP2 | GAD1 | UNQ2431/PRO4991 | PNEM | KIAA1276 |
| C7orf1 | CPLX3 | GAD | DEPTOR | EPHA7 | FAM196A |
| CPETR2 | Nbla11589 | GAD67 | DEPDC6 | EHK3 | C10orf141 |
| CNKSR2 | CPNE4 | DAPK1 | DRD5 | HEK11 | FNBP1L |
| CNK2 | CRHR2 | DAPK | DRD1B | ELMOD1 | C1orf39 |
| KIAA0902 | CRF2R | DCX | DRD1L2 | ERC2 | TOCA1 |
| KSR2 | CRH2R | DBCN | DOC2A | KIAA0378 | FAT3 |
| CNTNAP3B | CPNE6 | LISX | DPYSL5 | FAM150B | CDHF15 |
| CASPR3B | C18orf42 | DCLK3 | CRMP5 | UNQ542/PRO1097 | KIAA1989 |
| CNNM1 | CLSTN3 | DCAMKL3 | ULIP6 | ENOX1 | FIBCD1 |
| ACDP1 | CS3 | DCDC3C | EEF1A2 | PIG38 | UNQ701/PRO1346 |
| CNTN3 | KIAA0726 | KIAA1765 | EEF1AL | EOMES | FLRT3 |
| KIAA1496 | CRTAC1 | DSP | STN | TBR2 | KIAA1469 |
| PANG | ASPIC1 | DIRAS2 | EFHC2 | ESRRG | UNQ856/PRO1865 |
| CNTNAP5 | CEP68 | DLX1 | EFNB2 | ERR3 | FBXL2 |
| CASPR5 | CTXN2 | DLX5 | EPLG5 | ERRG2 | FBL2 |
| NR2F2 | SS18L1 | DGKK | HTKL | KIAA0832 | FBL3 |
| ARP1 | CREST | DLG2 | LERK5 | NR3B3 | FGF13 |
| TFCOUP2 | KIAA0693 | DLX2 | ENO2 | FAM163B | FHF2 |
| CYP4X1 | TMEM63C | DISP2 | EFNA3 | C9orf166 | FGF18 |
| UNQ1929/PRO4404 | C14orf171 | DISPB | EFL2 | FAM43B | UNQ420/PRO856 |
| CORT | CSC1 | KIAA1742 | EPLG3 | FAM78B | FGF14 |
| UNQ307/PRO350 | CXADR | DNAJC27 | LERK3 | FANK1 | FHF4 |
| CPNE5 | CAR | RABJS | ENTPD3 | HSD13 | FGF9 |
| KIAA1599 | CYB561 | RBJ | CD39L3 | UNQ6504/PRO21382 | FMN1 |
| COL6A2 | CIT | DMRTA2 | EPHA8 | FAM155A | FMN |
| EBF2 | CRIK | DMRT5 | EEK | FBLL1 | LD |

neuron specific genes in mouse, gene numbers 209 onwards

| 420-454 | 455-489 | 490-524 | 525-559 | 560-594 | 595-629 |
|---|---|---|---|---|---|
| FIBIN | GHSR | GLUR1 | HCN3 | EWI3 | IRX2 |
| PSEC0235 | GABRA5 | SLC2A14 | KIAA1535 | KIAA0466 | IRXA2 |
| FSTL4 | GDNF | GLUT14 | HOOK1 | ISOC1 | MAPK8IP2 |
| KIAA1061 | GUCY1A2 | GLUT3 | HS3ST5 | CGI-111 | IB2 |
| FBXL16 | GUC1A2 | GPR135 | 3OST5 | IL34 | JIP2 |
| C16orf22 | GUCSA2 | GRIP1 | HS3OST5 | C16orf77 | PRKM8IPL |
| FBL16 | GFRA4 | GRM1 | HEXIM2 | INHA | KIAA1549L |
| FRMPD3 | GLRA2 | GPRC1A | L3 | INSL5 | C11orf41 |
| KIAA1817 | MSTN | MGLUR1 | HAS3 | UNQ156/PRO182 | C11orf69 |
| FSTL5 | GDF8 | GRPR | IGF2BP2 | INSM2 | KCTD16 |
| KIAA1263 | GIPC2 | GDA | IMP2 | IA6 | KIAA1317 |
| FUT7 | GLB1L2 | KIAA1258 | VICKZ2 | Nbla106 | KCNJ5 |
| GALR2 | MSTP014 | GPR26 | HPCA | IRS4 | GIRK4 |
| GALNR2 | UNQ210/PRO236 | GPR21 | BDR2 | ISLR | KCNF1 |
| GPRASP2 | GPR149 | GPR61 | IGFBPL1 | UNQ189/PRO215 | KCNJ3 |
| GNG3 | PGR10 | BALGR | IGFBPRP4 | PKIA | GIRK1 |
| GNGT3 | GNAZ | HS6ST2 | ICA1L | PRKACN1 | CAMK2B |
| FOXO6 | GNL3L | PSEC0092 | ALS2CR14 | IQSEC3 | CAM2 |
| GNG2 | GNRH1 | GPR27 | ALS2CR15 | KIAA1110 | CAMK2 |
| GABRB3 | GNRH | SREB1 | IGF1 | JAKMIP1 | CAMKB |
| FRMD3 | GRH | GRID2IP | IBP1 | GABABRBP | KCNA3 |
| EPB41L4O | LHRH | GPR22 | IL17B | JAMIP1 | HGK5 |
| GARNL3 | GPR151 | GPRIN1 | IL20 | MARLIN1 | KCNA4 |
| GDF5 | PGR7 | KIAA1893 | NIRF | PRKAR1B | KCNA4L |
| BMP14 | GLOD5 | GPRIN3 | ZCYTO7 | INSM1 | KCNB2 |
| CDMP1 | GLRA3 | KIAA2027 | UNQ516/PRO1031 | IA1 | KCNC1 |
| GALR1 | GPR158 | GREM2 | PKIB | KCNC2 | KIT |
| GALNR | KIAA1136 | CKTSF1B2 | PRKACN2 | KCNH1 | SCFR |
| GALNR1 | GPR45 | DAND3 | IKZF4 | EAG | KIAA0319 |
| GPRASP1 | GPR88 | PRDC | KIAA1782 | EAG1 | KIAA0895L |
| GASP | STRG | HAP1 | ZNFN1A4 | KCNK4 | PRKAR2B |
| KIAA0443 | GPR12 | HAP2 | INSRR | TRAAK | KCNIP4 |
| GATSL2 | GRIA1 | HLP1 | IRR | KCNS2 | CALP |
| GATSL1 | GLUH1 | HCN4 | IGSF3 | KIAA1144 | KCHIP4 |

neuron specific genes in mouse, gene numbers 209 onwards

| 630-664 | 665-699 | 700-734 | 735-769 | 770-804 | 805-839 |
|---|---|---|---|---|---|
| KCNG3 | UNQ9234/PRO31993 | CCDC109B | JNK1 | NDRF | NPTX1 |
| KCNH4 | PPFIA2 | MCUB | PRKM8 | AVP | NPTXR |
| KCNK9 | DLEU7 | MAP3K15 | SAPK1 | ARVP | NRSN2 |
| TASK3 | LEU7 | ASK3 | SAPK1C | VP | C20orf98 |
| KLF5 | LHX8 | MAGEE1 | MLF1 | CHL1 | NTSR1 |
| BTEB2 | L3MBTL1 | HCA1 | MPPED1 | CALL | NTRR |
| CKLF | KIAA0681 | KIAA1587 | C22orf1 | GAP43 | NMBR |
| IKLF | L3MBT | MARCH9 | FAM1A | NEUROD1 | GRIN2D |
| KIAA2022 | L3MBTL | RNF179 | MRAP2 | BHLHA3 | GluN2D |
| CAMK1G | ALOXE3 | MARCH4 | C6orf117 | NEUROD | NMDAR2D |
| CLICK3 | LSM11 | KIAA1399 | MTUS2 | NTS | GRIN1 |
| VWS1 | LGI1 | RNF174 | CAZIP | NOV | NMDAR1 |
| KLHL34 | EPT | MESP2 | KIAA0774 | CCN3 | NPAS4 |
| KCNIP2 | UNQ775/PRO1569 | BHLHC6 | TIP150 | IGFBP9 | BHLHE79 |
| KCHIP2 | PPFIA4 | SCDO2 | RUNX1T1 | NOVH | NXF |
| KIFC2 | KIAA0897 | MET | AML1T1 | NDST3 | PASD10 |
| KLHDC8A | LRRC3B | MAB21L1 | CBFA2T1 | HSST3 | NPFFR1 |
| LANCL3 | LRP15 | CAGR1 | COR | UNQ2544/PRO4998 | GPR147 |
| LANCL2 | UNQ195/PRO221 | Nbla00126 | ETO | NDST4 | NPFF1 |
| GPR69B | L3MBTL4 | MAB21L3 | MTG8 | HSST4 | NPY2R |
| TASP | LMX1A | C1orf161 | ZMYND2 | NNMT | NRN1 |
| LHFPL4 | LONRF2 | MCTP2 | MSI2 | NECAB1 | NRN |
| LHX5 | RNF192 | MEX3A | MYH8 | EFCBP1 | NTNG2 |
| L1CAM | LRRC16B | RKHD4 | NANOS2 | NDN | KIAA1857 |
| CAML1 | C14orf121 | MESP1 | NOS2 | NEFH | LMNT2 |
| MIC5 | LRRN4CL | BHLHC5 | SLC24A2 | KIAA0845 | UNQ9381/PRO34206 |
| LIN28B | UNQ728/PRO1410 | MGAT4C | NCKX2 | NFH | NUMBL |
| CSDD2 | LRRC26 | MMP24 | NDRG4 | TACR1 | NAP1L2 |
| LHFPL1 | CAPC | MT5MMP | BDM1 | NK1R | BPX |
| UNQ5824/PRO19643 | MAGEE2 | MICAL2 | KIAA1180 | TAC1R | NUDT11 |
| LHX9 | HCA3 | KIAA0750 | SLC24A3 | NPY | APS1 |
| LINGO2 | MAGEL2 | MICAL2PV1 | NCKX3 | OLFM3 | DIPP3B |
| LERN3 | NDNL1 | MICAL2PV2 | NEUROD2 | NOE3 | SLC10A4 |
| LRRN6C | LY6H | MAPK8 | BHLHA1 | UNQ1924/PRO4399 | NXPH2 |

neuron specific genes in mouse, gene numbers 209 onwards

| 840-874 | 875-909 | 910-944 | 945-979 | 980-1,014 | 1,015-1,049 |
|---|---|---|---|---|---|
| NPH2 | PDCD1LG2 | PGF | BRN3A | RGS78P | KIAA1391 |
| OPCML | B7DC | PGFL | RDC1 | R7BP | RGS17 |
| IGLON1 | CD273 | PLGF | PNMAL2 | RAB6B | RGMB |
| OBCAM | PDCD1L2 | PLXNA4 | KIAA1183 | RASSF6 | RHBDL1 |
| OPN3 | PDL2 | KIAA1550 | PRRC2B | RBMS3 | RHBDL |
| ECPN | PDE1A | PLXNA4A | BAT2L | RAB3B | ARHGAP36 |
| OPRL1 | PCDHA1 | PLXNA4B | BAT2L1 | RAB3C | RIMS3 |
| OOR | PCDHA8 | UNQ2820/PRO34003 | KIAA0515 | CRABP2 | KIAA0237 |
| ORL1 | PCGF2 | PLD5 | PRRT3 | RAI2 | RIMBP2 |
| PAFAH1B3 | MEL18 | PIP5KL1 | UNQ5823/PRO19642 | RAMP3 | KIAA0318 |
| PAFAHG | RNF110 | PNMA2 | PRDM8 | RARB | RBP2 |
| ADCYAP1 | ZNF144 | KIAA0883 | PFM5 | HAP | RIMS4 |
| PACSIN1 | PELI3 | MA2 | PPP1R1B | NR1B2 | C20orf190 |
| KIAA1379 | PGBD5 | PNOC | DARPP32 | REM2 | RIPK4 |
| OVGP1 | PGM2L1 | OFQ | PSD | RBM24 | ANKRD3 |
| MUC9 | BM32A | PROKR2 | EFA6 | RNPC6 | DIK |
| OGP | PCDHA9 | GPR73L1 | KIAA2011 | RGAG1 | RNF39 |
| PCDHAC2 | KIAA0345 | PKR2 | PSD1 | KIAA1318 | HZFW |
| PCLO | CD274 | PLCH2 | TYL | RHOV | RNF165 |
| ACZ | B7H1 | KIAA0450 | PRLHR | ARHV | RNASEL |
| KIAA0559 | PDCD1L1 | PLCL4 | GPR10 | WRCH2 | RNS4 |
| P2RY1 | PDCD1LG1 | PNMA3 | GR3 | RESP18 | RPS6KL1 |
| PCDHA2 | PDL1 | MA3 | PRRT4 | RALGPS2 | RPP25 |
| PCDHA10 | PODXL2 | PPP3CB | PTCHD1 | RGS11 | RSPO3 |
| CNRS8 | UNQ1861/PRO3742 | CALNA2 | RAB9B | ADARB1 | PWTSR |
| TP73 | PGRMC1 | CALNB | RAB9L | ADAR2 | THSD2 |
| P73 | HPR6.6 | CNA2 | QRFPR | DRADA2 | RTBDN |
| PLA2G4E | PGRMC | PPM1E | GPR103 | RED1 | RTL1 |
| PAQR9 | PITPNM2 | CAMKN | RAB3D | RELL2 | MAR1 |
| PCDHA13 | KIAA1457 | KIAA1072 | GOV | C5orf16 | MART1 |
| CNRS5 | NIR3 | POPX1 | RAB16 | UNQ9423/PRO34565 | PEG11 |
| PCSK5 | PIWIL2 | PNMA1 | RTN4RL2 | REPS2 | RSPH4A |
| PC5 | HILI | MA1 | NGRH1 | POB1 | RSHL3 |
| PC6 | PLCXD3 | POU4F1 | NGRL3 | ARHGAP20 | RSPO1 |

neuron specific genes in mouse, gene numbers 209 onwards

| 1,050-1,084 | 1,085-1,119 | 1,120-1,154 | 1,155-1,189 | 1,190-1,224 | 1,225-1,259 |
|---|---|---|---|---|---|
| SLC27A3 | FRCL1 | SIX3 | SUSD2 | TM4SF5 | THH |
| ACSVL3 | SLC5A7 | SLCO5A1 | STX1B | TUBB2B | THL |
| FATP3 | CHT1 | OATP5A1 | STX1B1 | TCEAL7 | TRHY |
| PSEC0067 | SCML4 | SLC21A15 | STX1B2 | THSD7B | TRIM46 |
| UNQ367/PRO703 | CXCL12 | SPRN | STX19 | KIAA1679 | TRIFIC |
| SLC45A1 | SDF1 | SHO | SYTL1 | SPOCK3 | TRIM66 |
| DNB5 | SDF1A | SNTG2 | SLP1 | TICN3 | C11orf29 |
| SLC6A17 | SDF1B | SRRM4 | SB146 | UNQ409/PRO771 | KIAA0298 |
| NTT4 | SCN2B | KIAA1853 | SNCA | TUBG2 | NGFR |
| RUNDC3B | UNQ326/PRO386 | SPHKAP | NACP | TBR1 | TNFRSF16 |
| RPIB9 | SCRT1 | KIAA1678 | PARK1 | TCP11 | TRIM67 |
| RPIP9 | SBSN | SKIP | TMEM200A | TDRD6 | TNL |
| SLC22A15 | UNQ698/PRO1343 | SULT2B1 | KIAA1913 | TAC3 | TRPC6 |
| FLIPT1 | SEPT6 | HSST2 | HBE61 | NKNB | TRP6 |
| UNQ9429/PRO34686 | KIAA0128 | STMN1 | SYN1 | UNQ585/PRO1155 | TRPC5 |
| SLC35F4 | SEP2 | C1orf215 | MAPT | TMEM59L | TRP5 |
| C14orf36 | SHANK2 | LAP18 | MAPTL | BSMAP | TSPAN11 |
| SATB2 | CORTBP1 | OP18 | MTBT1 | C19orf4 | TH |
| KIAA1034 | KIAA1022 | SERTAD4 | TAU | TMTC4 | TYH |
| SCN2A | PROSAP1 | STK32B | TUBB2A | TEX15 | UBE2QL1 |
| NAC2 | SH3GL2 | YANK2 | TUBB2 | TIAM2 | USP11 |
| SCN2A1 | CNSA2 | UNQ3003/PRO9744 | TCERG1L | KIAA2016 | UHX1 |
| SCN2A2 | SH3D2A | SULT4A1 | SYT2 | STEF | TTC9B |
| SCUBE3 | SEPT3 | SULTX3 | TCEAL5 | TNRC6C | SLC25A27 |
| CEGF3 | SEP3 | STMN3 | TERT | KIAA1582 | UCP4 |
| SCRT2 | SH2D4B | SCLIP | EST2 | TNFAIP8L3 | UNQ772/PRO1566 |
| FP7030 | SST | SOX11 | TCS1 | TIPE3 | UPK3A |
| SLC7A14 | SP9 | SPDEF | TRT | TMEM158 | UPK3 |
| KIAA1613 | SH3BGRL2 | PDEF | SYT3 | HBBP | TSPYL4 |
| SCN3B | FASH3 | PSE | SYT6 | RIS1 | KIAA0721 |
| KIAA1158 | SH3RF3 | SSTR4 | TMEM178A | TMEM169 | TTC9 |
| SCN9A | POSH2 | SV2B | TMEM178 | LHFPL5 | KIAA0227 |
| NENA | SH3MD4 | KIAA0735 | PSEC0131 | TMHS | TTC9A |
| SLC35D3 | SIDT1 | SSTR2 | UNQ5926/PRO19820 | TCHH | USP27X |

neuron specific genes in mouse, gene numbers 209 onwards

| 1,260-1,294 | 1,295-1,329 | 1,330-1,364 | 1,365-1,399 | 1,400-1,434 | 1,435-1,469 |
|---|---|---|---|---|---|
| USP22L | VGLUT2 | RBFOX2 | SPRYD3 | TUFT1 | LMTK3 |
| USP27 | SLC17A8 | SGIP1 | ZNF483 | UNQ865 | BCL11A |
| USP29 | VGLUT3 | MYO5B | TRPC7 | OPRD1 | MAL2 |
| UBQLN2 | VWC2 | CKMT1B | LRRC24 | MAPK10 | KLHL14 |
| N48P4 | UNQ739/PRO1434 | CKMT1A | hCG_1818221 | PDZD4 | SYT17 |
| PLIC2 | WSCD2 | SYNGR3 | WDR6 | hCG_2004980 | OSCAR |
| HRIHFB2157 | KIAA0789 | NELL1 | LMCD1 | ZDBF2 | SDSL |
| UFSP1 | XKR7 | ALLC | PLEKHA5 | DACT1 | KLHL1 |
| UPP2 | C20orf159 | hCG_15830 | ZMAT4 | NBEA | RLTPR |
| ATP6AP1L | XRG7 | RTN1 | INPP5J | NAPB | CX3CL1 |
| VAX1 | YDJC | CHRFAM7A | ADCY1 | hCG_22369 | IPCEF1 |
| SLC32A1 | ZFP57 | PRTN3 | hCG_19245 | SEMA4G | RASGRF2 |
| VGAT | C6orf40 | EPHB6 | GRIN2B | AUTS2 | KCNT1 |
| VIAAT | ZNF698 | SLC38A1 | SMARCA1 | NOS1 | CD24 |
| VWA5A | ZNF558 | ISLR2 | hCG_1980650 | PCDHA4 | HOMER2 |
| BCSC1 | ZNF575 | P2RX2 | KCNT2 | RSPO2 | PCSK1 |
| LOH11CR2A | ZWINT | RASGEF1C | NRG3 | FAM171A2 | CACNA1A |
| VSNL1 | ZCCHC18 | EYA2 | NFIX | CYGB | SYT7 |
| VISL1 | ZFR2 | CADPS | MMP11 | SV2C | KCNH7 |
| SLC18A2 | KIAA1086 | DOC2B | HDGFRP3 | CCDC73 | SLIT1 |
| SVMT | ZIC1 | hCG_1741343 | ATP6V1G2-DDX39B | NTRK1 | GPR83 |
| VMAT2 | ZIC | OCC-1 | CALB2 | CELF5 | PLEK2 |
| VSTM2L | ZNF201 | C12orf75 | hCG_2011413 | RBMS1 | CACNB2 |
| C20orf102 | ZIC4 | SERF1B | SP8 | RIT2 | PLCD3 |
| VWC2L | ZNF711 | SERF1A | hCG_38927 | FABP3 | RXFP1 |
| SLC18A3 | CMPX1 | FAM159B | TRPC4 | DUSP26 | LGI2 |
| VACHT | ZNF6 | DIABLO | SCN5A | CHODL | PLEKHA7 |
| VASH2 | TUBB3 | hCG_1782202 | CYP4B1 | CDK5R1 | CCNF |
| VASHL | hCG_1983504 | EPHB2 | hCG_22100 | LRFN5 | SYT1 |
| XKR4 | AHI1 | SLC27A2 | ARHE | RBFOX1 | KCNIP1 |
| KIAA1889 | PNCK | hCG_39815 | RND3 | RALGAPA2 | CACNA1B |
| XRG4 | DLGAP2 | SNAP91 | SNCB | FKBP1B | BCL11B |
| SLC17A6 | VGF | SYP | CHCHD10 | CELF3 | HS3ST6 |
| DNPI | FOXP2 | KCNH2 | PCBP3 | RGS4 | GRM2 |

neuron specific genes in mouse, gene numbers 209 onwards

| 1,470-1,504 | 1,505-1,539 | 1,540-1574 | 1,575-1,609 | 1,610-1,644 | 1,645-1,679 |
|---|---|---|---|---|---|
| STMN2 | VWA5B2 | ZNF385B | PLA2G4F | COL19A1 | MTSS1 |
| NEGR1 | NXPH3 | OSBPL5 | SPATA2L | SNAP25 | PDZD2 |
| TRPC3 | WNT7B | FOSL2 | ATP1B1 | CNTNAP2 | WIF1 |
| GALNTL6 | KSR2 | SLC6A7 | CLCN1 | PPARGC1A | MAML3 |
| GCA | DBN1 | LYPD6B | MCHR1 | GSE1 | UNC5D |
| PIK3C2G | VIP | DKK3 | SAMD14 | INA | OLFM1 |
| EPHA3 | SGSM1 | FLYWCH2 | KCNQ5 | GCGR | TEKT2 |
| SH2D5 | SNX16 | MYT1L | ADARB2 | EFNA5 | GRIN2A |
| ANKRD55 | DBNDD1 | CLSTN2 | FAM65B | DMRT3 | PALM2 |
| PPARGC1B | CXXC4 | CYP2S1 | nan | ACTL6B | SLC16A14 |
| SYT4 | FSIP1 | RAB3A | CRABP1 | POU6F2 | VPS37D |
| SYT13 | CACNA1C | ELN | CNTN5 | GABRG3 | FAM57B |
| FRMPD4 | SYT10 | FAM167A | PLCZ1 | SEZ6L2 | C8orf89 |
| SLC2A13 | COL25A1 | PENK | MAP3K12 | KCNQ2 | PALM3 |
| GLIS1 | GNAL | ACR | YPEL4 | SPAG6 | NETO1 |
| KLHL29 | DYNC1I1 | GABRG2 | MME | CLEC2L | NANOS1 |
| SYT5 | RAB11FIP4 | ANGPT1 | TMEM210 | LAMA3 | NKX2-1 |
| SYNPR | COL24A1 | COL23A1 | TRIM17 | CBLN2 | RAC3 |
| RGS8 | ZIC2 | KCNJ11 | OSBP2 | CHGB | PLCXD2 |
| CASP3 | KIAA1211 | NXNL2 | UNC13A | EML5 | SEZ6 |
| IL12A | SHISA6 | MN1 | CA11 | SLC38A4 | SELV |
| RCAN2 | TMEM145 | RELN | SLC7A4 | MCTP1 | SEMA6C |
| NHLH2 | RTN4R | RFX2 | WNT2 | EBF3 | ADD2 |
| ZPBP | COCH | TBC1D30 | NRG1 | RNPC3 | WNT5A |
| B3GALNT1 | TRPV1 | FAM189A1 | GALNT14 | DSCAML1 | NFIB |
| ANK1 | CPLX1 | RUSC1 | CPNE7 | HECW1 | DNM1 |
| TRHDE | SERPINI1 | GIPR | CRMP1 | DKKL1 | ANKRD34A |
| ANKRD42 | TTC39A | HTR1A | SERP2 | C3orf18 | B4GALNT4 |
| SSBP2 | TMEM91 | ARMCX4 | TAS1R1 | CDKL4 | nan |
| SRRM3 | SLC1A6 | DCC | SGTB | PITPNM3 | STXBP5L |
| SHANK1 | GPC1 | PRKCE | POPDC3 | RADIL | MAP3K9 |
| PEG3 | DGKG | LGALS7 | NEUROD6 | NR1H4 | GBX2 |
| MTMR7 | MARCH11 | SMPD3 | KIAA1107 | TRPV6 | LHX1 |
| CADM3 | RNF152 | ARHGDIG | CNR1 | C14orf39 | ARHGAP44 |

neuron specific genes in mouse, gene numbers 209 onwards

| 1,680-1,714 | 1,715-1,749 | 1,750-1,784 | 1,785-1,819 | 1820-1854 | 1855-1881 |
|---|---|---|---|---|---|
| PAK3 | CGN | DLX6 | CNRIP1 | MYL6B | GDAP1 |
| PCP4 | SLC4A3 | HOOK2 | NOL4 | SLC12A5 | TRANK1 |
| SLC6A15 | AGBL4 | HLA-B | CHRNA3 | TMEM150C | C5orf49 |
| CNTNAP4 | GDAP1L1 | NXPH4 | PAK6 | NAP1L3 | C1QTNF4 |
| CDKL2 | GRM8 | LMO3 | HSPA12A | UCHL1 | SYN3 |
| NPY1R | STXBP1 | RALYL | ADCY8 | SVOP | COL6A1 |
| SNTB1 | PANX2 | COX7A1 | ST8SIA2 | UBE2O | DACH1 |
| NOVA2 | TRO | DIRAS1 | PRPH | MDGA1 | ZNF667 |
| KIF5C | LRP11 | JPH4 | CDH9 | THSD7A | LHX6 |
| KCNAB1 | GRM7 | PLEKHG4 | PKNOX2 | SYN2 | UNC119 |
| ACTA1 | PCNXL2 | ROBO2 | SPOCK1 | FAM49A | IGSF9 |
| OXTR | CRHBP | CLRN1 | CAMTA1 | FAM169A | TWSG1 |
| PRKAA2 | KCTD8 | SCG2 | SARM1 | ABCA5 | ASS1 |
| VSTM2A | TMEM130 | VWCE | ZNF804B | CLVS1 | CAMK4 |
| CALN1 | KCNG1 | NTN5 | RUFY3 | RASGRP2 | CNIH2 |
| SLC25A22 | LRRC7 | ITGA3 | SMS | ZBTB16 | SRGAP3 |
| GALNT9 | ANKRD34B | PTH2R | SDK2 | PRUNE2 | NRIP3 |
| CD200 | CIDEA | FXYD7 | HRH3 | CCDC64 | CSDC2 |
| ATP1A3 | HS6ST3 | GOLGA7B | TMEM198 | HMGCLL1 | CCDC136 |
| CA10 | CCBE1 | ANKRD35 | ARHGAP15 | NIPSNAP1 | BEND4 |
| KIAA0895 | AK5 | STAC | HFM1 | MST1R | B4GALT6 |
| NPAS1 | CDKL1 | MAST1 | CCDC120 | SRSF12 | UNC5A |
| PTPRN | ZDHHC22 | CBX7 | INPP5F | YBX2 | MKRN3 |
| NRXN3 | CHD5 | C10orf35 | CDH8 | PGR | RASGEF1A |
| ARMCX1 | SPIRE2 | SAMD3 | C1orf95 | SFRP4 | CDHR1 |
| RAPGEFL1 | C2orf80 | PTPRN2 | SOBP | IL1RAPL2 | THY1 |
| SEMA3A | ABLIM3 | CSRNP2 | GABRA2 | EPHA6 | |
| ATL1 | JAZF1 | PPP2R2C | DPYSL3 | ISL1 | |
| TTC22 | KRT73 | CIB2 | YPEL1 | FNDC3B | |
| THPO | SAMD10 | ADPRHL1 | GAD2 | PNMAL1 | |
| C3orf67 | GRIP2 | ZNF804A | CDON | HRK | |
| TMEM30C | RAB26 | STK32C | NPNT | ADAMTS17 | |
| KIAA1324 | PPIF | AMN | MGAT5B | ERO1B | |
| ITGBL1 | KRT79 | RHOF | SPINT2 | LEFTY1 | |

neuron specific genes in mouse, gene numbers 209 onwards

| 209-243 | 244-278 | 279-314 | 315-349 | 350-384 | 385-419 |
|---|---|---|---|---|---|
| IGF1 | CDH12 | TDO2 | SLC7A3 | KCTD16 | PAK6 |
| IGSF3 | AP1S2 | NYAP2 | SH2D5 | GPR22 | PCDH18 |
| LYPD6B | LOC100128264 | LOC728739 | NEK10 | PLK2 | BCL11B |
| KIAA1324 | DACH2 | CLUL1 | TRNP1 | FLRT3 | RAB27B |
| KIAA1217 | SHISA9 | TUSC3 | TSPAN13 | SCN3A | ATP6V1G2 |
| KCNC1 | AFF2 | SCG5 | DNM1 | FBLN7 | PAK7 |
| XK | BCL11A | RFPL1-AS1 | ITGBL1 | PTPN5 | CDK5R1 |
| GNG3 | HAPLN1 | ATP1B1 | NT5DC3 | MAGED4 | PABPC1L2B |
| FAM190A | TAGLN3 | MEPE | CREG2 | MAGED4 | ADAMTS6 |
| SP8 | NETO2 | PLXNA4 | ST8SIA5 | RFPL2 | NPTX2 |
| D4S234E | TMEM130 | BSCL2 | NRG1 | LOC284408 | LPHN2 |
| NAPB | NOS1AP | CLSTN2 | CADPS | HBG1 | NSFP1 |
| NEUROD6 | COL12A1 | ACCN1 | FAM183A | TRPC6 | VWC2 |
| CACNG3 | TTC9 | RSPO2 | PCDH11X | C11orf41 | IGFBP5 |
| FAXC | PPM1E | CDH8 | TOX2 | ANKRD34A | ENC1 |
| PKP2 | CNTNAP2 | GRM5 | KIAA1024 | KIAA1456 | ZNF204P |
| PPP1R1C | PRMT8 | ATP2B3 | SEZ6 | OSBPL10 | CD200 |
| CNNM1 | DYNC1I1 | STXBP5L | LHX6 | AMPH | TOX3 |
| NDST4 | CNTN4 | CHRM3 | LINC00314 | B4GALT6 | CPLX3 |
| TRHDE | BEND4 | PAR1 | PCLO | GPR149 | SLC27A2 |
| EYA4 | ELAVL4 | FGF9 | DLX2 | LINC00473 | ANK1 |
| SCN8A | SYCE1 | LPPR4 | PABPC1L2A | SYN1 | CELF4 |
| KCNS2 | CNTN3 | GNG2 | SULT4A1 | RCAN2 | PLD5 |
| TRPC5 | PRKCE | SH3GL2 | THSD7B | RNF207 | UBE2T |
| PTH2R | MIMT1 | SCN3B | RYR2 | LPPR5 | NXPH1 |
| SNCB | RBM11 | C2orf80 | LOC440894 | GNG4 | BEX1 |
| SV2A | HTR2A | INPP5F | NAP1L2 | SORCS3 | ANKRD55 |
| TMEM196 | TCEAL6 | ELMOD1 | ADD2 | NAP1L3 | KCNB1 |
| EFNB3 | FGF12 | CAP2 | HTR1E | ZNF385B | KHDRBS2 |
| SSTR2 | NSFP1 | KITLG | RPS6KA6 | HMP19 | GJB7 |
| KCNT2 | LOC440894 | C1orf173 | KCNH1 | PNMA2 | FAM216A |
| NOV | GDA | MLLT11 | HS6ST3 | SLC26A8 | SORCS1 |
| LOC653653 | SYT16 | NUDT11 | KIAA1211 | IL13RA2 | TBR1 |
| CPNE4 | LOC286002 | CSRNP3 | LOC730811 | L1CAM | CCKBR |

neuron specific genes in mouse, gene numbers 209 onwards

| 420-454 | 455-489 | 490-524 | 525-559 | 560-594 | 595-629 |
|---|---|---|---|---|---|
| SLC6A15 | RNF165 | SNTG1 | KCNH5 | SULF1 | MAGEL2 |
| SYP | LINC00246A | NRSN1 | OPCML | FBXL2 | ADAM22 |
| PRKAR2B | ARL9 | RGMB | NEFL | DIRAS3 | MEX3B |
| DRP2 | GLRA3 | SLC6A17 | PROK2 | CABP1 | THSD7A |
| LOC100507043 | VAT1L | LRRC7 | KLHL13 | FXYD6 | SLC26A4 |
| GALNT14 | SV2B | GYPE | RGS17 | CCDC152 | PSMG4 |
| PEG3 | SNAP91 | CLVS2 | PLCB4 | FAM123C | THRB |
| KLF5 | PAR5 | SGSM1 | HPCAL4 | HMGCLL1 | GABRE |
| AKR1C2 | CACNG2 | CNTNAP5 | LRFN5 | FREM1 | MOB4 |
| CIT | PIP5K1B | FRY | PTPN3 | CELF5 | ANKRD7 |
| GRM7 | GPR137C | LOC158696 | LOC100130155 | CHRNA2 | CSMD1 |
| PDE1B | STAR | ANKRD30BL | DLGAP2 | BEX2 | PAM |
| NELL1 | GABRB3 | FAM81A | ADRBK2 | PLXNC1 | WIPF3 |
| CHRNA7 | DOK6 | KCNA4 | KCNA6 | THY1 | MICAL2 |
| KCNIP4 | NCALD | PCP4 | DLGAP1 | CACNA2D3 | ANKRD34C |
| GRIA4 | CACNA1B | SETD6 | ACRV1 | RGS6 | KIAA0408 |
| LMO7 | C11orf63 | CTXN2 | CORT | LOC338651 | FGF14-IT1 |
| XKR4 | KMO | NMU | GPRASP1 | LOC440040 | FBXL21 |
| SNX10 | EPB41L4B | DCX | LRRTM2 | SGK494 | HSFY2 |
| LOC728730 | C12orf68 | ENO2 | RNF175 | LOC100506123 | C3orf80 |
| LOC389023 | LOC285954 | PDXP | IL12RB2 | EXO1 | SLITRK5 |
| RPP25 | ELOVL4 | LANCL3 | PAK3 | HPRT1 | CDK14 |
| PTGFR | RAB3A | SUSD4 | RASGRF2 | TMEM178 | HSFY2 |
| CDKL2 | SLC38A1 | NDST3 | FABP4 | SHANK2 | ROPN1L |
| C8orf85 | CSMD3 | DGKE | NUDT10 | C17orf102 | LRRC8B |
| DPP6 | RIMS3 | DIRAS1 | LOC100505483 | ARHGEF3 | TRPC7 |
| B3GALT2 | GRIA3 | TSPAN2 | DNAH14 | ZNF519 | FAAH2 |
| LHFPL5 | ARHGAP20 | IFNA21 | EPHA10 | CACNG8 | GLT1D1 |
| ADRA2A | PLS1 | UNC80 | NTF3 | JPH1 | ABCA5 |
| BASP1 | HS3ST2 | ZNF540 | GRM1 | AKAP5 | KIAA1107 |
| KCNMB2 | HS6ST2 | PNMAL1 | IBSP | MAGEE1 | GOT1 |
| SLC47A1 | C8orf4 | MAGEE2 | SLC17A8 | SYN3 | RBFOX2 |
| NAP1L5 | ODZ1 | NEGR1 | YWHAH | PDP1 | SLIT2 |
| ZFR2 | OSTN | SLITRK3 | RET | DRD1 | TCEAL5 |

neuron specific genes in mouse, gene numbers 209 onwards

| 630-664 | 665-699 | 700-734 | 735-769 | 770-804 | 805-839 |
|---|---|---|---|---|---|
| ACYP1 | GPR63 | TRIM36 | ABI3BP | LOC283683 | FRMPD2P1 |
| MIF | SLC35F3 | PREPL | DACT1 | TBC1D30 | PLEKHA8P1 |
| GDAP1L1 | RGS7 | CDCA7 | FNDC9 | ASNS | GDAP1 |
| GLS | TRIM7 | CNTNAP1 | SLC8A2 | PROX1 | SLC8A3 |
| AP3B2 | LOC100131208 | C1orf53 | CRABP1 | SCARNA13 | UNC5D |
| NR2F2 | TRIM55 | LDB2 | CNTN6 | VAMP1 | LOC441666 |
| GULP1 | ATP1A3 | GRM8 | C9orf11 | CXXC4 | FBXW12 |
| KLHL29 | OR7E2P | RPH3A | GABRA3 | TCEAL2 | LDOC1 |
| MCF2 | NPTX1 | RIMKLA | QPCT | PRKCB | RIIAD1 |
| KIAA1644 | NBEA | APITD1-CORT | GFOD1 | C11orf80 | PPEF1 |
| KIAA1524 | ST8SIA2 | ZNF483 | NPTN | CAMK2N2 | ATP6V1G2 |
| WASF1 | SLC16A7 | SPINT2 | AKAP14 | KIAA0895 | ATP6V1G2 |
| SNCA | LURAP1L | KSR2 | CELF3 | CHGA | CNIH3 |
| CERS6 | LHFPL4 | ANKRD19P | CALN1 | DUSP26 | FRAS1 |
| DAB1 | VAX1 | ST6GALNAC5 | WTH3DI | LOC100506124 | SLC9B2 |
| GPRASP2 | HCN3 | FAM216B | DMRTC1B | FAM106CP | ANKRD6 |
| GUCY1B3 | SLC17A6 | WDR69 | DMRTC1B | CHRFAM7A | GLT8D2 |
| SLC10A5 | TAS2R50 | TCTEX1D1 | RALYL | TPBG | MAGI1 |
| GPR151 | KCNV1 | GUCY1A2 | HIST1H4H | ARX | PATE4 |
| OCIAD2 | GRB14 | ADCY1 | DNAJC12 | PKD2L1 | TRAPPC2L |
| GLYATL1 | NOL4 | YWHAG | CLGN | ARHGAP28 | TSPYL1 |
| GPRIN1 | TMEM155 | GNB5 | ZNF804B | EIF4E1B | DOC2A |
| FAM65B | SLC44A5 | GCOM1 | ARNTL2 | SCAMP5 | MTMR7 |
| TUBA8 | MAP1LC3C | DLG3 | GFRA2 | SEZ6L | SUSD1 |
| C10orf35 | CCDC136 | FAM174B | DKK2 | FAM135B | BEAN1 |
| SAMD12 | ARPP21 | ARHGAP36 | RFK | PITPNM3 | HDAC9 |
| LOC100507341 | CACNA2D1 | CORO6 | PCSK1 | SLC35F2 | SLC16A14 |
| SPATA17 | TAS2R10 | SERPINI1 | RGS12 | REPS2 | GABRA4 |
| KIAA1239 | FBXO16 | COL22A1 | CELSR3 | MC4R | PRICKLE1 |
| ANKRD30B | TSHZ2 | PLCXD2 | MCHR2 | GUCA1A | MCTP1 |
| CES4A | STXBP1 | DPT | FAR2 | MOAP1 | KLC1 |
| PPFIA4 | LNX1 | FAM156B | GLRB | RASGEF1A | KCNB2 |
| JAKMIP1 | LRRC2 | HAR1B | ZNF815 | HSPA12A | PCDH19 |
| ATRNL1 | LOC284215 | FAM84A | ADARB2 | NTN4 | CAMKK1 |

neuron specific genes in mouse, gene numbers 209 onwards

| 840-874 | 875-909 | 910-944 | 945-979 | 980-1,014 | 1,015-1,049 |
|---|---|---|---|---|---|
| LRRC55 | TCEAL7 | PART1 | MAP1B | PAK1 | TAS2R16 |
| SNAI2 | MME | MDH1 | BMP3 | LRRTM3 | EPHA3 |
| HAPLN4 | CDKN3 | CACNA1C | CBLN1 | HTR7 | PAR-SN |
| RND3 | KCNIP4-IT1 | GUCY1A3 | CNKSR2 | CHST2 | ECM1 |
| LEFTY1 | LOC100506071 | C9orf125 | STS | SOX1 | CCDC165 |
| SPRED3 | REEP5 | ATP6V1G2 | OXR1 | C1orf115 | FAM43A |
| SYNDIG1L | CLSTN3 | CCNB1 | CRYM | PKIB | PTPRN2 |
| CACNB4 | SLC1A1 | GAP43 | RUSC1 | PTPRO | RUNDC3B |
| ACTC1 | LOC285484 | CHRNB2 | BHLHE22 | SLC6A2 | ZBBX |
| BEST4 | KCTD8 | C6orf57 | BSN | C6orf115 | SIDT1 |
| SCAI | ATP6V1G2 | NREP | TLE2 | LINC00277 | RBP4 |
| ATP6V1G2 | LAMB3 | TRANK1 | DYDC2 | LOC100505576 | USP9Y |
| RAD51D | MAP3K9 | TMEM233 | EGFEM1P | OR1F1 | CRTAC1 |
| GPR26 | MFSD4 | EREG | ITGA8 | GNAL | DNAJC6 |
| ATOH7 | RASGRP1 | CYP4Z1 | FAM133A | GJD2 | PRPS2 |
| ITPR1 | MPP3 | CABP7 | TMEM145 | PRLHR | IPW |
| SYNJ1 | ASXL3 | PCDHAC2 | MDGA2 | CLEC4G | MYO5A |
| TMEM132D | C15orf38-AP3S2 | AJAP1 | ZNF273 | UNC13A | HYLS1 |
| ALK | FAM24B | B7H6 | GRIK3 | KIAA2022 | TMEM63C |
| ODZ3 | TC2N | CORO2A | ALDH1A2 | CCL13 | DPY19L2P1 |
| ATP6V1G2 | NPY5R | NECAB2 | TSPYL2 | RP1-177G6.2 | HPCAL1 |
| C1orf216 | INTS4L1 | HTR4 | GPR123 | GRIP2 | ARL15 |
| SLC17A7 | GPR6 | SPANXA1 | LARGE | PPP3CB | HUNK |
| LENG9 | NAALAD2 | SPANXA1 | PLEKHA5 | SLC4A8 | BHLHB9 |
| AP3M2 | ADARB1 | PPARGC1B | CACNA1D | CD8A | JPH4 |
| SLC25A12 | ATP2B1 | RAB15 | RASL11B | CALY | FKBP11 |
| LOC100128239 | NECAP1 | LRRTM1 | STMN1 | TSPYL4 | NKRF |
| B4GALNT1 | LOC100506123 | SUGT1P3 | CNIH2 | CD3D | AASDHPPT |
| ATP6V1A | LGALSL | CCDC65 | PRKAA2 | ARHGAP44 | LOC441455 |
| LONRF2 | SLC24A4 | ADAM23 | TNNT2 | EFHA2 | BBS7 |
| MANSC4 | SLC24A3 | CHML | ERBB4 | PDE8B | SCAND3 |
| LOC285593 | CGREF1 | IRS1 | PRR4 | FGF10 | MGC45800 |
| OXCT1 | GPLD1 | YPEL4 | NEU3 | HTR3A | LOC100506757 |
| ADCYAP1 | DPY19L2P4 | CAMK4 | C17orf69 | SYT14 | LRP11 |

neuron specific genes in mouse, gene numbers 209 onwards

| 1,050-1,084 | 1,085-1,119 | 1,120-1,154 | 1,155-1,189 | 1,190-1,224 |
|---|---|---|---|---|
| SYT2 | VDAC3 | TRPC3 | VSTM5 | KCNAB1 |
| CASQ1 | TMEM35 | LOC144481 | DISP2 | C18orf42 |
| OR2D3 | RPAIN | LOC201477 | PPM1H | PXDNL |
| SCAND3 | KLHDC8A | KBTBD6 | DNER | NTNG1 |
| MZT1 | PTER | KIF3A | TAF5 | LOC729177 |
| MORN4 | EIF4A2 | TDRD9 | LOC389493 | TMTC1 |
| KCND2 | AK5 | MACROD2 | HSPC072 | PJA1 |
| FAM126B | FAM169A | BLCAP | LOC729080 | NLGN1 |
| EPDR1 | NEFH | SRSF12 | PTS | MAPK9 |
| AGBL4 | DGKI | TRIB3 | AFAP1-AS1 | PDC |
| PLCE1 | OLA1 | SLC9A7 | C1orf114 | EEF1A2 |
| C1QL3 | BAG4 | MAP2 | LOC100507254 | LOC100379224 |
| C1QTNF9B | ADRA1B | MGC57346 | MEF2C | UPP1 |
| MARK1 | RANBP17 | RAB6B | MIAT | KLHL14 |
| KCNN2 | C1orf96 | FAM156A | HLA-F-AS1 | LRRTM4 |
| DOCK3 | SPANXA2-OT1 | LOC100126784 | RHEBL1 | PHTF1 |
| REM2 | ABCA9 | H2BFXP | EPHA6 | ZNF215 |
| TRH | MYOZ3 | KIAA1804 | KCNK2 | ANKRD32 |
| MYPOP | MAP1LC3A | ECE2 | KCNH2 | MAPT-AS1 |
| HCN4 | ZDHHC23 | PLCB1 | NEDD4L | SCN2B |
| RTN3 | AFAP1 | KIAA1383 | FLI43390 | SUB1 |
| TCTEX1D2 | SGTB | PDE10A | SERP2 | C14orf23 |
| LOC100130264 | LBH | MBLAC2 | AFF3 | SAMD5 |
| LRRC39 | ZDHHC22 | FABP6 | CMAS | GPR52 |
| PPP1R17 | FAM3C | LY86-AS1 | KALRN | NECAB1 |
| STBD1 | MYO5B | THBS1 | DCAF12L2 | SCN9A |
| CNRIP1 | LOC81691 | PPP1R2 | ANKRD50 | PIRT |
| FREM3 | HN1 | PIK3R3 | EEF1E1 | LOC730101 |
| CNTNAP4 | TFAMP1 | FAM71D | PVR | COLQ |
| JAZF1 | FAM189A1 | FSTL4 | PDF | PCDH20 |
| ATCAY | KIAA1586 | P2RX5 | CYP26B1 | AAK1 |
| PEG10 | FKBP1B | TRIM67 | LOC338817 | FAM71C |
| C16orf45 | SMPD3 | SETBP1 | ACOT7 | C7 |
| FBLN5 | TIAM2 | ATP2B2 | FAM196A | OSCP1 |

neuron specific genes in mouse, gene numbers 209 onwards

| 1,225-1,259 | 1,260-1,294 | 1,295-1,329 | 1,330-1,364 | 1,365-1,399 |
|---|---|---|---|---|
| SOX11 | TEX26-AS1 | ME3 | CHST15 | RAB9B |
| DPRX | LRRC66 | FLI41278 | GLRX2 | TACC2 |
| JPH3 | ABCC5 | UPP2 | TNFAIP8L1 | DCAF4 |
| APOA2 | TMEM182 | UQCRHL | ELK1 | DCC |
| MCOLN3 | ASPHD2 | BEND6 | LOC400940 | LMOD3 |
| ZNF428 | C5orf34 | ATPIF1 | PID1 | DNAH6 |
| UBE3D | DYNLT3 | PKI55 | ZNF667 | KHDC1L |
| UBE2N | TBC1D3F | GGH | CPLX1 | SRD5A1 |
| C1orf220 | SORBS2 | GALNT13 | ANO1 | SLITRK1 |
| DSCR9 | ZFP37 | R3HDM1 | RS1 | ALKBH8 |
| LOC729911 | C3orf26 | SLC25A26 | C9orf47 | NAGPA |
| PANK1 | RASAL2 | ASAH2B | LOC728392 | CLCN4 |
| GABRA6 | GSTA4 | FAN1 | GPR153 | ZNF697 |
| C5orf25 | VWDE | SLC22A3 | LOC100129961 | TMEM151B |
| WNT16 | PLCH1 | ZNF599 | HIST1H2AB | CCL1 |
| KCNH7 | NYAP1 | LOC400456 | MYO1B | CUZD1 |
| HS3ST5 | GPR162 | DKK1 | YEATS4 | OCRL |
| MAP2K4 | POSTN | LSM11 | KCNA1 | INTS4L2 |
| METTL21C | HSPA4L | HLF | ITFG1 | HENMT1 |
| AHSG | TMEM183A | CDR2 | DLG2 | DLEU2L |
| MYB | USP32P1 | EYS | AHI1 | CENPV |
| XKR6 | LOC100505738 | LOC646214 | HECW2 | SYCE2 |
| ZNF25 | KAZN | NCRNA00185 | LRRD1 | CDKN2AIPNL |
| SCOC | TASP1 | FAM110C | KIAA1549 | SCAMP1 |
| KCND3 | STAC2 | TAF9 | KCNIP1 | C9orf68 |
| LMTK2 | SNRPN | TBC1D9 | TAAR6 | FAM20A |
| CDKL5 | SLC1A6 | TRMT6 | COL19A1 | AP1S1 |
| ATP1A1 | B3GALNT1 | LOC728084 | ABCA10 | LOC139201 |
| OPN5 | KGFLP2 | OCM | CTPS | PTTG1 |
| FAM110B | ZNF829 | ZNF33B | KIRREL3 | PFDN6 |
| ADAMTS9-AS2 | NGFRAP1 | LYPD6 | FLT3 | PFDN6 |
| CPNE5 | ERLEC1 | ICA1 | RABL2B | PFDN6 |
| SOX11 | TEX26-AS1 | ME3 | CHST15 | RAB9B |
| DPRX | LRRC66 | FLJ41278 | GLRX2 | TACC2 |

neuron specific genes in mouse, gene numbers 209 onwards

| 1,400-1,434 | 1,435-1,469 | 1,470-1,504 | 1,505-1,539 | 1,540-1,573 |
|---|---|---|---|---|
| C3orf14 | ATG16L1 | MET | APOBEC3H | C6orf165 |
| RDH12 | RNF150 | TRAPPC6B | EID2B | C6orf228 |
| APOBEC4 | MKL2 | G3BP2 | ZNF280B | LYG1 |
| PATE2 | GPR88 | NME5 | GAS7 | GPC3 |
| LOC644242 | MORF4L2 | PCDHB3 | PPME1 | USMG5 |
| PRKACB | MEST | ODF2L | C6orf147 | PPP1R2P3 |
| FCRLB | CACNA1G | OPN3 | CDC42 | BCAT1 |
| ZDBF2 | PCMT1 | AKAP2 | SEMA3E | GRIA2 |
| FARSB | AIM2 | PACRG | TMEM106C | STOX2 |
| LINC00282 | C1orf94 | PFDN6 | SERINC1 | SUCLA2 |
| ANKRD20A2 | PPP1R14C | IER3IP1 | NDUFAF4 | MAPK13 |
| TAF7L | FAM83D | PEX10 | HSDL1 | SUZ12P |
| IL5RA | FBXO34 | NDN | XCL1 | FAM162B |
| FAM20B | SEMA4F | ABLIM2 | C14orf162 | DCBLD2 |
| PKIA | DGKB | GFPT1 | EPHA4 | SEC61G |
| FAM49A | DUSP28 | IL1RAPL1 | CAMK2D | SNX25 |
| LOC642852 | UCHL5 | HSF2 | TPTE2P1 | HDX |
| TTC9B | COX7A2L | ANO3 | MGC16142 | CDON |
| PHYHIP | FGF17 | NIPAL2 | CABYR | FAM78B |
| CYP2C8 | KIAA1377 | NDFIP1 | VN1R1 | ZNF248 |
| KCHK3 | TRIM37 | ANKRD20A4 | GOT2 | OR7E12P |
| SLC9A2 | CBWD3 | SH3BP5 | LOC284578 | DNAJB14 |
| C14orf2 | MKX | CHAF1B | NUDT21 | P4HA2 |
| FANCL | KIAA1199 | CDK5 | KIAA0528 | ATL1 |
| FAM18B2-CDRT4 | LOC728758 | SMYD2 | DCP2 | GYPA |
| KIF3C | EIF4E3 | LOC283177 | TMEM17 | AUH |
| STRBP | FLI41649 | LOC100131289 | DNAJC5G | TTC33 |
| MAS1 | CHIC1 | KIAA0825 | LIN7A | SPIN3 |
| IMMP2L | FBN1 | RNF41 | LOC286367 | C6orf165 |
| ANKRD20A2 | SAPCD1 | ADAMTSL3 | APOBEC3H | C6orf228 |
| YWHAB | SEMA4G | ADAMTSL1 | EID2B | LYG1 |
| PKNOX2 | BEX4 | CRHR2 | ZNF280B | GPC3 |
| C3orf14 | ATG16L1 | MET | GAS7 | USMG5 |
| RDH12 | RNF150 | TRAPPC6B | PPME1 | |

TABLE E
Key to Table D
Col. A-C in Table D are data derived from the published literature

| | | |
|---|---|---|
| Col. A | Transmembrane proteins detected | “Establishing the Proteome of Normal |
| | in cell-free CSF through mass | Human Cerebrospinal Fluid” Schutzer S E et |
| | spectrometry analysis. | al., PLoS One, 2010; 5(6): e10980. |
| | | This paper provides a list of proteins detected |
| | | through mass spectrometry analysis in cell- |
| | | free CSF. |
| Col. B | Neuron specific genes expressed in | “An RNA-Sequencing Transcriptome and |
| | mouse | Splicing Database of Glia, Neurons, and |
| | | Vascular Cells of the Cerebral Cortex” |
| | | Zhang Y et al., The Journal of Neuroscience, |
| | | 2014, 34(36): 11929-11947. |
| | | This paper compares gene expression in |
| | | different cells of the brain in mouse |
| Col. C | Neuron specific genes expressed in | “Purification and Characterization of |
| | human | Progenitor and Mature Human Astrocytes |
| | | Reveals Transcriptional and Functional |
| | | Differences with Mouse” Zhang et al., 2016, |
| | | Neuron 89, 37-53 |
| | | This paper compares gene expression in |
| | | different cells of the brain in human. |

Col. D-F in Table D are data obtained from intersection of the data from col. A-C

| | | |
|---|---|---|
| Col. D | CSF transmembrane proteins that | Intersect of (A, B) |
| | are neuron specific in mouse | |
| Col. E | CSF transmembrane proteins that | Intersect of (A, C) |
| | are neuron specific in human | |
| Col. F | CSF transmembrane proteins that | Intersect of (A, B, C) |
| | are neuron specific in mouse AND | |
| | human | |
| Col. G | Proteins found in neuron exosomes. | Data from the applicants obtained from |
| | | mass spectrometry analysis of IPSC-derived |
| | | neurons. |

Col. H-M in Table D are data obtained from intersection between data from the applicant
(col. G) and data derived from the published literature (Col. A-F).

| | | |
|---|---|---|
| Col. H | CSF transmembrane proteins | Intersect of (G, F) = intersect of (A, B, C, G) |
| | (detected through mass | |
| | spectrometry analysis) in neuron | |
| | exosomes | |
| Col. I | neuron specific genes in mouse | Intersect of (G, B) |
| | AND in neuron exosomes | |
| Col. J | neuron specific genes expressed in | Intersect of (G, C) |
| | human AND in neuron exosomes | |
| Col. K | CSF transmembrane proteins that | Intersect of (G, D) = intersect of (G, A, B) |
| | are neuron specific in mouse AND | |
| | in neuron exosomes | |
| Col. L | CSF transmembrane proteins that | Intersect of (G, E) = intersect of (G A, C) |
| | are neuron specific in human AND | |
| | in neuron exosomes | |
| Col. M | CSF transmembrane proteins that | Intersect of (K, L) = intersect of (G, A, B, C) |
| | are neuron specific in mouse AND | |
| | human AND in neuron exosomes | |

The present invention may be applied to genetic mutations further described in Genetic Diseases of the Eye, Second Edition, edited by Elias I. Traboulsi, Oxford University Press, 2012. Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

In some embodiments, the condition (disorder) may be neoplasia. In some embodiments, where the condition is neoplasia, the genes to be diagnosed, prognosed and/or targeted are any of those listed in Table A (in this case PTEN and so forth). In some embodiments, the condition may be Age-related Macular Degeneration. In some embodiments, the condition may be a Schizophrenic Disorder. In some embodiments, the condition may be a Trinucleotide Repeat Disorder. In some embodiments, the condition may be Fragile X Syndrome. In some embodiments, the condition may be a Secretase Related Disorder. In some embodiments, the condition may be a Prion-related disorder. In some embodiments, the condition may be ALS. In some embodiments, the condition may be a drug addiction. In some embodiments, the condition may be Autism. In some embodiments, the condition may be Alzheimer's Disease. In some embodiments, the condition may be inflammation. In some embodiments, the condition may be Parkinson's Disease.

Examples of proteins associated with Parkinson's disease include but are not limited to α-synuclein, DJ-1, LRRK2, PINK1, Parkin, UCHL1, Synphilin-1, and NURR1.

Examples of addiction-related proteins may include ABAT for example.

Examples of inflammation-related proteins may include the monocyte chemoattractant protein-1 (MCP1) encoded by the Ccr2 gene, the C-C chemokine receptor type 5 (CCR5) encoded by the Ccr5 gene, the IgG receptor IIB (FCGR2b, also termed CD32) encoded by the Fcgr2b gene, or the Fc epsilon R1g (FCER1g) protein encoded by the Fcer1g gene, for example.

Examples of cardiovascular diseases associated proteins may include IL1B (interleukin 1, beta), XDH (xanthine dehydrogenase). TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), or CTSK (cathepsin K), for example.

Examples of Alzheimer's disease associated proteins may include the very low density lipoprotein receptor protein (VLDLR) encoded by the VLDLR gene, the ubiquitin-like modifier activating enzyme 1 (UBA1) encoded by the UBA1 gene, or the NEDD8-activating enzyme E1 catalytic subunit protein (UBE1C) encoded by the UBA3 gene, for example.

Examples of proteins associated Autism Spectrum Disorder may include the benzodiazepine receptor (peripheral) associated protein 1 (BZRAP1) encoded by the BZRAP1 gene, the AF4/FMR2 family member 2 protein (AFF2) encoded by the AFF2 gene (also termed MFR2), the fragile X mental retardation autosomal homolog 1 protein (FXR1) encoded by the FXR1 gene, or the fragile X mental retardation autosomal homolog 2 protein (FXR2) encoded by the FXR2 gene, for example.

Examples of proteins associated Macular Degeneration may include the ATP-binding cassette, sub-family A (ABC1) member 4 protein (ABCA4) encoded by the ABCR gene, the apolipoprotein E protein (APOE) encoded by the APOE gene, or the chemokine (C-C motif) Ligand 2 protein (CCL2) encoded by the CCL2 gene, for example.

Examples of proteins associated Schizophrenia may include NRG1, ErbB4, CPLX1, TPH1, TPH2, NRXN1, GSK3A, BDNF, DISC1, GSK3B, and combinations thereof.

Examples of proteins involved in tumor suppression may include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4, for example.

Examples of proteins associated with a secretase disorder may include PSENEN (presenilin enhancer 2 homolog (C. elegans)), CTSB (cathepsin B), PSEN1 (presenilin 1), APP (amyloid beta (A4) precursor protein), APH1B (anterior pharynx defective 1 homolog B (C. elegans)), PSEN2 (presenilin 2 (Alzheimer disease 4)), or BACE1 (beta-site APP-cleaving enzyme 1), for example.

Examples of proteins associated with Amyotrophic Lateral Sclerosis may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins associated with prion diseases may include SOD1 (superoxide dismutase 1), ALS2 (amyotrophic lateral sclerosis 2), FUS (fused in sarcoma), TARDBP (TAR DNA binding protein), VAGFA (vascular endothelial growth factor A), VAGFB (vascular endothelial growth factor B), and VAGFC (vascular endothelial growth factor C), and any combination thereof.

Examples of proteins related to neurodegenerative conditions in prior disorders may include A2M (Alpha-2-Macroglobulin), AATF (Apoptosis antagonizing transcription factor), ACPP (Acid phosphatase prostate), ACTA2 (Actin alpha 2 smooth muscle aorta), ADAM22 (ADAM metallopeptidase domain), ADORA3 (Adenosine A3 receptor), or ADRA1D (Alpha-1D adrenergic receptor for Alpha-1D adrenoreceptor), for example.

Examples of proteins associated with Immunodeficiency may include A2M [alpha-2-macroglobulin]; AANAT [arylalkylamine N-acetyltransferase]; ABCA1 [ATP-binding cassette, sub-family A (ABC1), member 1]; ABCA2 [ATP-binding cassette, sub-family A (ABC1), member 2]; or ABCA3 [ATP-binding cassette, sub-family A (ABC1), member 3]; for example. Examples of proteins associated with Trinucleotide Repeat Disorders include AR (androgen receptor), FMR1 (fragile X mental retardation 1), HTT (huntingtin), or DMPK (dystrophia myotonica-protein kinase), FXN (frataxin), ATXN2 (ataxin 2), for example.

Examples of proteins associated with Neurotransmission Disorders include SST (somatostatin), NOS1 (nitric oxide synthase 1 (neuronal)), ADRA2A (adrenergic, alpha-2A-, receptor), ADRA2C (adrenergic, alpha-2C-, receptor), TACR1 (tachykinin receptor 1), or HTR2c (5-hydroxytryptamine (serotonin) receptor 2C), for example.

Examples of neurodevelopmental-associated sequences include A2BP1 [ataxin 2-binding protein 1], AADAT [aminoadipate aminotransferase], AANAT [arylalkylamine N-acetyltransferase], ABAT [4-aminobutyrate aminotransferase], ABCA1 [ATP-binding cassette, sub-family A (ABC1), member 1], or ABCA13 [ATP-binding cassette, sub-family A (ABC1), member 13], for example.

Further examples of preferred conditions treatable with the present system include may be selected from: Aicardi-Goutières Syndrome; Alexander Disease; Allan-Herndon-Dudley Syndrome; POLG-Related Disorders; Alpha-Mannosidosis (Type II and III); Alström Syndrome; Angelman; Syndrome; Ataxia-Telangiectasia; Neuronal Ceroid-Lipofuscinoses; Beta-Thalassemia; Bilateral Optic Atrophy and (Infantile) Optic Atrophy Type 1; Retinoblastoma (bilateral); Canavan Disease; Cerebrooculofacioskeletal Syndrome 1 [COFS1]; Cerebrotendinous Xanthomatosis; Cornelia de Lange Syndrome; MAPT-Related Disorders; Genetic Prion Diseases; Dravet Syndrome; Early-Onset Familial Alzheimer Disease; Friedreich Ataxia [FRDA]; Fryns Syndrome; Fucosidosis; Fukuyama Congenital Muscular Dystrophy; Galactosialidosis; Gaucher Disease; Organic Acidemias; Hemophagocytic Lymphohistiocytosis; Hutchinson-Gilford Progeria Syndrome; Mucolipidosis II; Infantile Free Sialic Acid Storage Disease; PLA2G6-Associated Neurodegeneration; Jervell and Lange-Nielsen Syndrome; Junctional Epidermolysis Bullosa; Huntington Disease; Krabbe Disease (Infantile); Mitochondrial DNA-Associated Leigh Syndrome and NARP; Lesch-Nyhan Syndrome; LIS1-Associated Lissencephaly; Lowe Syndrome; Maple Syrup Urine Disease; MECP2 Duplication Syndrome; ATP7A-Related Copper Transport Disorders; LAMA2-Related Muscular Dystrophy; Arylsulfatase A Deficiency; Mucopolysaccharidosis Types I, II or III; Peroxisome Biogenesis Disorders, Zellweger Syndrome Spectrum; Neurodegeneration with Brain Iron Accumulation Disorders; Acid Sphingomyelinase Deficiency; Niemann-Pick Disease Type C; Glycine Encephalopathy; ARX-Related Disorders; Urea Cycle Disorders; COL1A1/2-Related Osteogenesis Imperfecta; Mitochondrial DNA Deletion Syndromes; PLP1-Related Disorders; Perry Syndrome; Phelan-McDermid Syndrome; Glycogen Storage Disease Type II (Pompe Disease) (Infantile); MAPT-Related Disorders; MECP2-Related Disorders; Rhizomelic Chondrodysplasia Punctata Type 1; Roberts Syndrome; Sandhoff Disease; Schindler Disease—Type 1; Adenosine Deaminase Deficiency; Smith-Lemli-Opitz Syndrome; Spinal Muscular Atrophy; Infantile-Onset Spinocerebellar Ataxia; Hexosaminidase A Deficiency; Thanatophoric Dysplasia Type 1; Collagen Type VI-Related Disorders; Usher Syndrome Type I; Congenital Muscular Dystrophy; Wolf-Hirschhorn Syndrome; Lysosomal Acid Lipase Deficiency; and Xeroderma Pigmentosum.

Some examples of disorders (conditions or diseases) that might be usefully treated, prognosed and/or diagnosed using the present invention are included in the Tables above and examples of genes or markers currently associated with those disorders are also provided there. However, the genes exemplified are not exhaustive.

In one aspect, the invention provides kits containing any one or more of the elements disclosed in the above methods and compositions. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In some embodiments, the kit comprises one or more of the vectors and/or one or more of the polynucleotides described herein. The kit may advantageously allows to provide all elements of the systems of the invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Isolation/Purification of Exosomes, and RNA Extraction Therefrom (No Proteinase Treatment): Standard Exosome Isolation

The following protocol was used to isolate RNA from suspension cells such as K562 Cells. Buffers and some reagents refer to a mirVana RNA kit (Life technologies).

Day 1
Spin down about 72 million cells total in 6 50 mL Falcon tubes (12 million cells per tube) at 300×g for 5 minutes.
Aspirate media and resuspend each cell pellet in 43 mL exosome-free media. Transfer contents of each Falcon tube to T75 flask.

Day 2
After 24 hours, take off all media and divide among 50 mL falcon tubes. Spin at 300×g for 10 minutes at 4 degrees.
Transfer supernatant to new 50 mL tubes leaving cell pellet behind. Spin at 2000×g for 10 minutes at 4 degrees. Transfer supernatant to new 50 mL tubes leaving cell pellet behind.
Spin supernatant at 16,500×g for 20 minutes at 4 degrees.
Transfer supernatant to new 50 mL tubes, leaving pellet behind.
Pass supernatant through Steriflip 0.22 micron filter.
Transfer supernatant to pollyallomer ultracentrifuge tubes. Centrifuge at 120,000×g (26,500 RPM with SW32Ti rotor) for 70 minutes at 4 degrees.
Remove supernatant, leaving ˜2 cm of media above pellet. Add 5 mL PBS to each tube. Vortex on medium speed for a few seconds. Fill to top of each tube with PBS.
Again, centrifuge at 120,000×g for 70 minutes at 4 degrees.
Aspirate all of supernatant with Pasteur pipet without touching bottom of tube (where pellet is located).
Add 2 μL Superasin to each tube (SUPERase● In™ RNase Inhibitor from Life technologies).
Add 200 μL of Lysis/Binding Solution directly to the bottom of each ultracentrifuge tube. Pipet up and down. Transfer the contents of 3 ultracentrifuge tubes to one 1.5 mL Eppendorf tube.
Vortex briefly and place on ice.
Add 60 μL of miRNA Homogenate Additive to each tube ( 1/10 volume of lysate).
Vortex each tube and place on ice for 10 minutes.
Add a 600 μL of Acid-Phenol:Chloroform to each tube (volume that is equal to lysate volume before addition of miRNA Homogenate Additive).
Vortex for 30 seconds to mix thoroughly.
Centrifuge at maximum speed for 5 minutes (all spins at room temperature).
While tubes are spinning, transfer some Elution Solution to new 1.5 mL tube and pre-heat Elution Solution in heating block to 95° C. Also, put filter cartridges into collection tubes.
Carefully remove the upper (aqueous) phase and transfer to a new 1.5 mL tube.
Add 1.25 volumes of 100% ethanol to the transferred aqueous phase.
Pipet up and down and transfer up to 700 μL to a filter cartridge. Centrifuge at 10,000 RCF (10,000 RPM) for 15-30 seconds.
Discard flow-through and load the rest of the lysate/ethanol mixture. Centrifuge at 10,000 RCF (10,000 RPM) for 15-30 seconds.
Add 700 μL of miRNA Wash Solution 1 to filter and centrifuge at 10,000 RCF (10,000 RPM) for 15 seconds. Discard flow-through.
Add 500 μL of miRNA Wash Solution 2/3 to filter and centrifuge at 10,000 RCF (10,000 RPM) for 15 seconds. Discard flow-through.
Again, add 500 μL of miRNA Wash Solution 2/3 to filter and centrifuge at 10,000 RCF (10,000 RPM) for 15 seconds. Discard flow-through.
Put filter back in collection tube and spin for 1 minute at 10,000 RCF (10,000 RPM) to remove any residual ethanol from the filter.
Transfer filter cartridge with bound RNA to a new collection tube.
Add 100 μL of pre-heated Elution Buffer to the center of each filter. Centrifuge for 30 seconds at maximum speed to recover the RNA.
Store RNA at −80° C.

### Example 2: Isolation/Purification of Exosomes, and RNA Extraction Therefrom (with Proteinase and RNase Treatment): Removal of Protein-RNA Complexes from the Exosome Pellet

The following protocol was used to isolate RNA from suspension cells such as K562 Cells. This protocol removes RNA-protein complexes from the exosomes. Buffers refer to a mirVana RNA kit (Life technologies).
Execute exosome isolation protocol (see example 1) on 6×12 million cells up to the end of first ultracentrifugal spin.
Take off complete supernatant of all six tubes. Resuspend each in 150 μL PBS. Label two tubes P1 and P2 and to these, add 5 uL of proteinase K (active conc. 500 μg/mL).
Incubate all tubes at 37° C. for 30 minutes.
Fill tubes with PBS and ultracentrifuge again.
After second spin, take off complete supernatant of all six tubes. Resuspend each in 150 μL PBS. Label the four unlabeled tubes NT1, NT2, PR1 and PR2.
Add 5 μL of proteinase K (active conc. 500 μg/mL) to PR1 and PR2.
Incubate all tubes at 37° C. for 30 minutes.
Add 5 μL PMSF (from 20 mM stock; active conc. 1 mM) to PR1 and PR2.
Leave all tubes at RT for 10 minutes.
Add 0.5 μL RNase A/T1 (active conc. ˜3 μg/mL) to PR1 and PR2.
Incubate all tubes at 37° C. for 30 minutes.
Add 2 μL superasin to each tube. (SUPERase● In™ RNase Inhibitor from Life technologies).
Move contents of each tube to 1 Eppendorf tube (total volume should be ˜200 μL per tube due to residual liquid in UC tube), labeled accordingly, and proceed with mirVana RNA isolation using 300 μL lysis buffer.

### Example 3: Chemical and Enzymatic Treatment of Exosomes

To achieve purified exosomes which are essentially free of extra-exosomal nucleic acid-protein complexes, the following procedure is provided. In sum, DNase is added during the preparation, then inactivated prior to lysing all of the vesicles which affords a composition which is essentially free of extra-exosomal nucleic acid-protein complexes. Briefly, exosome pellet—either at the wash step between ultracentrifugations or after the final ultracentrifugation, as indicated—was resuspended in 50-500 μL PBS or 0.5% Triton X-100 as indicated. For proteinase treatment, Proteinase K (Life Technologies) was added to a final concentration of 500 μg/mL, and samples were incubated at 37° C. for 30 minutes. Treatment was initially done in Proteinase K activity buffer (0.1 M NaCl, 10 mM Tris pH 8, 1 mM EDTA) rather than PBS, however reduced RNA yields from untreated exosomes resuspended in this buffer were observed; thus, all further treatments were performed in PBS. Proteinase was subsequently inactivated by the addition of phenylmethylsulfonyl fluoride (PMSF; Millipore) to 1 mM concentration. For RNase treatment, RNase Cocktail Enzyme Mix (Life Technologies) was added to a final concentration of 1.25 and 50 U/mL RNase A and T1, respectively, and samples incubated at 37° C. for 30 minutes. RNase was inactivated by the addition of SUPERasein (Life Technologies) to 20 U/mL concentration and the addition of ≧2 volumes lysis buffer from mirVana miRNA isolation kit (Life Technologies). For DNase treatment, Turbo DNase (Life Technologies) was added to a concentration of 26 U/mL, with Turbo DNase buffer added to 1× concentration where indicated, and samples incubated at 37° C. for 30 minutes. DNase was inactivated by the addition of EDTA to 15 mM followed by incubation at 75° C. for 10 minutes.

### Example 4: CD81 and CD63 Exosome Isolation with Mouse IgG Beads (Pull Down Purification)

Day 0 (or Earlier)

1. Mix 50 mL FBS with 500 mL IMDM and 5 mL P/S. Filter through 0.22 μM filter. Grow cells.

Day 1

2. Spin down 72 million cells total in 6 50 mL Falcon tubes at 300×g for 5 minutes.
3. Aspirate media and resuspend each cell pellet in 43 mL AIM-V. Transfer contents of each Falcon tube to T75 flask.

Day 2

4. After 24 hours, take off all media and divide among 50 mL falcon tubes. Spin at 300×g for 10 minutes at RT.
5. Transfer supernatant to new 50 mL tubes leaving cell pellet behind. Spin at 2000×g for 10 minutes at RT. Transfer supernatant to new 50 mL tubes leaving cell pellet behind.
6. Spin supernatant at 16,500×g for 20 minutes at 4 degrees.
7. Transfer supernatant to new 50 mL tubes, leaving pellet behind.
8. Pass supernatant through Steriflip 0.22 micron filter.
9. Transfer supernatant to pollyallomer ultracentrifuge tubes. Centrifuge at 120,000×g (26,500 RPM with SW32Ti rotor) for 70 minutes at 4 degrees.
10. During this spin, make fresh Isolation Buffer (PBS supplemented with 1 mg/mL BSA, filtered through 0.22 m filter) and prepare hot plate at 95° C.
11. Also during first ultracentrifuge spin, prepare beads:
a. resuspend anti-mouse IgG Dynabeads by mixing for >10 min or vortexing gently for 30 s.
b. transfer 1001±L (4×107) beads each into 3 different Biotix 2 mL tubes labelled C, 81 and 63.
c. wash the magnetic beads by adding 1 mL of Isolation Buffer. Mix well.
d. place tubes on the magnet for 2 minutes and remove supernatant carefully.
e. remove tubes from magnet and add 100 μL isolation buffer.
f. To 81, add 20 μL (4 μg) anti-human CD81 antibody, clone 1.3.3.22
g. To 63, add 8 μL (4 μg) anti-human CD63 antibody, clone h5c6
h. To C, add 4 μL (4 μg) ctrl antibody (mouse mAb mCherry, 1C51)
i. Incubate on rotating rack in cold room until end of isolation (˜3 hours)
   12. Remove supernatant, leaving ˜2 cm of media above pellet. Add 5 mL PBS to each tube. Vortex on medium speed for a few seconds. Fill to top of each tube with PBS.
   13. Again, centrifuge at 120,000×g for 70 minutes at 4 degrees.
   14. Aspirate all of supernatant with Pasteur pipet without touching bottom of tube (where pellet is located).
   15. Add 80 μL PBS to each tube and let sit for ˜15 minutes.
   16. Resuspend and pool all tubes into a biotix tube labelled P. Measure total volume, should be ˜600 μL due to 20 μL residual liquid after aspiration.
   17. Retrieve bead tubes from cold room, spin briefly and place on magnet.
   18. Do 2×900 μL washes in isolation buffer to remove excess antibody.
   19. Split pooled pellets ⅙ into each of the biotix tubes. Add isolation buffer to each bead tube to 200 μL total volume. Put all on rotating rack in cold room overnight (16 hours)
   20. Add 33 μL 4× SB (133 μL total volume) to remaining 100 μL of exosomes in P and boil at 95° for 5 min. Place immediately on ice and freeze at −80°

Day 3

21. After 16 h, centrifuge all tubes from cold room briefly to collect samples.
22. Place C, 81 and 63. on magnet for two minutes. Collect supernatants and store in new tubes labelled C-FT, 81-FT, 63-FT respectively.
23. Wash beads in each tube with 500 μL Isolation Buffer. Leave 2 min on magnet before collecting wash supernatants. Add each wash to respective FT tube. Store at 4° C.
24. Add 133 μL 1× Sample Buffer to C, 81 and 63. and boil at 95° for 5 min. Place immediately on ice for 5 min, then place on magnet for 2 min, collect supernatants and freeze at −80° in new tubes.
25. Assemble all flow-through tubes and add each to its own ultracentrifuge tube. Fill tubes with PBS and spin 180 minutes at 120000 g.
26. After ultracentrifugation, remove supernatant entirely, add 80 μL PBS and leave pellets for ˜15 minutes.
27. Resuspend (should be about 100 μL) move to labelled biotix tubes and add 33 μL 4× Sample Buffer to each.
28. Boil at 95° C. for 5 min. Place immediately on ice and freeze at −80° C.

### Example 5: Analysis of RNA Contents of Exosomes as a Function of Exosome Purification Method Size Distribution

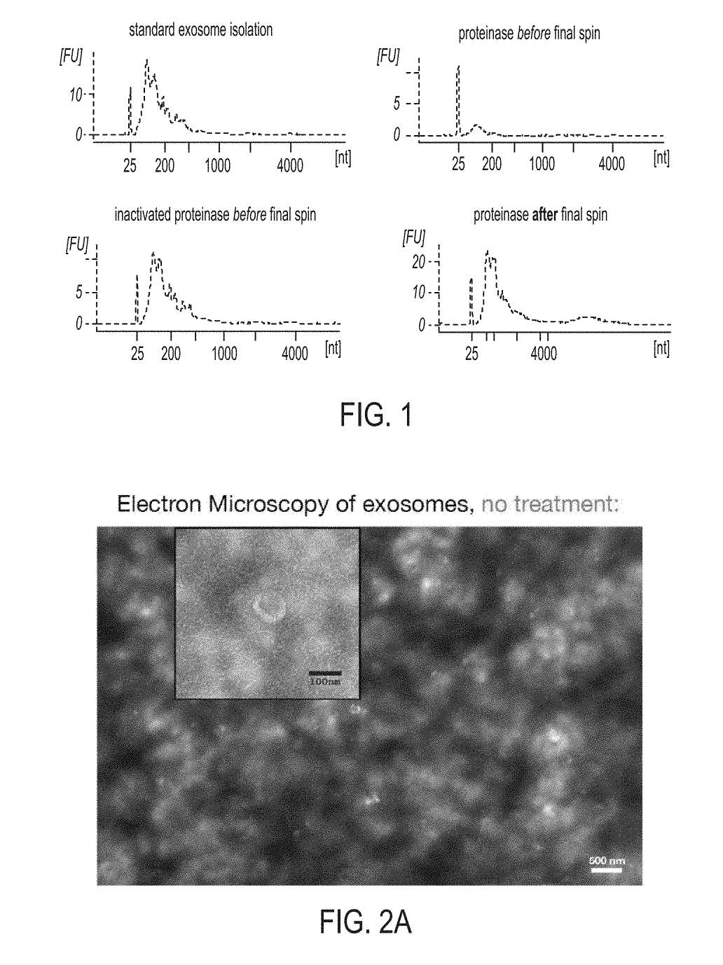

FIG. 1 shows graph of RNA fluorescence unit (FU) plotted against RNA size (nt), wherein “final spin” refers to the final centrifugation step.

The results allow comparison and validation of corresponding purification methods.

### Example 6: Analysis of RNA Contents of Exosomes as a Function of Exosome Purification Method—Electron Microscopy Imaging

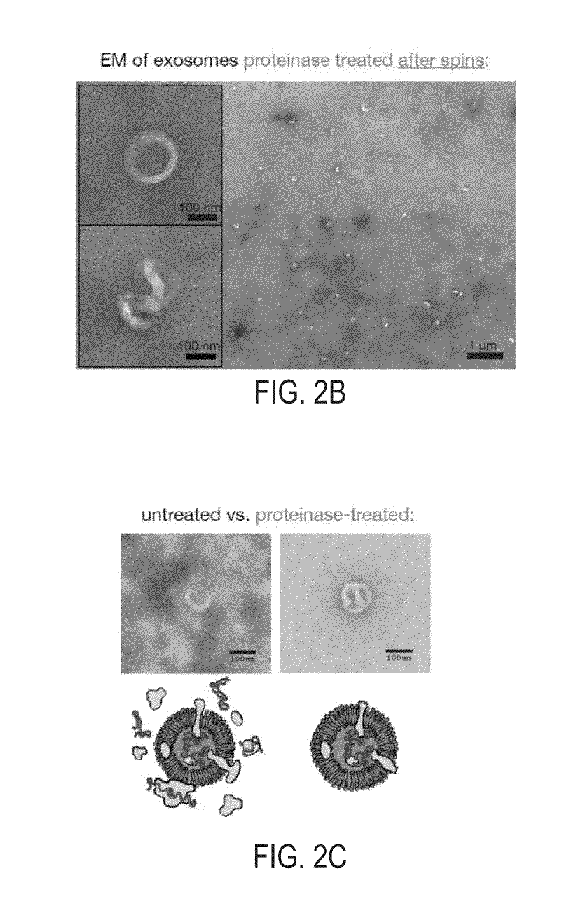

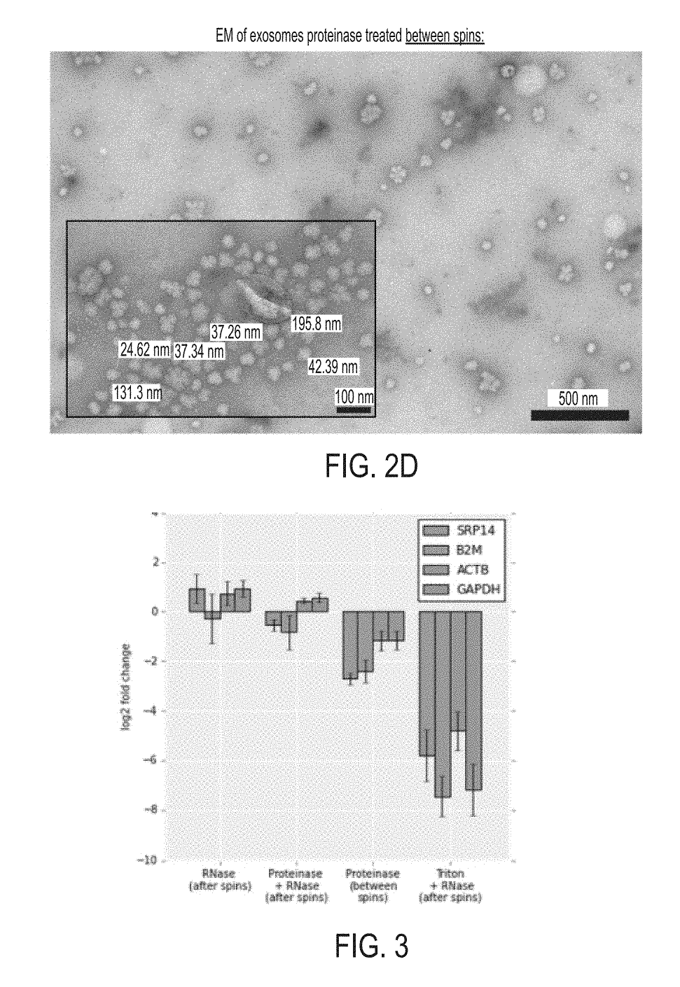

FIGS. 2A-D show electron microscopy (EM) photographs of exosome preparations, wherein “no treatment” refers to a protocol according to example 1; “after spins” refers to a protocol according to example 2; “between spins” denotes a protocol according to example 1, except that additional proteinase treatment occurred between the two ultracentrifugation steps.

The results show that the method used for exosome preparation affects exosome integrity. EM data allow comparison and validation of exosome purification methods.

Vesicles Electron Microscopy Prep

### Stain Prep

Weigh 60 mg powdered Uranyl Formate into clean 10 mL beaker with stir stick in radioactivity hood.
Move this to the stir plate (make sure stirring is OFF) and cover with the big beaker with tin foil.
Fill another clean 10 mL beaker with 3 mL water and heat this up (not on the same hot plate) until it's super boiling/as hot as possible. Ensure not to lose too much water to evaporation.
Quickly pour this into other beaker with powder and start stirring. Stir vigorously for 2 minutes protected by tin foil.
Using BD 5 mL syringe (with black lining inside, not the normject ones) suck up stain and then using coming 0.45 μm filter to filter it, deposit into 15 mL falcon tube. Label and wrap in tin foil.
Wipe beaker with Kim wipe. throw this, gloves, syringe and filter into radioactive waste

### Sample Prep

Good sample concentration is in the range of 1 nM
Use special tweezers to put grids on parafilm-covered slide, dark shiny side up.
Put slide in glow discharger, close lid carefully, hit start.
Pick up grids with tweezers at the edge, don't pinch too hard. Put tweezers down (still holding grid) and pipet 3.5 μL of sample onto it. Leave 1 minute. This time changes depending on salt concentration etc.
Wick away liquid with a piece of filter paper
Add 3.5 μL stain, leave 30 seconds, then wick away this as well.
Find a holder and carefully put grids down with dark side up, use this to carry to EM room

### Example 7: Analysis of RNA Contents of Exosomes as a Function of Exosome Purification Method—qRT-PCR Analysis—Validation of the Purification Method

FIG. 3 shows qRT-PCR data of exosome RNA for 4 mRNAs that were previously found in exosome RNA-Seq data.

The qRT-PCR is performed for various conditions of exosome purification methods. All runs are normalized to RNA from the ‘regular’ exosome isolation (Example 1). The conditions for exosome purification are as follows:

(1) RNase Treatment Only

(which is expected not be sufficient if RNA is also protected by proteins as was shown for extracellular microRNAs in Arroyo et al, Proc Natl Acad Sci USA. 2011 Mar. 22; 108(12):5003-8. doi: 10.1073/pnas. 1019055108. Epub 2011 Mar. 7, 2011; Turchinovich et al Nucleic Acids Res. 2011 Sep. 1; 39(16):7223-33. doi: 10.1093/nar/gkr254. Epub 2011 May 24.)

(2) Proteinase+RNase Treatments after Spins

(protocol as per Example 2; also see below)

(3) Proteinase Treatment (Between Spins)

This is the method described in previous publications such as Valadi et al, Nat Cell Biol. 2007 June; 9(6):654-9. Epub 2007 May 7. As shown by EM (see above and FIG. 2d), this method compromises exosome integrity.
In accordance with the EM data, the qRT-PCR results show a decrease in mRNA levels.

(4) Triton+RNase Treatments

This is a control run, wherein where Triton treatment is used to break open the vesicles, and samples are further treated with RNase. The results show drastic reduction in levels of mRNA.

R/T Isolation for qPCR
1. Execute exosome isolation protocol on 6×12 million cells up to the end of second ultracentrifugal spin.
2. Take off complete supernatant of all six tubes.
3. Resuspend 4 pellets in 150 μL PBS. Label them NT1, NT2, R1 and R2.
4. Resuspend other 2 pellets in 3% Triton. Label them TR1 and TR2,
5. Add 0.5 μL RNase A/T1 (active conc. ˜3 μg/mL) to R1, R2, TR1 and TR2,
6. Incubate all tubes at 37° C. for 30 minutes.
7. Add 2 μL superasin to each tube (SUPERase● In™ RNase Inhibitor from Life technologies).
8. Proceed with mirVana RNA isolation using 300 μL lysis buffer (mirVana RNA kit from Life technologies).

P/R Isolation for qPCR
1. Execute exosome isolation protocol on 6×12 million cells up to the end of first ultracentrifugal spin.
2. Take off complete supernatant of all six tubes. Resuspend each in 150 μL PBS. Label two tubes P1 and P2 and to these, add 5 μL of proteinase K (active conc. 500 μg/mL).
3. Incubate all tubes at 37° C. for 30 minutes.
4. Fill tubes with PBS and ultracentrifuge again.
5. After second spin, take off complete supernatant of all six tubes. Resuspend each in 150 μL PBS. Label the four unlabeled tubes NT1, NT2, PR1 and PR2.
6. Add 5 uL of proteinase K (active conc. 500 μg/mL) to PR1 and PR2.
7. Incubate all tubes at 37° C. for 30 minutes.
8. Add 5 μL PMSF (from 20 mM stock; active conc. 1 mM) to PR1 and PR2.
9. Leave all tubes at RT for 10 minutes.
10. Add 0.5 μL RNase A/T1 (active conc. ˜3 μg/mL) to PR1 and PR2.
11. Incubate all tubes at 37° C. for 30 minutes.
12. Add 2 μL superasin to each tube (SUPERase● In™ RNase Inhibitor from Life technologies).
13. Proceed with mirVana RNA isolation using 300 μL lysis buffer (mirVana RNA kit from Life technologies).

qRT-PCR
1. After collecting cell and exosome RNA, dilute 100 ng cell RNA to 100 μL with H₂O. Add 2 μL Turbo DNase (Lifetech), 2 μL Superasin, and 10 μL DNase buffer to each sample.
2. Incubate at 37° C. for 30 minutes.
3. Clean and concentrate using Zymo RNA Clean and Concentrate kit according to instructions and elute in 16 μL H₂O.
4. Perform reverse transcription using Superscript VILO cDNA Synthesis kit with 14 μL of RNA in a 20 μL reaction.
5. Perform qPCR using KAPA Fast qPCR SYBR mix (KAPA Biosystems) with 2 μL of cDNA per reaction.

The following primers were used:

| (SEQ ID NO: 1) | |
|---|---|
| SRP14-F | GGGTACTGTGGAGGGCTTTG |
| (SEQ ID NO: 2) | |
| SRP14-R | AGGAGGTTTGAATAAGCCATCTGA |
| (SEQ ID NO: 3) | |
| B2M-F | GTATGCCTGCCGTGTGAAC |
| (SEQ ID NO: 4) | |
| B2M-R | AAAGCAAGCAAGCAGAATTTGG |
| (SEQ ID NO: 5) | |
| ACTB-F | CGGCATCGTCACCAACTG |
| (SEQ ID NO: 6) | |
| ACTB-R | AACATGATCTGGGTCATCTTCTC |
| (SEQ ID NO: 7) | |
| GAPDH-F | GGTGGTCTCCTCTGACTTCAACA |
| (SEQ ID NO: 8) | |
| GAPDH-R | GTTGCTGTAGCCAAATTCGTTGT |

### Example 8: Correlation of Exosomal RNA Content with Cellular RNA Content

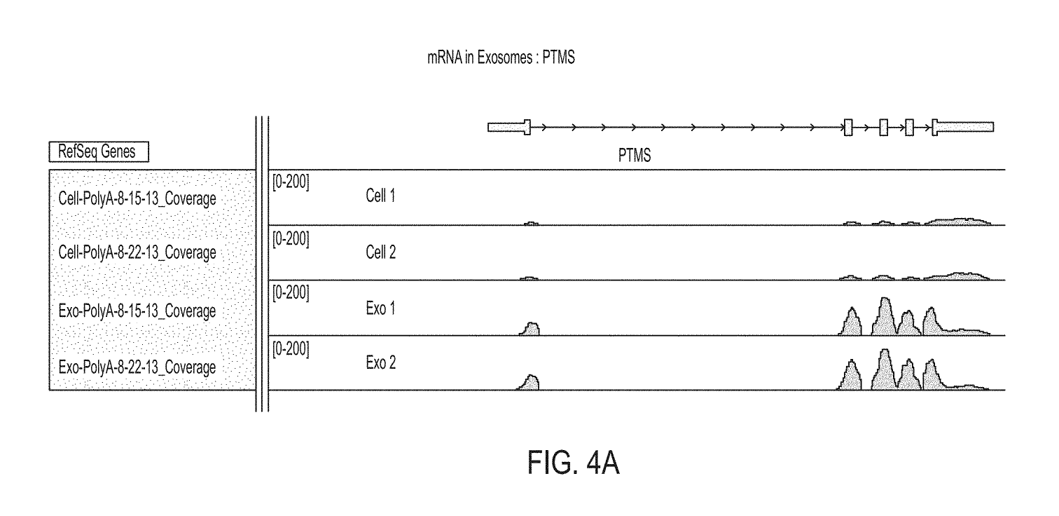

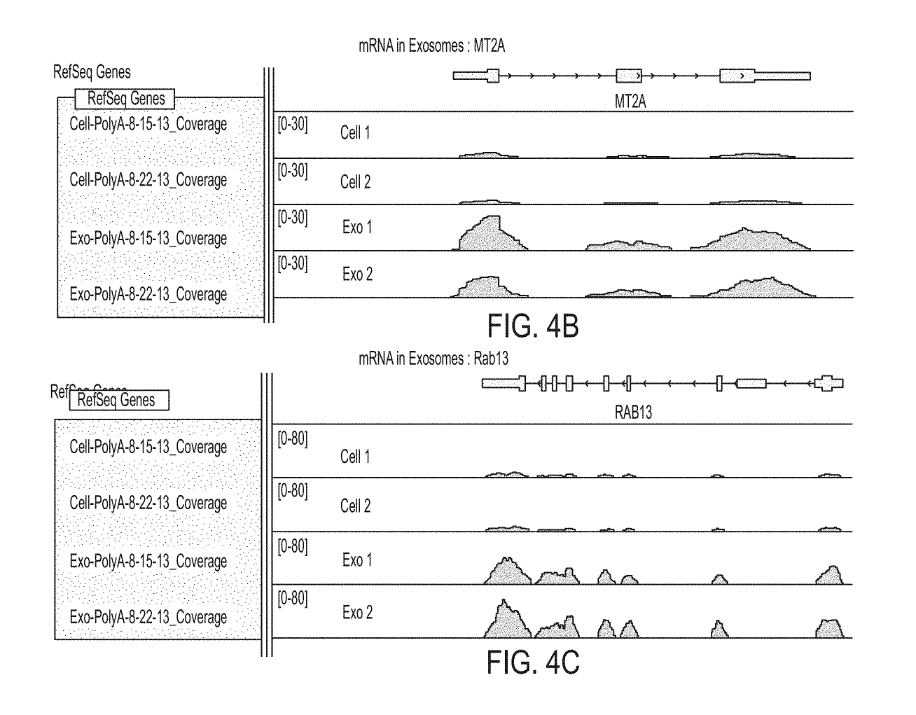

FIGS. 4A-C show RNA-Seq data, showing that the RNA profile of mRNAs in exosomes reflects that of the donor cells. This indicates that the exosomes provide an accurate snapshot of the transcriptome of the cells they come from. Exosome preparation was according to the standard exosome isolation procedure (as in Example 1, without proteinase/RNase).

RNA-Seq
1. After collecting cell and exosome RNA, dilute 100 ng cell RNA to 100 μL with H₂O. Add 2 μL Turbo DNase (Lifetech), 2 μL Superasin, and 10 μL DNase buffer to each sample.
2. Incubate at 37° C. for 30 minutes.
3. Clean and concentrate using Zymo RNA Clean and Concentrate kit according to instructions and elute in 10 μL H₂O.
4. Perform a PolyA Selection using Dynabeads mRNA Purification kit (Lifetech).
5. Proceed with RNA-Seq library prep protocol as described in: Perturbation of m6A writers reveals two distinct classes of mRNA methylation at internal and 5′ sites. Schwartz S, Mumbach M R, Jovanovic M, Wang T, Maciag K, Bushkin G G, Mertins P, Ter-Ovanesyan D, Habib N, Cacchiarelli D, Sanjana N E, Freinkman E. Pacold M E, Satija R, Mikkelsen T S, Hacohen N, Zhang F, Carr S A, Lander E S, Regev A. Cell Rep. 2014 Jul. 10; 8(1):284-96. doi: 10.1016/j.celrep.2014.05.048. Epub 2014 Jun. 26.

### Example 9: Exosome-Mediated RNA Transfer Experiment Between HEK293 and K562 Cells

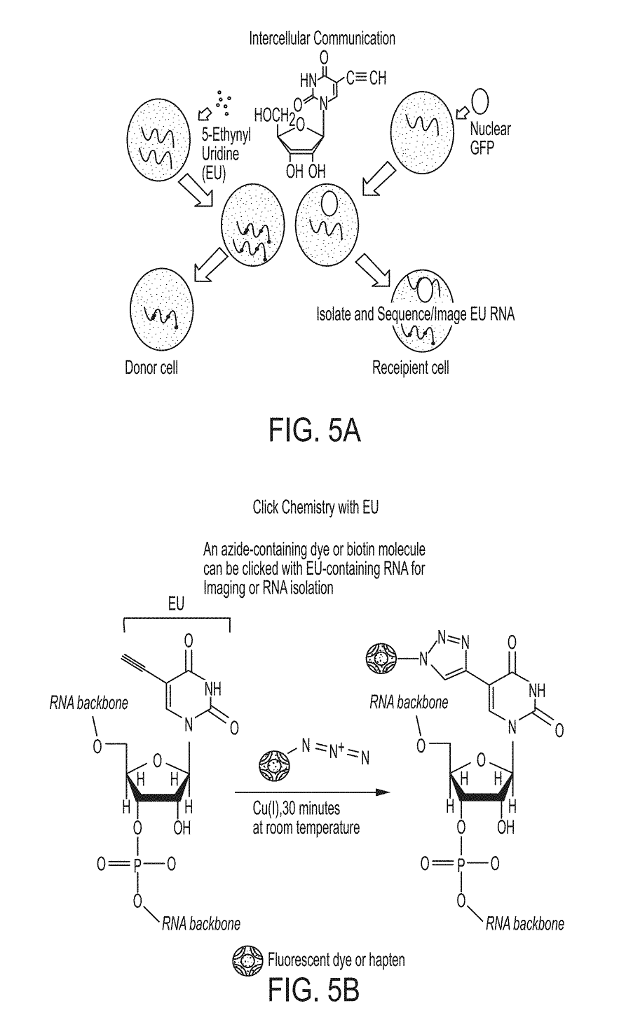

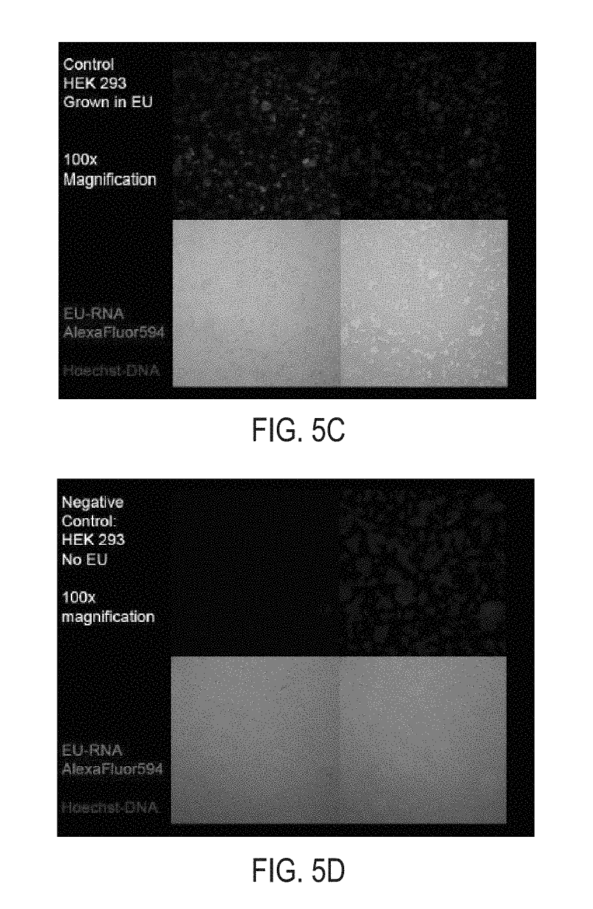

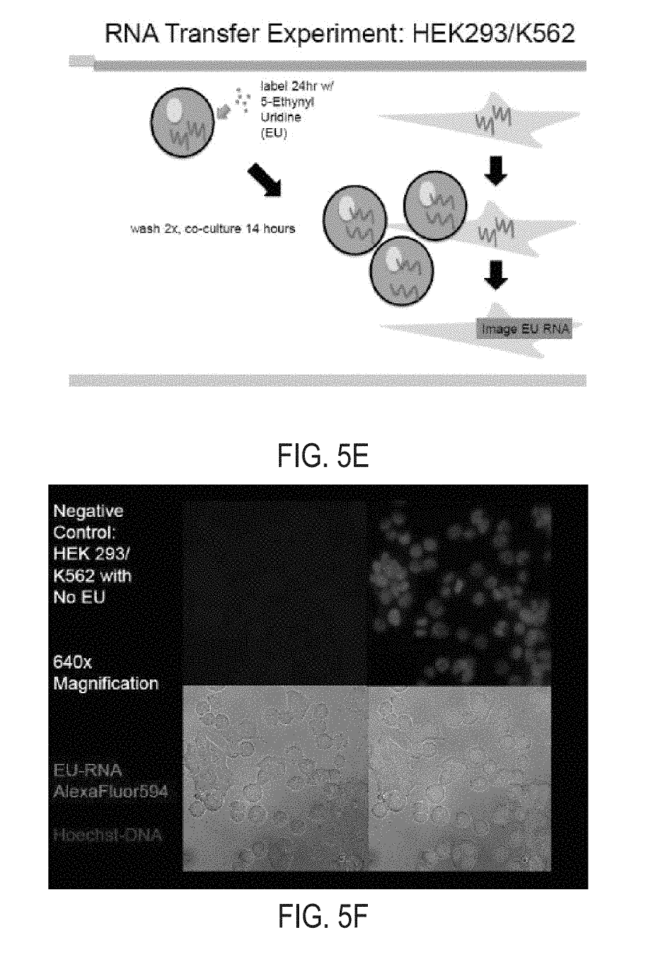

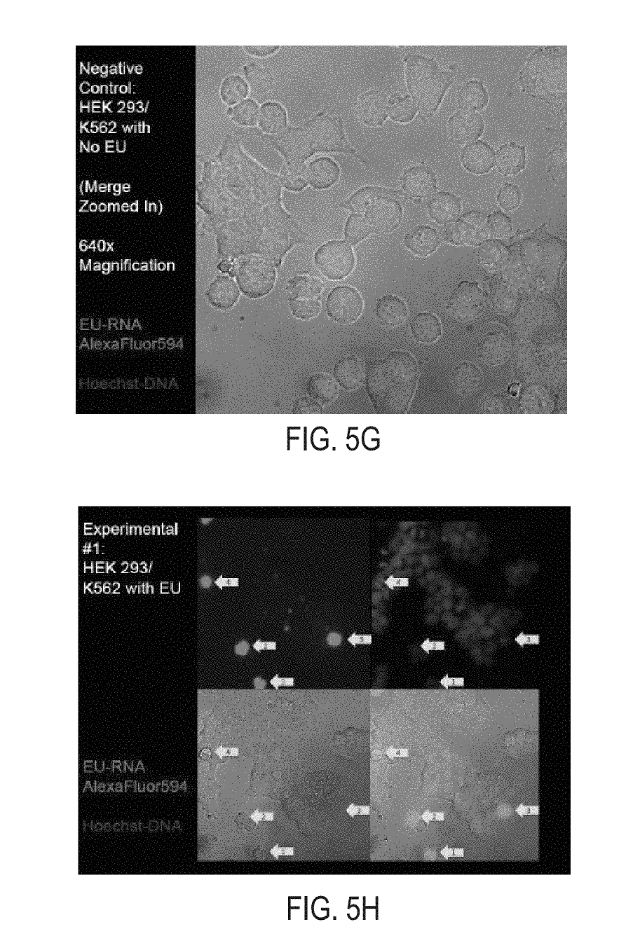

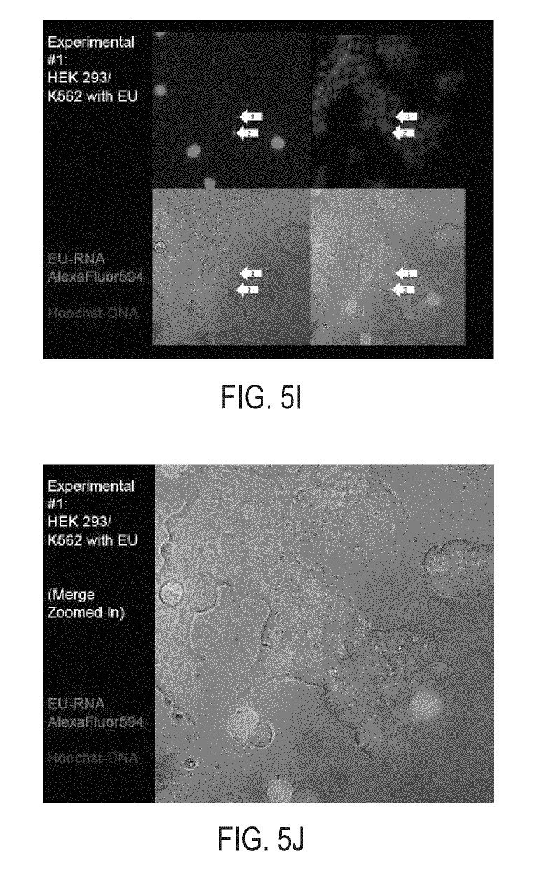

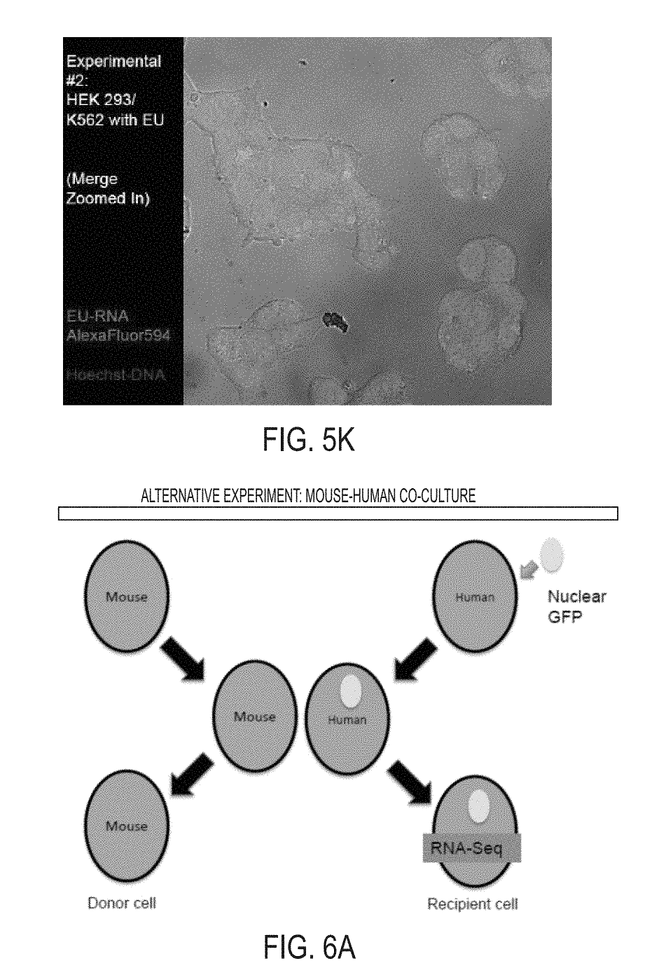

FIGS. 5A-K show fluorescence imaging of cells using EUclick chemistry.

This example shows results from a system that allows detection of potential endogenous RNA transfer between cells in a co-culture system by feeding donor cells with a modified nucleotide (5-ethynyl uridine, EU) that gets incorporated into its RNA and then co-culturing donor cells with unlabeled acceptor cells.

Click Chemistry is then used to detect RNA with the modified nucleotides by conjugate of a fluorophore to the EU. These results suggest the presence of RNA transfer. The white arrows point to spots of transferred RNA in the HEK293 acceptor cells. The green arrows just show the donor K562 cells.

EU-RNA Transfer Experiments

K562 and HEK293 cells were both obtained from ATCC.

K562 cells were incubated with 5-ethynyl uridine (Lifetech) diluted to 2 mM for 24 hours.

K562 and HEK 293 cells were co-cultured for 24 hours.

Cells were imaged using Click-IT RNA Alexa Fluor 594 Imaging kit (Lifetech)

### Example 10: Exosome-Mediated RNA Transfer Experiment Between Co-Cultured Cell Lines

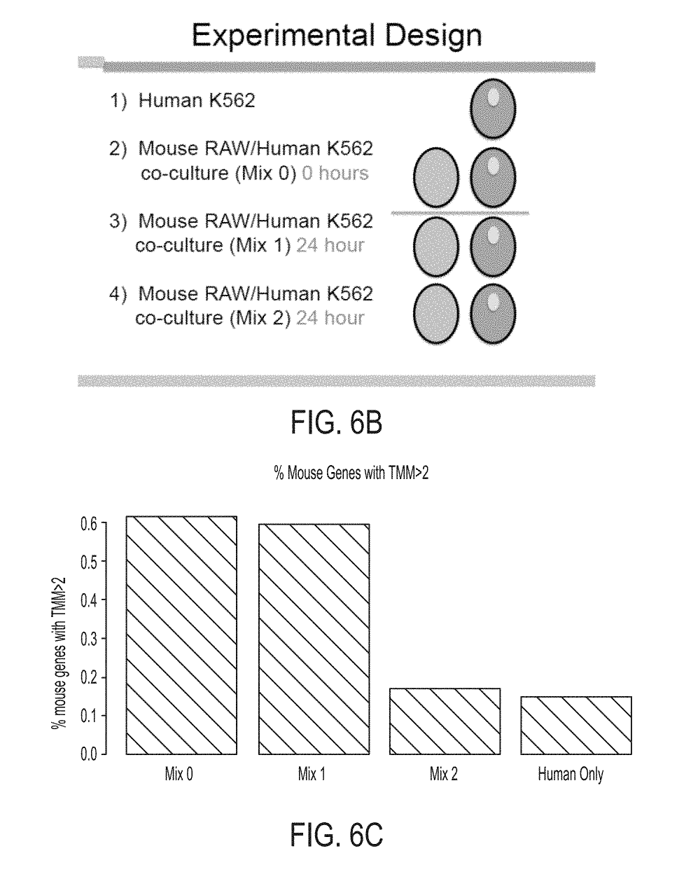

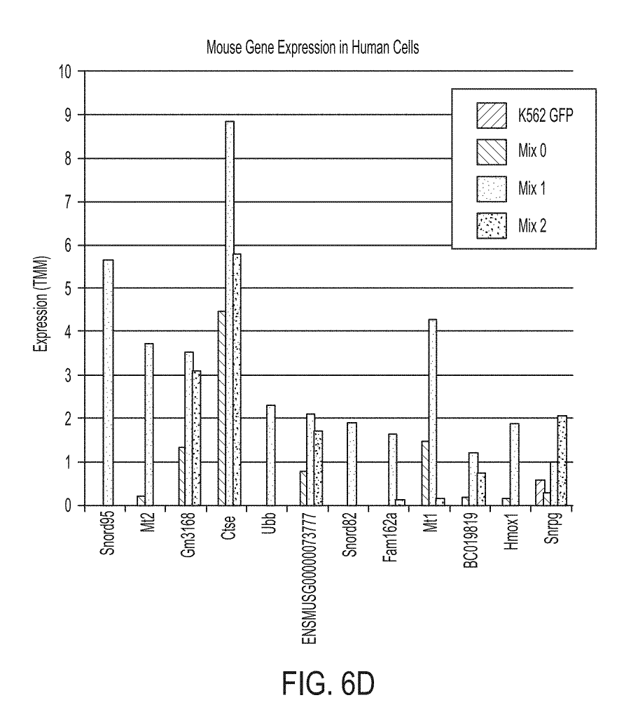

FIGS. 6A-D show principle and results of an experiment to assess possible exosome mediated RNA transfer between co-cultured cell lines.

This example illustrates a way to detect potential RNA transfer using unlabeled RNA. The principle is to co-culture mouse and human cells, separate them back out and use regular RNA-Seq to detect mouse transcripts in human cells. This technique relies on a principle similar to that of Example 7, but without using labeled nucleotides. Using this method, it was possible to detect some RNAs transferred but the strongest signal came from two mouse endogenous retrovirus RNAs (labeled as Gm3168 and Ctse).

Mouse Human RNA Transfer Experiments
Human K562 and Mouse RAW Macrophage cells were both obtained from ATCC.
K562 cells were infected with virus expressing GFP.
K562 cells were FACS sorted to all be GFP positive.
K562 GFP cells were co-cultured with Mouse RAW cells for 24 hours or 0 hours (as a control).
K562 GFP cells were FACS sorted for GFP positive cells to separate from Mouse cells after 24 hour co-culture (2 biological replicates: Mix 1 and Mix 2). The 0 hour co-culture was also sorted, as well as a control of just K562 cells that never interacted with mouse cells.
RNA was extracted using MirVana kit (Lifetech),
200 ng cell RNA to 100 μL with H2O. Add 2 μL Turbo DNase (Lifetech), 2 μL Superasin (Life technologies), and 10 μL DNase buffer to each sample.
Incubation at 37° C. for 30 minutes.
Clean and concentrate using Zymo RNA Clean and Concentrate kit according to instructions and elute in 10 μL H₂O.
PolyA Selection using Dynabeads mRNA Purification kit (Lifetech).
Proceed with RNA-Seq library prep protocol as described in: Perturbation of m6A writers reveals two distinct classes of mRNA methylation at internal and 5′ sites. Schwartz S, Mumbach M R, Jovanovic M, Wang T, Maciag K, Bushkin G G, Mertins P, Ter-Ovanesyan D, Habib N, Cacchiarelli D, Sanjana N E, Freinkman E, Pacold M E, Satija R, Mikkelsen T S, Hacohen N, Zhang F, Carr S A, Lander E S, Regev A. Cell Rep. 2014 Jul. 10; 8(1):284-96. doi: 10.1016/j.celrep.2014.05.048. Epub 2014 Jun. 26.

### Exosome Key Results.

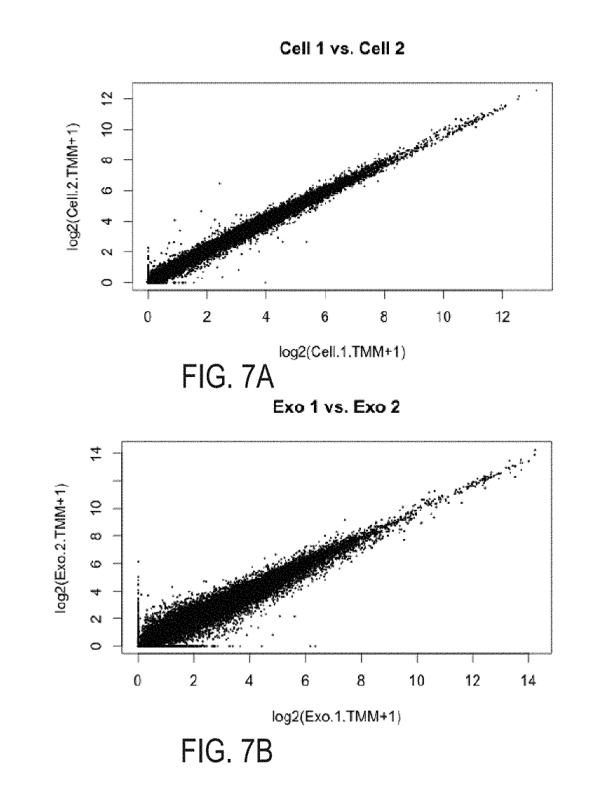

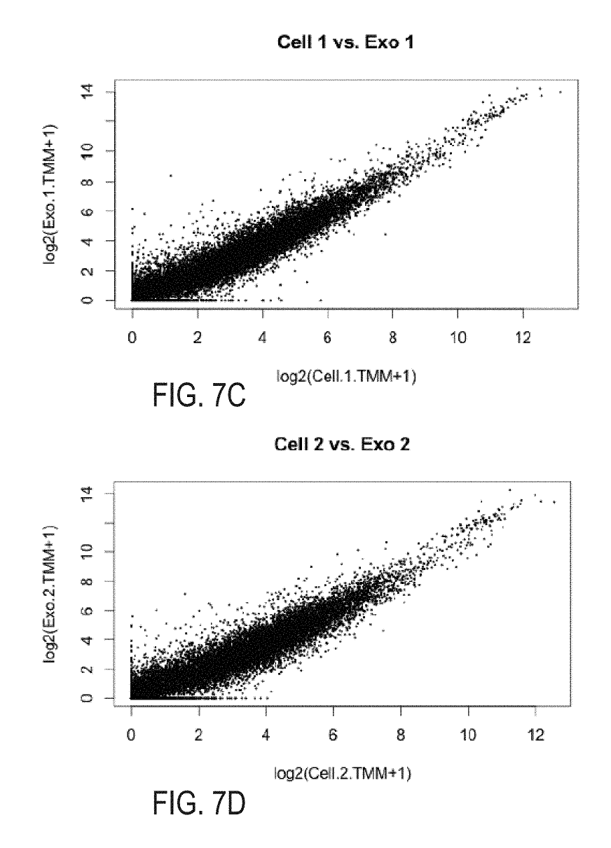

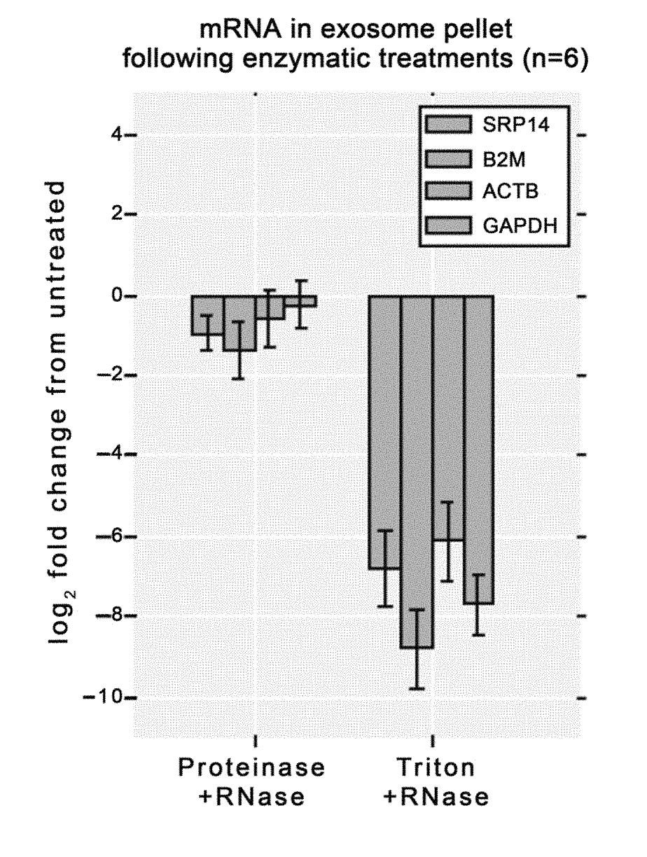

FIGS. 7A-D show poly A selected mRNA from two replicates of K562 cells and their exosomes was compared using RNA-Seq. The bottom two panels show that cell and exosome mRNA is correlated in expression for protein-coding genes.

Applicants have sequenced the mRNA of exosomes from K562 cells and compared the RNA profile of the donor cells to that of the exosomes. Applicants have found that the mRNA profiles of exosomes reflects the trasnscriptome of the donor cells. Thus, using exosomes as a non-invasive read-out of the transcriptome of inaccessible cell types is possible.

FIG. 8 illustrates mRNA in exosome pellet following enzymatic treatments. RNA from untreated exosomes and proteinase/RNase treated exosomes was compared using qRT-PCR for four mRNAs. There was very little or no change, indicating that the RNA is inside. As a control, vesicles with the detergent Triton were lysed and then treated with RNase.

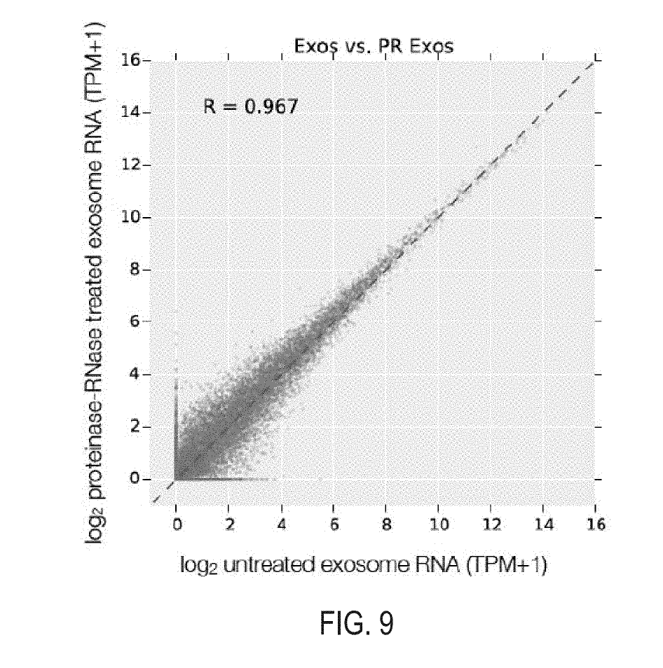

FIG. 9 illustrates Poly A enriched mRNA from untreated exosomes and proteinase/RNAse treated exosomes was compared using RNA-Seq. The mRNA is strongly correlated, indicating that the mRNA isolated via ultracentrifugation in the exosome pellet is inside the vesicles.

Applicants have confirmed that the mRNA in the exosome isolated product is really inside exosomes after developing a protocol to degrade all RNAs not in vesicles by enzymatic treatment with proteinase and then RNAse. Applicants find a very high correlation between the mRNA profiles in the untreated exosome pellet and the proteinase/RNAse treated pellet, indicating the sequenced mRNA is really inside the vesicles. Applicants have confirmed these results through qRT-PCR as well.

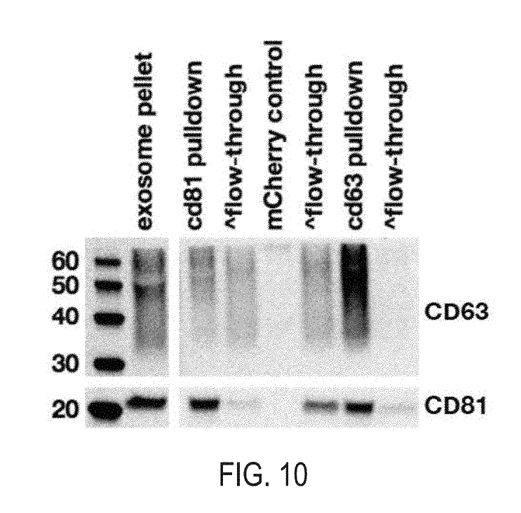

FIG. 10 illustrates targeted pull down exosome subpopulations based on their protein marker using antibody conjugated magnetic beads. CD63 is a glycosylated protein between 30 and 60 kDa. CD81 shows up as a distinct band between 20 and 30 kDa. mCherry is used as a non-specific control. This protocol/technique was developed to isolate specific exosome subpopulations by specific membrane proteins using antibody-conjugated magnetic beads. Further, the technique has been validated in K562 exosomes using the canonical exosome markers CD63 and CD81.

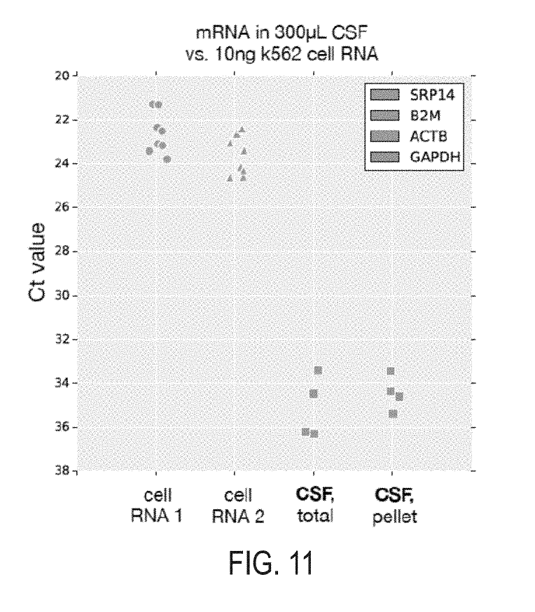

FIG. 11 illustrates exosomes which were isolated from human CSF and mRNA for four genes (detected by qRT-PCR.) Cell RNA is used as a comparison. Two methods of isolating exosomes from CSF were demonstrated: one by running through 0.22 micron filter pelleting at 120,000 g for 2 hours (CSF pellet) and one by extracting RNA directly from CSF after running through 0.22 micron filter without pellet. Similar results were observed by both methods.

### Additional Examples

Mass spectrometry of exosomes from iPS cells and iPS-derived neurons is conducted to find neurons specific membrane proteins found on exosomes. These markers are verified by western blots in iPS and neurons exosomes.

RNA-Seq of exosomes from K562 cells are isolated using CD81 or CD63 antibody-conjugated magnetic beads. The RNA-Seq profiles of exosome subpopulation are compared to the RNA profiles of total exosomes.

RNA-Seq of mRNA from both cells and exosomes from iPS cells and iPS-derived neurons.

RNA-Seq of exosomes from iPS and neuron exosomes isolated using antibody-conjugated magnetic beads to enrich for exosomes expressing the cell type specific proteins. The RNA-Seq profiles of these exosome subpopulations are compared to the RNA profiles of total exosomes from each cell type.

In vitro proof of principle by mixing experiments where Applicants mix cell culture media from iPS cells and neurons and isolate exosomes from the mixed media. Applicants isolate exosomes from the original cell type using antibody-conjugated magnetic beads using the cell type specific markers. Applicants isolate RNA from these exosome subpopulations and perform RNA-Seq to confirm reconstruction of the transcriptome of the original cell type (iPS cells or neurons).

Applicants isolate exosomes from human cerebrospinal fluid (CSF) and perform RNA-Seq.

Applicants enrich for neuron specific exosomes in CSF using antibody-conjugated magnetic beads or a microfluidic device with immobilized antibodies. Applicants then sequence the RNA from these neuron-derived exosomes and to observe enriched expression of neuron-specific genes relative to total CSF exosomes.

The invention is further described by the following numbered paragraphs:

1. A method for the isolation of exosomes from a biological sample, said method comprising:

(a) providing a biological sample comprising exosomes from a cell population,
(b) preparing an exosome-enriched fraction from the biological sample of step (a),
(c) subjecting the exosome-enriched fraction of step (b) to a treatment with a proteinase.

2. A method for the purification of exosomes from a biological sample, said method comprising:

(a) providing a biological sample comprising exosomes from a cell population,
(b) preparing an exosome-enriched fraction from the biological sample of step (a),
(c) subjecting the exosome-enriched fraction of step (b) to a treatment with a proteinase.

3. The method of any one of the preceding numbered paragraphs, wherein the proteinase of step (c) is one or more independently selected from serine proteases, threonine proteases, cysteine proteases, aspartate proteases, glutamic acid proteases and metalloproteases.

4. The method of any one of the preceding numbered paragraphs, wherein step (c) comprises a treatment with a proteinase and subsequent inactivation thereof.

5. The method of the preceding numbered paragraph, wherein proteinase inactivation is performed with one or more protease inhibitor(s).

6. The method of any one of the preceding numbered paragraphs, wherein the proteinase of step (c) is proteinase K.

7. The method of any one of the preceding numbered paragraphs, wherein step (b) comprises one or more centrifugation steps, so as to remove live cells, dead cells and larger cellular debris from the biological sample of step (a).

8. The method of any one of the preceding numbered paragraphs, wherein step (b) comprises one or more filtration steps.

9. The method of the preceding numbered paragraph, wherein the filtration step comprises filtration with a submicron filter.

10. The method of the preceding numbered paragraph, wherein the submicron filter is a 0.22 micron filter.

11. The method of any one of the preceding numbered paragraph, wherein step (b) comprises one or more centrifugation steps, so as to remove live cells, dead cells and larger cellular debris from the biological sample of step (a), followed by a filtration step with a submicron filter.

12. The method of any one of the preceding numbered paragraph, wherein step (b) comprises one or more ultracentrifugation steps.

13. The method of any one of the preceding numbered paragraph, wherein step (b) comprises:

(b-1) filtrating with a submicron filter,

(b-2) performing a first ultracentrifugation step, so as to provide a first exosome-enriched fraction,

(b-3) washing the exosome-enriched fraction of step (b-2), and

(b-4) performing a second ultracentrifugation step of the washed exosome-enriched fraction of step (b-3).

14. The method of any one of numbered paragraphs 12-13, wherein step (c) is performed after the final ultracentrifugation step of step (b).

15. The method of any one of the preceding numbered paragraphs, wherein step (c) comprises a treatment with proteinase K and subsequent inactivation thereof.

16. The method of the preceding numbered paragraph, wherein the inhibitor is diisopropyl fluorophosphate (DFP) or phenyl methane sulphonyl fluoride (PMSF).

17. The method of any one of the preceding numbered paragraphs, further comprising:

(d) subjecting the proteinase K-treated fraction of step (c) to a treatment with an RNase.

18. The method of the preceding numbered paragraph, wherein the RNase is one or more independently selected from RNase A, B, C, 1, and T1.

19. The method of the preceding numbered paragraph, wherein the RNase is RNAse A/T1.

20. The method of any one of numbered paragraphs 17-19, wherein step (d) comprises a treatment with RNase and subsequent inactivation thereof.

21. The method of the preceding numbered paragraph, wherein inactivation of RNase is comprises a treatment with one or more RNase inhibitor(s).

22. The method of the preceding numbered paragraph, wherein the RNase inhibitor is selected from protein-based RNase inhibitors.

23. The method of any one of the preceding numbered paragraphs, wherein the method provides exosomes which are essentially free of extra-exosomal material.

24. The method of any one of the preceding numbered paragraphs, wherein the method provides exosomes which are essentially free of extra-exosomal nucleic acid-protein complexes.

25. The method of any one of the preceding numbered paragraphs, wherein the method provides exosomes which are essentially free of extra-exosomal RNA-protein complexes.

26. The method of any one of the preceding numbered paragraphs, wherein the method further comprises after step (c) or (d) one or more purification steps based on the affinity of a bait molecule for a prey exosome biomarker.

27. The method of any one of the preceding numbered paragraphs, wherein the cell population comprises one or more cell types, 2 or more cell types, preferably 3 or more cell types, 4 or more cell types or 5 or more cell types.

28. The method of any one of the preceding numbered paragraphs, wherein the method isolates or purifies cell type-specific exosomes, or cell-subtype-specific exosomes.

29. The method of any one of the preceding numbered paragraphs, wherein the one or more cell type comprises cells derived from the endoderm, cells derived from the mesoderm, or cells derived from the ectoderm.

30. The method of numbered paragraph 29, wherein cells derived from the endoderm comprise cells of the respiratory system, the intestine, the liver, the gallbladder, the pancreas, the islets of Langerhans, the thyroid or the hindgut.

31. The method of numbered paragraph 29, wherein cells derived from the mesoderm comprise osteochondroprogenitor cells, muscle cells, cells from the digestive systems, renal stem cells, cells from the reproductive system, bloods cells or cells from the circulatory system (such as endothelial cells).

32. The method of numbered paragraph 29, wherein cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

33. The method of numbered paragraph 32, wherein cells from the central nervous system and the peripheral nervous system comprise neurons, Schwann cells, satellite glial cells, oligodendrocytes or astrocytes.

34. The method of numbered paragraph 33, wherein neurons comprise interneurons, pyramidal neurons, gabaergic neurons, dopaminergic neurons, serotoninergic neurons, glutamatergic neurons, motor neurons from the spinal cord, or inhibitory spinal neurons.

35. The method of any one of numbered paragraphs 27 to 29, wherein the one or more cell-type is a cancer cell or a circulating tumor cell (CTC), such as cancer cell or CTC derived from any cell-types or cell subtypes as defined in numbered paragraphs 29 to 34.

36. The method of any one of numbered paragraphs 26 to 35, wherein the prey exosome biomarker comprises a surface biomarker.

37. The method of numbered paragraph 36 wherein the prey exosome biomarker comprises a membrane protein.

38. The method of numbered paragraph 36 or 37 wherein the prey biomarker is selected from the group comprising proteins as per Table D, column G; or proteins as per Table D, column H; or proteins as per Table D, column I; or proteins as per Table D, column J; or proteins as per Table D, column K; or proteins as per Table D, column L; or proteins as per Table D, column M; preferably the prey exosome biomarker is FLRT3 and/or L1CAM.

39. The method of any one of numbered paragraphs 26 to 38, wherein the bait molecule comprises a protein and preferentially an antibody, such as a monoclonal antibody or RNA aptamer.

40. The method of any one of numbered paragraphs 26 to 39, wherein the bait molecule is recognized by an affinity ligand.

41. The method of numbered paragraph 40, wherein the affinity ligand comprises a protein, a peptide, a divalent metal-based complex or an antibody.

42. The method of numbered paragraph of any one of numbered paragraphs 26 to 41, wherein the bait molecule or the affinity ligand is immobilized on a solid substrate.

43. The method of numbered paragraph 42, wherein the solid substrate is selected from a purification column, a microfluidic channel or beads, such as magnetic beads.

44. The method of numbered paragraph any one of numbered paragraph 26 to 43, wherein the purification comprises a microfluidic affinity based purification, a magnetic based purification, a pull-down purification or a fluorescence activated sorting-based purification.

45. The method of any one of the preceding numbered paragraphs, wherein the biological sample comprises a body fluid or is derived from a body fluid, wherein the body fluid was obtained from a mammal.

46. The method of the preceding numbered paragraph, wherein the body fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

47. A method for the isolation of exosomes from a cell population, comprising steps of:

(1) providing isolated exosomes from a biological sample comprising exosomes from said cell population,
(2) performing on the isolated exosomes of step (1) one or more purification steps based on the affinity of a bait molecule for a prey exosome biomarker.

48. A method for the purification of exosomes from a cell population, comprising steps of:

(1) providing purified exosomes from a biological sample comprising exosomes from said cell population,
(2) performing on the purified exosomes of step (1) one or more purification steps based on the affinity of a bait molecule for a prey exosome biomarker.

49. The method of numbered paragraph 47 or 48, wherein step (1) comprises the method for the isolation or the purification of exosomes from a biological sample as defined in numbered paragraphs 1 to 25.

50. The method of any one numbered paragraphs 47 to 49, wherein the cell population comprises one or more cell types, 2 or more cell types, 3 or more cell types, 4 or more cell types or 5 or more cell types.

51. The method of any one numbered paragraphs 47 to 50, wherein the method isolates or purifies cell type-specific exosomes, or cell-subtype-specific exosomes.

52. The method of any one of numbered paragraphs 47 to 51, wherein the one or more cell type comprises from cells derived from the endoderm, cells derived from the mesoderm, and cells derived from the ectoderm.

53. The method of numbered paragraph 52, wherein cells derived from the endoderm comprise cells of the respiratory system, the intestine, the liver, the gallbladder, the pancreas, the islets of Langerhans, the thyroid or the hindgut.

54. The method of numbered paragraph 52, wherein cells derived from the mesoderm comprise osteochondroprogenitor cells, muscle cells, cells from the digestive systems, renal stem cells, cells from the reproductive system, bloods cells or cells from the circulatory system (such as endothelial cells).

55. The method of numbered paragraph 52, wherein cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

56. The method of numbered paragraph 55, wherein cells from the central nervous system and the peripheral nervous system comprises neurons, Schwann cells, satellite glial cells, oligodendrocytes or astrocytes.

57. The method of numbered paragraph 56, wherein neurons comprise interneurons, pyramidal neurons, gabaergic neurons, dopaminergic neurons, serotoninergic neurons, glutamatergic neurons, motor neurons from the spinal cord, or inhibitory spinal neurons.

58. The method of any one of numbered paragraphs 50 to 52, wherein the cell-type is a cancer cell or a circulating tumor cell (CTC), such as a cancer cell or a CTC derived from any cell-types or cell subtypes as defined in numbered paragraphs 52 to 57.

59. The method of any one of numbered paragraphs 47 to 58, wherein the prey exosome biomarker comprises a surface biomarker.

60. The method of numbered paragraph 59, wherein the prey exosome biomarker comprises a membrane protein.

61. The method of any of numbered paragraph 59 to 60, wherein the prey biomarker is selected from the group comprising proteins as per Table D, column G; or proteins as per Table D, column H; or proteins as per Table D, column I; or proteins as per Table D, column J; or proteins as per Table D, column K; or proteins as per Table D, column L; or proteins as per Table D, column M; preferably the prey exosome biomarker is FLRT3 and/or L1CAM.

62. The method of any one of numbered paragraphs 47 to 61, wherein the bait molecule comprises a protein and more preferentially an antibody, such as a monoclonal antibody or RNA aptamer.

63. The method of any one of numbered paragraphs 47 to 62, wherein the bait molecule is recognized by an affinity ligand.

64. The method of numbered paragraph 63, wherein the affinity ligand comprises a protein, a peptide, a divalent metal-based complex or an antibody.

65. The method of any one of numbered paragraphs 47 to 64, wherein the bait molecule or the affinity ligand is immobilized on a solid substrate.

66. The method of numbered paragraph 65 wherein the solid substrate is selected from a purification column, a microfluidic channel or beads, such as magnetic beads.

67. The method of any one of numbered paragraphs 47 to 66, wherein the one or more purification steps comprises a microfluidic affinity based purification, a magnetic based purification, a pull-down purification or a fluorescence activated sorting-based purification.

68. A method for the preparation of exosomal RNA from a biological sample, said method comprising:

(i) providing a biological sample comprising exosomes from a cell population,
(ii) preparing purified exosomes from the biological sample of step (i),
(iii) extracting RNA from the purified exosomes of step (ii).

69. The method of numbered paragraph 68, wherein step (ii) comprises the method of any one of numbered paragraphs 1-46.

70. The method of any one of numbered paragraph 68 or 69, wherein the purified exosomes prepared at step (ii) are exosomes from a single cell type or from a single cell-subtype.

71. A method for the preparation of exosomal RNA of a cell population, comprising steps of

(1) providing purified exosomes from a biological sample comprising exosomes from said cell population,
(2) performing on the purified exosomes of step (1) one or more purification steps based on the affinity of a bait molecule for a prey exosome biomarker, and
(3) extracting RNA from the purified exosomes of step (2).

72. The method of numbered paragraph 71 wherein step (1) comprises the method of any one of numbered paragraphs 1-25.

73. The method of any one of numbered paragraph 71 or 72 wherein step (2) is performed as defined in any one of numbered paragraphs 26 to 46 or as defined in any one of numbered paragraphs 47 to 67.

74. The method of any one of numbered paragraphs 71 to 73, wherein the exosomal RNA is total exosomal RNA.

75. The method of any one of numbered paragraphs 71 to 74, wherein the exosomal RNA comprises exosomal messenger RNA.

76. The method of any one of numbered paragraphs 71 to 75, wherein the exosomal RNA is total exosomal messenger RNA.

77. The method of any one of numbered paragraphs 71 to 76 wherein the exosomal RNA is exosomal RNA from single cell type exosomes or single cell subtype exosomes.

78. Use of a proteinase in the purification of exosomes from a biological sample.

79. Use of a proteinase and of an RNase in the purification of exosomes from a biological sample.

80. Use of a proteinase in the purification of an ultracentrifugated exosome-containing sample.

81. Use of a proteinase and of an RNase in the purification of an ultracentrifugated exosome-containing sample.

82. Use according to any one of numbered paragraphs 78 to 81, wherein the proteinase is proteinase K.

83. Use according to any one of numbered paragraphs 78 to 82, wherein the ultracentrifugated exosome-containing sample is a washed ultracentrifugated exosome-containing sample.

84. Use according to any one of numbered paragraphs 78 to 83, wherein the ultracentrifugated exosome-containing sample is a washed ultracentrifugated exosome-containing sample.

85. The method or use of any one of the preceding numbered paragraphs, wherein the biological sample is a bodily fluid or is derived from a bodily fluid, wherein the bodily fluid was obtained from a mammal.

86. The method or use of the preceding numbered paragraph, wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

87. The method or use of any one of the preceding numbered paragraphs, wherein the cell population is a population of cells of the same cell type.

88. The method or use of any one of the preceding numbered paragraphs, wherein the cell population is a population of cells of different cell types.

89. The method or use of any one of the preceding numbered paragraphs, wherein the cell population comprises one or more cell types, 2 or more cell types, 3 or more cell types, 4 or more cell types, or 5 or more cell types.

90. The method or use of any one of the preceding numbered paragraphs, wherein the biological sample comprises cultured cells.

91. The method or use of any one of the preceding numbered paragraphs, wherein the biological sample comprises cells cultured in vitro.

92. The method or use of any one of the preceding numbered paragraphs, wherein the biological sample comprises cells cultured ex vivo.

93. The method or use of any one of the preceding numbered paragraphs, wherein the biological sample is a sample obtained by liquid biopsy.

94. The method or use of any one of the preceding numbered paragraphs, wherein the biological sample comprises a cell type selected from cells types present in amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit.

95. An exosome preparation obtainable with the method or the use of any one of the preceding numbered paragraphs.

96. A composition comprising exosomes, wherein the composition is essentially free of extra-exosomal material.

97. A composition comprising exosomes, wherein the composition is essentially free of extra-exosomal nucleic acid-protein complexes.

98. A composition comprising exosomes, wherein the composition is essentially free of extra-exosomal RNA-protein complexes.

99. A composition comprising cell type specific exosomes or cell subtype specific exosomes.

100. The composition of numbered paragraph 99, wherein the exosomes are specific for one or more cell types or cell subtypes.

101. The composition of numbered paragraph 100 comprising purified exosomes, wherein said purified exosomes are exosomes from a single cell-type or of a single cell subtype.

102. A method for the determination of cellular RNA content in a cell population, said method comprising:

(a) providing a biological sample comprising exosomes from said cell population,

(b) preparing purified exosomes from the sample of step (a),

(c) extracting RNA from the purified exosomes of step (b), so as to provide exosomal RNA,

(d) analyzing the exosomal RNA extracted at step (c),

(e) estimating, as a function of the result from step (d), the cellular RNA content in the cell population.

103. The method of the preceding numbered paragraph, wherein step (b) comprises the method for the purification of exosomes as disclosed in any of numbered paragraphs 1 to 46.

104. The method of numbered paragraph 102 or 103, wherein step (B) comprises the method for the purification of exosomes from a cell population as disclosed in any of numbered paragraphs 48 to 67.

105. A method for the determination of cellular RNA content of a cell population, said method comprising:

(a) providing a biological sample comprising exosomes from said cell population;

(b) preparing purified exosomes from the sample of step (a);

(d) extracting RNA from the purified exosomes of step (b), so as to provide exosomal RNA;

(d) analyzing the exosomal RNA extracted at step (c);

(e) estimating, as a function of the result from step (d), the cellular RNA content in the cell population

wherein step (b) further comprises performing on the purified exosomes one or more purification steps based on the affinity of a bait molecule for a prey exosome biomarker.

106. The method of numbered paragraph 105, wherein step (b) comprises the method for the isolation or the purification of exosomes from a biological sample as defined in numbered paragraphs 1 to 25.

107. The method of numbered paragraphs 105 or 106, wherein the cell population comprises one or more cell types, 2 or more cell types, 3 or more cell types, 4 or more cell types, 5 or more cell types.

108. The method of any one numbered paragraphs 105 to 107, wherein the method isolates or purifies cell type-specific exosomes, or cell subtype-specific exosomes.

109. The method of any one of numbered paragraphs 105 to 108, wherein the cell type comprises cells derived from the endoderm, cells derived from the mesoderm or cells derived from the ectoderm.

110. The method of numbered paragraph 109, wherein cells derived from the endoderm comprise cells of the respiratory system, the intestine, the liver, the gallbladder, the pancreas, the islets of Langerhans, the thyroid or the hindgut.

111. The method of numbered paragraph 109, wherein cells derived from the mesoderm comprise osteochondroprogenitor cells, muscle cells, cells from the digestive systems, renal stem cells, cells from the reproductive system, bloods cells or cells from the circulatory system (such as endothelial cells).

112. The method of numbered paragraph 109, wherein cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

113. The method of numbered paragraph 112, wherein cells from the central nervous system and the peripheral nervous system comprises neurons, Schwann cells, satellite glial cells, oligodendrocytes or astrocytes.

114. The method of numbered paragraph 113, wherein neurons comprise interneurons, pyramidal neurons, gabaergic neurons, dopaminergic neurons, serotoninergic neurons, glutamatergic neurons, motor neurons from the spinal cord, or inhibitory spinal neurons.

115. The method of any one of numbered paragraphs 107 to 109, wherein the cell-type is a cancer cell or a circulating tumor cell (CTC), such as a cancer cell or a CTC derived from any cell-types or cell subtypes as defined in numbered paragraphs 107 to 114.

116. The method of any one of numbered paragraphs 105 to 115, wherein the prey exosome biomarker comprises a surface biomarker.

117. The method of numbered paragraph 116, wherein the prey exosome biomarker comprises a membrane protein.

118. The method of numbered paragraph 116 or 117, wherein the prey exosome biomarker is selected from the group comprising proteins as per Table D, column G; or proteins as per Table D, column H; or proteins as per Table D, column I; or proteins as per Table D, column J; or proteins as per Table D, column K; or proteins as per Table D, column L; or proteins as per Table D, column M; preferably the prey exosome biomarker is FLRT3 and/or L1CAM.

119. The method of any one of numbered paragraphs 105 to 118, wherein the bait molecule comprises a protein and more preferentially an antibody, such as a monoclonal antibody or RNA aptamer.

120. The method of any one of numbered paragraphs 105 to 119, wherein the bait molecule is recognized by an affinity ligand.

121. The method of numbered paragraph 120, wherein the affinity ligand comprises a protein, a peptide, a divalent metal-based complex or an antibody.

122. The method of numbered paragraph 120 or 121, wherein the bait molecule or the affinity ligand is immobilized on a solid substrate.

123. The method of numbered paragraph 122, wherein the solid substrate is selected from a purification column, a microfluidic channel or beads such as magnetic beads.

124. The method of any one of numbered paragraphs 105 to 123, wherein the purification is comprises a microfluidic affinity based purification, a magnetic based purification, a pull-down purification or a fluorescence activated sorting-based purification.

125. The method of any one of numbered paragraphs 102 to 124, wherein step (e) is performed based on a predicted correlation between exosomal RNA content and cellular RNA content.

126. The method of any one of numbered paragraphs 102 to 125, wherein said determination comprises a qualitative determination.

127. The method of any one of numbered paragraphs 102 to 126, wherein said determination comprises a quantitative determination.

128. The method of any one of numbered paragraphs 102 to 127, wherein said quantitative determination comprises determination of relative abundance of two RNAs.

129. The method of any one of numbered paragraphs 102 to 128, wherein said determination comprises determination of mRNA profiles.

130. The method of any one of numbered paragraphs 102 to 129, wherein said RNA comprises messenger RNA (mRNA).

131. The method of any one of numbered paragraphs 102 to 130, wherein said RNA comprises micro RNA (miRNA).

132. The method of any one of numbered paragraphs 102 to 131, wherein said RNA comprises long non-coding RNA (IncRNA).

133. The method of any one of numbered paragraphs 102 to 132, wherein step (D) comprises a qualitative determination.

134. The method of any one of numbered paragraphs 102 to 133, wherein step (D) comprises a quantitative determination.

135. The method of any one of numbered paragraphs 102 to 134, wherein step (D) comprises RNA sequencing (RNA seq).

136. The method of any one of numbered paragraphs 102 to 135, wherein step (D) comprises array analysis.

137. The method of any one of numbered paragraphs 102 to 136, wherein step (D) comprises reverse transcription polymerase chain reaction (RT-PCR).

138. The method of numbered paragraph 137, wherein step (d) comprises quantitative reverse transcription polymerase chain reaction (qRT-PCR).

139. The method of any one of numbered paragraphs 102 to 138, wherein step (d) comprises analyzing one or more sequence/s of interest.

140. The method of numbered paragraph 139, comprising testing for the presence or absence of said sequence/s of interest.

141. The method of numbered paragraph 140, wherein step (d) comprises analyzing for one or more allelic variants of a sequence of interest.

142. The method according to numbered paragraphs 102 to 141, wherein step (d) comprises testing for presence or absence of said allelic variants.

143. The method of any one of numbered paragraphs 102 to 142, wherein step (d) comprises genome-wide analysis.

144. The method of any one of numbered paragraphs 102 to 143, wherein step (d) comprises transcriptome profiling.

145. The method of any one of numbered paragraphs 102 to 144, wherein the determination is time-lapse.

146. The method of any one of numbered paragraphs 102 to 145, wherein the cell population is a population of cells of the same cell type.

147. The method of any one of numbered paragraphs 102 to 146, wherein the cell population is a population of cells of different cell types.

148. The method of any one of numbered paragraphs 102 to 147, wherein the biological sample comprises cultured cells.

149. The method of any one of numbered paragraphs 102 to 148, wherein the biological sample comprises cells cultured in vitro.

150. The method of any one of numbered paragraphs 102 to 149, wherein the biological sample comprises cells cultured ex vivo.

151. The method of any one of numbered paragraphs 102 to 150, wherein the biological sample is a sample obtained by liquid biopsy.

152. The method of any one of numbered paragraphs 102 to 151, wherein the biological sample comprises a cell type selected from blood, epithelia, muscle and neural cell types.

153. The method of any of numbered paragraphs 102 to 152, wherein the biological sample is obtained from a body fluid selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof.

154. The method of any one of numbered paragraphs 102 to 153, wherein the cell population of step (a) is isolated as a subpart of a larger initial cell population.

155. The method of any one of numbered paragraphs 102 to 154, wherein the cell population of step (a) is obtained from a body fluid and isolated by immuno-magnetic separation.

156. The method of any one of any one of numbered paragraphs 102 to 155, for use in diagnosis.

157. The method of any one of numbered paragraphs 102 to 156, for use in prognosis.

158. The method of any one of numbered paragraphs 102 to 157, for use in identifying markers.

159. The method of any one of numbered paragraphs 102 to 158, for use in a screening process.

160. The method of any one of numbered paragraphs 102 to 159, wherein the method determines the cellular RNA content of a single cell type or of a single cell subtype.

161. A method for the diagnostic or prognostic of a disorder of interest in a subject, comprising:

(I) selecting a marker, wherein said marker is associated with said disorder and wherein said marker may be determined in a cell type that is found in the subject to be in contact with a body fluid,
(II) providing a biological sample from said body fluid from said subject,
(III) estimating the cellular RNA content of said marker in the biological sample of step (II) by performing the method of any one of numbered paragraphs 102 to 155.

162. The method of numbered paragraph 161, wherein the cellular RNA content is the cellular content of a single cell type or of a single cell subtype.

163. The method of numbered paragraph 161 or 162, further comprising (IV) determining, from the results of step (III), the status of the marker selected at step (I).

164. The method of any one of numbered paragraphs 160 to 163, wherein the marker is selected from expression of a given open reading frame (ORF), overexpression of a given open reading frame (ORF), repression of a given open reading frame (ORF), over-repression of a given open reading frame (ORF), expression of a given allelic variant, relative level of expression of a given open reading frame (ORF), presence of a mutation in a given open reading frame (ORF),

165. The method of any one of numbered paragraphs 161 to 164, wherein said disorder is a blood disorder and said marker is a marker that may be determined in one or more cell type/s that is/are found in the subject to be in contact with blood.

166. The method of any one of numbered paragraphs 161 to 165, wherein said disorder is a brain or spine disorder and said marker is a marker that may be determined in one or more cell type/s that is/are found in the subject to be in contact with cerebrospinal fluid.

167. The method of any one of numbered paragraphs 161 to 166, wherein said disorder is a heart disorder and said marker is a marker that may be determined in one or more cell type/s that is/are found in the subject to be in contact with blood or pericardial fluid.

168. The method of any one of numbered paragraphs 161 to 167, wherein said disorder is a prostate or bladder disorder and said marker is a marker that may be determined in one or more cell type/s that is/are found in the subject to be in contact with urine.

169. The method of any one of numbered paragraphs 161 to 168, wherein said disorder is an eye disorder and said marker is a marker that may be determined in one or more cell type/s that is/are found in the subject to be in contact with tears.

170. The method of any one of numbered paragraphs 161 to 169, wherein said disorder is a lung disorder and said marker is a marker that may be determined in one or more cell type/s that is/are found in the subject to be in contact with pleural fluid.

171. Composition comprising exosomes, wherein the composition is essentially free of extra-exosomal material, for use in diagnostics.

172. Composition comprising exosomes, wherein the composition is essentially free of extra-exosomal nucleic acid-protein complexes.

173. Composition comprising exosomes, wherein the composition is essentially free of extra-exosomal RNA-protein complexes.

174. A method for the treatment or prophylaxis of a disorder in a patient, said method comprising exosome-mediated delivery of a therapeutic RNA to a cell.

175. The method of numbered paragraph 174, wherein said exosome-mediated delivery occurs from one donor cell to a recipient cell, and wherein the therapeutic RNA results from transcription in the donor cell.

176. The method of numbered paragraph 175, wherein transcription in the donor cell is inducible.

177. The method of any one of numbered paragraphs 174 to 176, wherein the delivery is performed ex vivo.

178. The method of any one of numbered paragraphs 174 to 176, wherein the delivery is performed in vivo.

179. Exosome for use in delivering a therapeutic RNA to a cell.

180. Exosome of numbered paragraph 179, wherein the exosome is produced according to the method or the use as defined in any one of numbered paragraphs 1 to 70 and 78 to 94.

181. Exosome of numbered paragraph 179, wherein the exosome is in a preparation obtainable according to numbered paragraph 95.

182. Exosome of numbered paragraph 179, wherein the exosome is produced in vitro

183. Exosome of numbered paragraph 179, wherein the exosome is produced in vivo.

184. Therapeutic RNA for use in exosome-mediated delivery to a cell.

185. Therapeutic RNA of numbered paragraph 184, wherein the exosome is produced in vitro

186. Therapeutic RNA of numbered paragraph 184, wherein the exosome is produced in vivo.

187. Therapeutic RNA of numbered paragraph 184, wherein the exosome is produced according to the method or the use as defined in any one of numbered paragraphs 1 to 70 and 79 to 84.

188. Therapeutic RNA of numbered paragraph 184, wherein the exosome is in a preparation obtainable according to numbered paragraph 95.

189. Pharmaceutical composition comprising an exosome, wherein said exosome comprises a therapeutic RNA for delivery into a cell.

190. The pharmaceutical composition of numbered paragraph 189, wherein the delivery is performed ex vivo.

191. The pharmaceutical composition of numbered paragraph 189, wherein the delivery is performed in vivo.

192. Pharmaceutical composition comprising a cell, wherein the cell is capable of producing exosomes comprising a therapeutic RNA.

193. Pharmaceutical composition of any one of numbered paragraphs any one of numbered paragraphs 189 to 192, in a form suitable for injection.

194. Use of a therapeutic RNA in the manufacture of a medicament for the treatment or prophylaxis of a disorder in a patient, wherein the RNA is delivered to a cell in an exosome-packaged form.

195. Use of an exosome in the manufacture of a medicament for the treatment or prophylaxis of a disorder in a patient, wherein the exosome comprises a therapeutic RNA or delivery into a cell.

196. The method, composition or use of any one of numbered paragraphs 174 to 195, wherein the therapeutic RNA is translated in the recipient cell.

197. The method, composition or use of any one of numbered paragraphs 174 to 195, wherein the therapeutic RNA is a small interfering RNA (siRNA).

198. The method, composition or use of any one of numbered paragraphs 174 to 195, wherein the therapeutic RNA is a short hairpin RNA (shRNA).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of purifying exosomes from a cell population, said method comprising:
(a) preparing an exosome-enriched fraction from a biological sample comprising the exosomes, and
(b) subjecting the exosome-enriched fraction of step (b) to a treatment with a proteinase.

2. The method of claim 1, wherein the proteinase of step (b) comprises one or more proteinase selected from serine proteases, threonine proteases, cysteine proteases, aspartate proteases, glutamic acid proteases and metalloproteases.

3. The method of claim 1, wherein the proteinase of step (b) comprises proteinase K.

4. The method of claim 1, wherein step (b) comprises treatment with a proteinase and subsequent inactivation thereof.

5. The method of claim 4, wherein proteinase inactivation is performed with one or more protease inhibitor(s).

6. The method of claim 4, wherein proteinase inactivation comprises treatment with diisopropyl fluorophosphate (DFP) or phenyl methane sulphonyl fluoride (PMSF).

7. The method of claim 1, wherein step (a) comprises one or more centrifugation steps, so as to remove live cells, dead cells and larger cellular debris from the biological sample.

8. The method of claim 1, wherein step (a) comprises one or more filtration steps.

9. The method of claim 1, wherein step (a) comprises filtration with a submicron filter.

10. The method of claim 9, wherein the submicron filter is a 0.22 micron filter.

11. The method claim 1, wherein step (a) comprises:
(a-1) filtrating with a submicron filter,
(a-2) performing a first ultracentrifugation step, so as to provide a first exosome-enriched fraction,
(a-3) washing the exosome-enriched fraction of step (b-2), and
(a-4) performing a second ultracentrifugation step of the washed exosome-enriched fraction of step (a-3).

12. The method of claim 11, wherein step (b) is performed after the final ultracentrifugation step of step (a).

13. The method of claim 1, further comprising:
(c) subjecting the protease-treated fraction of step (b) to a treatment with an RNase.

14. The method of claim 13, wherein the RNase comprises one or more of RNase A, B, C, 1, and T1.

15. The method of claim 13, wherein the RNase comprises RNAse A.

16. The method of any one of claims 1 or 13, wherein the method further comprises affinity purification after step (b) or (c).

17. The method of claim 1, wherein the cell population comprises one or more cell types, 2 or more cell types, 3 or more cell types, 4 or more cell types or 5 or more cell types.

18. The method of claim 1, wherein the method purifies cell type-specific exosomes, or cell-subtype-specific exosomes.

19. The method of claim 18, wherein the cell type comprises cells derived from the endoderm, cells derived from the mesoderm, or cells derived from the ectoderm.

20. The method of claim 19, wherein cells derived from the endoderm comprise cells of the respiratory system, the intestine, the liver, the gallbladder, the pancreas, the islets of Langerhans, the thyroid or the hindgut.

21. The method of claim 19, wherein cells derived from the mesoderm comprise osteochondroprogenitor cells, muscle cells, cells from the digestive systems, renal stem cells, cells from the reproductive system, bloods cells or cells from the circulatory system (such as endothelial cells).

22. The method of claim 19, wherein cells derived from the ectoderm, comprise epithelial cells, cells of the anterior pituitary, cells of the peripheral nervous system, cells of the neuroendocrine system, cell of the teethes, cell of the eyes, cells of the central nervous system, cells of the ependymal or cells of the pineal gland.

23. The method of claim 22, wherein cells from the central nervous system and the peripheral nervous system comprise neurons, Schwann cells, satellite glial cells, oligodendrocytes or astrocytes.

24. The method of claim 23, wherein neurons comprise interneurons, pyramidal neurons, gabaergic neurons, dopaminergic neurons, serotoninergic neurons, glutamatergic neurons, motor neurons from the spinal cord, or inhibitory spinal neurons.

25. The method of claim 17, wherein the one or more cell-type comprises a cancer cell or a circulating tumor cell (CTC).

26. The method of claim 16, wherein the affinity purification comprises a biomarker from Table D, column G, Table D, column H, Table D, column I, Table D, column J, Table D, column K, Table D, column L, or Table D, column M.

27. The method of claim 26, wherein the biomarker comprises FLRT3 and/or L1CAM.

28. The method of claim 1, wherein the biological sample comprises amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit or mixtures of one or more thereof.

29. A method of determining cellular RNA content in a cell population, said method comprising:
(a) preparing purified exosomes from said cell population according to claim 1,
(b) extracting RNA from the purified exosomes of step (a) to provide exosomal RNA,
(c) analyzing the exosomal RNA of step (b),
(d) estimating the cellular RNA content in the cell population as a function of the result from step (c).

30. The method of claim 29, wherein the proteinase of claim 1 comprises one or more proteinase selected from serine proteases, threonine proteases, cysteine proteases, aspartate proteases, glutamic acid proteases and metalloproteases.

31. The method of claim 30, wherein the proteinase of claim 1 comprises proteinase K.

32. The method of claim 29, wherein the proteinase of claim 1 is inactivated before extracting RNA from the purified exosomes.

33. The method of claim 32, wherein proteinase inactivation is performed with one or more protease inhibitor(s).

34. The method of claim 33, wherein the proteinase comprises proteinase K and proteinase inactivation comprises treatment with diisopropyl fluorophosphate (DFP) or phenyl methane sulphonyl fluoride (PMSF).

35. The method of claim 29, wherein preparing purified exosomes from the cell population further comprises affinity purification of the exosomes.

36. A method of diagnosing or prognosing a disease or disorder in a subject, comprising:
(a) selecting a biomarker in a cell population associated with said disease or disorder of the subject,
(b) preparing purified exosomes from said cell population according to the method of claim 16,
(c) extracting RNA from the purified exosomes of step (b) to provide exosomal RNA,
(d) analyzing the exosomal RNA of step (c),
(e) estimating the cellular RNA content in the cell population as a function of the result from step (d), and
(f) determining the status of the disease or disorder in the subject from the cellular RNA content in the cell population.
